(19) United States
(12) Patent Application Publication
(10) Pub. No.: US 2012/0065254 A1
(43) Pub. Date: Mar. 15, 2012

(54) C5A binding nucleic acids and the use thereof

(75) Inventors: Florian Jarosch, Berlin (DE); Werner Purschke, Berlin (DE); Dirk Eulberg, Berlin (DE); Christian Maasch, Berlin (DE); Klaus Buchner, Berlin (DE); Sven Klussmann, Berlin (DE)

(73) Assignee: NOXXON PHARMA AG, Berlin (DE)

(21) Appl. No.: 13/259,007

(22) PCT Filed: Mar. 23, 2010
(86) PCT No.: PCT/EP2010/001813

§ 371 Date: Nov. 12, 2011

(30) Foreign Application Priority Data
Mar. 23, 2009 (EP) 09004122.9

(51) Int. Cl. A61K 31/7088; C07K 2/00; C07K 14/00; C07H 21/02

(52) U.S. Cl. 514/44 R; 536/23.1; 530/322; 530/358

(57) ABSTRACT

The present invention is related to a nucleic acid molecule, capable of binding to C5a, whereby the nucleic acid molecule is for use in a method for the treatment and/or prevention of tumors, the treatment and/or prevention of a respiratory disease, the treatment and/or prevention of pain and/or the treatment and/or prevention of a neurodegenerative disorder.

The present invention is related to nucleic acids binding to C5a and/or C5, and the use thereof for the manufacture of a medicament and a diagnostic agent, respectively.

The primary structure of the anaphylatoxin C5a (complement factor 5a; SwissProt entry P01031) was determined in 1978 (Fernandez and Hugli, 1978). It consists of 74 amino acids accounting for a molecular weight of 8,200 Da while the carbohydrate portion accounts for approximately 3,000 Da. The carbohydrate portion of C5a exists as a single complex oligosaccharide unit attached to an asparagine at position 64. The three disulfide bonds confer a stable, rigid structure to the molecule.

The tertiary structure of C5a was determined by NMR analysis. The protein consists of four helices juxtaposed in an approximately antiparallel topology connected by peptide loops located at the surface of the molecule (Zuiderweg et al., 1989).

Although the three-dimensional structure of C5a forms from different mammalian species has generally been maintained, the amino acid sequence has not particularly well been conserved during evolution. Sequence alignment results demonstrate 64% overall sequence identity with mouse C5a. Human C5a shares the following percentages of identical amino acids with C5a from:

| | |
|---|---|
| Macaca mulatta (rhesus monkey) | 85% |
| Macaca fascicularis (cynomolgus monkey) | 85% |
| Bos taurus (bovine) | 69% |
| Sus scrofa (pig) | 68% |
| Mus musculus (mouse) | 64% |
| Rattus norvegicus (rat) | 61% |

The more distantly related human proteins C3a and C4a share only 35 and 40% identity with C5a, respectively.

The complement system was discovered at the beginning of the last century as a heat sensitive serum fraction that “complemented” the antisera mediated lysis of cells and bacteria. Being a humoral component of the natural unspecific (innate) immune response, it plays an essential role in host defence against infectious agents and in the inflammatory process. Complement can be activated via three distinct pathways (i) after an antibody attaches itself to a cell surface or bacteria (referred as classical pathway), (ii) directly by bacterial or viral glycolipids (referred as alternative pathway), or (iii) by carbohydrates on bacteria (referred as lectin pathway). All these activation pathways converge at the point of activation of the complement component C5, where the common terminal pathway starts, culminating in assembly of the membrane attack complex (abbr. MAC). The complement system consists of more than 20 soluble proteins that function either as proteolytic enzymes or as binding proteins and making up about 10% of the total globulins in vertebrate serum. In addition, the complement system includes multiple distinct cell-surface receptors that exhibit specificity for proteolytic fragments of complement proteins and that are expressed by inflammatory cells and cells regulating the adaptive immune response. There are several regulatory proteins that inhibit complement activation and thus protect host cells from accidental complement attack. The complement system can become activated independently or together with the adaptive immune response.

The functions of complement include the process of opsonization (i.e. making bacteria more susceptible to phagocytosis), lysis of bacteria and foreign cells by inserting a pore into their membrane (referred as membrane attack complex), generation of chemotactically active substances, increase of vascular permeability, evocation of smooth muscle contraction, and promotion of mast cell degranulation. Similarly to the coagulation cascade, the process of complement activation is organized in sequential enzymatic steps also known as an enzymatic cascade (Sim and Laich, 2000). The detailed sequence of these interactions is outlined in the following:

Classical Pathway. This antibody-dependent activation pathway complements the specific antibody response. It is as elaborately controlled as the alternative pathway, but lacks the spontaneous initiation ability; i.e. the antibody-independent recognition function, and the feedback amplification mechanism. Among the activators of the classical pathway are antigen-antibody complexes, β-amyloid, DNA, polyinosinic acid, polyanion-polycation complexes like heparin/protamine, some enveloped viruses, monosodium urate crystals, lipid A of bacterial cell walls, plicatic acid, ant venom polysaccharide, subcellular membranes (such as mitochondria), as well as cell- and plasma-derived enzymes such as plasmin, kallikrein, activated Hageman factor, elastase or cathepsins. The antibody-induced classical pathway starts with C1, which binds to the Fc-fragment of an antibody (IgM>IgG3>IgG1>>IgG2) ligated to a cell surface antigen. C1 is a recognition complex composed of 22 polypeptide chains in 3 subunits; C1q, C1r, C1s. C1q is the actual recognition portion, a glycoprotein containing a collagen-like domain (exhibiting hydroxyproline and hydroxylysine residues) that looks like a bunch of tulips. Upon binding via C1q, C1r is activated to become a protease that cleaves C1s to a form that activates (by cleavage) both C2 and C4 to C2a/b and C4a/b. C2a and C4b combine to produce C4b2a, the C3 convertase (C3 activating enzyme). C4a has only weak anaphylatoxin activity but is not chemotactic. C3 is central to all three activation pathways. In the classical pathway, C4b2a convertase cleaves C3 into C3a/b. C3a is an anaphylatoxin. C3b combines with C4b2a to form C4b2a3b complex (C5 convertase). C3b can also bind directly to cells making them susceptible to phagocytosis (opsonization).

Alternative pathway. This pathway does not require antibodies for activation and is of major importance in host defence against bacterial and viral infection because—unlike the classical pathway—it is directly activated by surface structures of invading microorganisms such as bacterial/viral glycolipids or endotoxins. Other activators are inulins, rabbit erythrocytes, desialylated human erythrocytes, cobra venom factor, or phosphorothioate oligonucleotides. The six proteins C3, Factors B, D, H, I, and properdin together perform the functions of initiation, recognition and activation of the pathway which results in the formation of activator-bound C3/C5 convertase. The cascade begins with C3. A small amount of C3b is always found in circulation as a result of spontaneous cleavage of C3 (“C3-tickover”), but the concentrations are generally kept very low by subsequent degradation. However, when C3b binds covalently to sugars on a cell surface, it can serve as a nucleus for alternative pathway activation. Then Factor B binds to C3b. In the presence of Factor D, bound Factor B is cleaved to Ba and Bb; Bb contains the active site for a C3 convertase. Next, properdin binds to C3bBb to stabilize the C3bBb convertase on the cell surface leading to cleavage of further C3 molecules. Finally, the alternative C5 convertase C3bBb3b forms which cleaves C5 to C5a/b. Once present, C5b initiates assembly of the membrane attack complex as described above. Generally, only Gram-negative cells can be directly lysed by antibody plus complement; Gram-positive cells are mostly resistant. However, phagocytosis is greatly enhanced by opsonization with C3b (phagocytes have C3b receptors on their surface) and antibody is not always required. In addition, complement can neutralize virus particles either by direct lysis or by preventing viral penetration of host cells.

(3) Lectin pathway. The most recently discovered lectin or mannan-binding lectin (abbr. MBL) pathway depends on innate recognition of foreign substances (i.e., bacterial surfaces). This pathway has structural and functional similarities to the classical pathway. Activation of the lectin pathway is initiated by the acute phase protein MBL, which recognizes mannose on bacteria, IgA and probably structures exposed by damaged endothelium. MBL is homologous to C1q and triggers the MBL associated serine proteases (abbr. MASPs), of which the three forms MASP1, MASP2 and MASP3 have been described. Further lectin pathway activation is virtually identical to classical pathway activation forming the same C3 and C5 convertases. In addition there is some evidence that MASPs under some conditions may activate C3 directly.

(4) Terminal pathway. All three activation pathways converge in the formation of C5 convertase (C4b2a3b in the classical and lectin pathway, C3bBb3b in the alternative pathway), which cleaves C5 to C5a/b. C5a has potent anaphylatoxin activity and is chemotactic. The other C5 fragment C5b functions with its hydrophobic binding site as an anchor on the target cell surface to which the lytic membrane attack complex (MAC or terminal complement complex, abbr. TCC) forms. The MAC is assembled from five precursor proteins: C5b, C6, C7, C8, and C9. The final event is the formation of C9 oligomers, which insert themselves as transmembrane channels into the plasma membrane leading to osmotic lysis of the cell. MAC assembly is controlled by the soluble plasma factors S protein (also so known as vitronectin) and SP-40,40 (also so known as clusterin), and by CD59 and HRF (homologous restriction factor) on host cell membranes. Many kinds of cells are sensitive to complement mediated lysis: erythrocytes, platelets, bacteria, viruses possessing a lipoprotein envelope, and lymphocytes.

The complement system is a potent mechanism for initiating and amplifying inflammation. This is mediated through fragments of the complement components. Anaphylatoxins are the best defined fragments and are proteolytic fragments of the serine proteases of the complement system: C3a, C4a and C5a. Anaphylatoxins are not only produced in the course of complement activation, but also from activation of other enzyme systems which may directly cleave C3, C4 and C5. Such enzymes include plasmin, kallikrein, tissue and leukocyte lysosomal enzymes, and bacterial proteases. The anaphylatoxins have powerful effects on blood vessel walls, causing contraction of smooth muscle (e.g. ileal, bronchial, uterine and vascular muscle) and an increase in vascular permeability. These effects show specific tachyphylaxis (i.e. repeated stimulation induces diminishing responses) and can be blocked by antihistamines; they are probably mediated indirectly via release of histamine from mast cells and basophils. C5a is the 74-amino acid N-terminal cleavage product of the C5 plasmaprotein α chain. It is bound by the receptor C5aR (also known as C5R1 or CD88) with high affinity, a molecule present on many different cell types: most prominently on neutrophils, macrophages, smooth muscle cells, and endothelial cells. C5a is by far the most powerful anaphylatoxin, approximately 100 times more effective than C3a, and 1000 times more effective than C4a. This activity decreases in the order C5a>histamine>acetylcholine>C3a>>C4a.

C5a is extremely potent at stimulating neutrophil chemotaxis, adherence, respiratory burst generation and degranulation. C5a also stimulates neutrophils and endothelial cells to present more adhesion molecules; the intravenous injection of C5a, for example, quickly leads to neutropenia in animal experiments by triggering adherence of neutrophils to the blood vessel walls. Ligation of the neutrophil C5a receptor is followed by mobilization of membrane arachidonic acid which is metabolized to prostaglandins and leukotrienes including LTB4, another potent chemoattractant for neutrophils and monocytes. Following ligation of monocyte C5a receptors, IL-1 is released. Thus, the local release of C5a at sites of inflammation results in powerful pro-inflammatory stimuli. In fact, the release of C5a is connected directly or indirectly with many acute or chronic conditions, such as immune complex associated diseases in general (Heller et al., 1999); asthma (Kohl, 2001); septic shock (Huber-Lang et al., 2001); systemic inflammtory response syndrome (abbr. SIRS); multiorgan failure (abbr. MOF); acute respiratory distress syndrome (abbr. ARDS); inflammatory bowel syndrome (abbr. IBD) (Woodruff et al., 2003); infections; severe burns (Piccolo et al., 1999); reperfusion injury of organs such as heart, spleen, bladder, pancreas, stomach, lung, liver, kidney, limbs, brain, sceletal muscle or intestine (Riley et al., 2000); psoriasis (Bergh et al., 1993); myocarditis; multiple sclerosis (Muller-Ladner et al., 1996); and rheumatoid arthritis (abbr. RA) (Woodruff et al., 2002).

Numerous overviews over the relation between the complement system and diseases are published (Kirschfink, 1997; Kohl, 2001; Makrides, 1998; Walport, 2001a; Walport, 2001b).

Cell injury by complement occurs as a consequence of activation of either the classical or the alternative pathway on the surface of a cell. The MAC constitutes a supramolecular organisation that is composed of approximately twenty protein molecules and representing a molecular weight of approx. 1.7 million Da. The fully assembled MAC contains one molecule each of C5b, C6, C7, and C8 and several molecules of C9. All these MAC components are glycoproteins. When C5 is cleaved by C5 convertase and C5b is produced, self-assembly of the MAC begins. C5b and C6 form a stable and soluble bimolecular complex which binds to C7 and induces it to express a metastable site through which the nascent trimolecular complex (C5b-7) can insert itself into membranes, when it occurs on or in close proximity to a target lipid bilayer. Insertion is mediated by hydrophobic regions on the C5b-7 complex that appear following C7 binding to C5b-6. Membrane-bound C5b-7 commits MAC assembly to a membrane site and forms the receptor for C8. The binding of one C8 molecule to each C5b-7 complex gives rise to small trans-membrane channels of less than 1 nm functional diameter that may perturb target bacterial and erythrocyte membranes. Each membrane-bound C5b-8 complex acts as a receptor for multiple C9 molecules and appears to facilitate insertion of C9 into the hydrocarbon core of the cell membrane. Binding of one molecule of C9 initiates a process of C9 oligomerisation at the membrane attack site. After at least 12 molecules are incorporated into the complex, a discrete channel structure is formed. Therefore the end product consists of the tetramolecular C5b-8 complex (with a molecular weight of approximately 550 kDa) and tubular poly-C9 (with a molecular weight of approximately 1,100 kDa). This form of the MAC, once inserted into the cell membranes, creates complete transmembrane channels leading to osmotic lysis of the cell. The transmembrane channels formed vary in size depending on the number of C9 molecules incorporated into the channel structure. Whereas the presence of poly-C9 is not absolutely essential for the lysis of red blood cells or of nucleated cells, it may be necessary for the killing of bacteria.

The complement system is primarily beneficial in the body's defense against invading microorganisms. The early components of the complement cascade are important for opsonization, of infectious agents followed by their elimination from the body. In addition, they serve several normal functions of the immune system like controlling formation and clearance of immune complexes or cleaning up debris, dead tissues and foreign substances. All three activation pathways which recognize different molecular patterns that (in the healthy body) define an extensive array of non-self structures help controlling invaders. The terminal complement pathway—which culminates in the assembly of the MAC—represents a further line of defense by lysing bacteria and foreign cells.

The importance of a functional complement system becomes clear when the effects of complement deficiencies are considered. For example, individuals that are missing one of the alternative pathway proteins or late components (C3-C9) tend to get severe infections with pyogenic organisms, particularly Neisseria species. Deficiencies in the classical pathway components (such as C1, C2, C4) are also associated with increased, though not as strongly elevated, risk of infection. Complement components like C1 and MBL do also have the ability to neutralize viruses by interfering with the viral interaction with the host cell membrane, thus preventing entrance into the cell.

Of note, although cleavage of C5 leads to C5a as well as the MAC, the clinical features of C5 deficiency do not differ markedly from those of other terminal component deficiencies (e.g. C6, C7, C8, C9) suggesting that the absence of C5a does not contribute significantly to the clinical picture in C5-deficient patients. Therefore, the selective antagonisation of C5a promises to be the optimal leverage, so that the normal up- and downstream disease-preventing functions of complement remain intact. Thus, only the deleterious overproduction of the proinflammatory anaphylatoxin is blocked.

The fact that C5aR-deficient mice—although they are more susceptible for infections with Pseudomonas aeruginosa—appear otherwise normal, suggests that the blockade of C5a function does not have deleterious effects.

Several compounds targeting C5a or C5 or the respective receptor are known and were successfully tested in vivo models. Some of them have been further tested in clinical trials. The C5-specific humanized antibody, eculizumab is approved for paroxysmal nocturnal hemoglobinuria and has shown efficacy in treating atypical haemolytic uraemic syndrome (aHUS), acute antibody-mediated kidney allograft rejection and cold agglutinin disease. It prevents cleavage of C5 and inhibits the action of both C5a and C5b. Besides similar research-stage C5 antibodies and antibody fragments, the C5a antibody TNX-558 is of special interest, since it only disrupts the C5a:C5aR(CD88) interaction and leaves C5 cleavage and C5b-dependent MAC-formation unaffected. An antibody to the C5a receptor, neutrazumab, is under development for rheumatoid arthritis and stroke (Ricklin & Lambris 2007; Wagner & Frank 2010).

A PEGylated anti-C5 aptamer (ARC-1905) is in preclinical development for AMD.

No development has been reported for the small molecule/peptidomimetic C5a receptor antagonists JPE-1375, JSM-7717 recently (Ricklin & Lambris 2007). Another inhibitor of the C5a receptor CD88, the cyclic hexapeptide PMX53, has been efficacious in inflammatory animal models, but has not met endpoints in placebo-controlled double-blind clinical studies in patients with rheumatoid arthritis. The clinical development for AMD has also been discontinued (Wagner & Frank 2010). A research-stage variant of PMX53, PMX205, has been published to be ative in a murine model of Alzheimer's dementia (Fonseca et al. 2009). A further clinical stage compound is the C5a receptor (C5aR) antagonist, CCX-168. A Phase I trial has been initiated for inflammatory and autoimmune diseases in January 2010.

Beside the effects of C5a as described supra, new data let assume that the generation of C5a in a tumor microenviroment enhance tumor growth by the suppression the antitumor CD8+ T-cell-mediated response, whereby said suppression seems to be associated with the recruitement of myeloid-dereived supressor cells into tumors and augmention of their T-cell-directed supressive abilities. Markiewski et al. showed that a blockade of the C5a receptors by a pepdidic C5a receptor antagonist led to a retarded tumor growth in a mouse model (Markiewski et al., 2008).

Most of peptidic compounds are prone to degradation and modification by peptidases and additionally show a fast clearance rate from the body, preferably the human body. Thus, these peptidic compounds cannot be considered as drag-like molecules, a prerequisite for the development of drugs in general to be marketed.

The problem underlying the present invention is to provide a drug-like molecule which specifically interacts with C5a. More specifically, the problem underlying the present invention is to provide for a nucleic acid based drug-like molecule which specifically interacts with C5a.

A further problem underlying the present invention is to provide a drug-like molecule for the manufacture of a medicament for the treatment of a human or non-human diseases, whereby the disease is characterized by C5a being either directly or indirectly involved in the pathogenetic mechanism of such disease.

A still further problem underlying the present invention is to provide a drug-like molecule for the manufacture of a diagnostic agent for the treatment of a disease, whereby the disease is characterized by C5a being either directly or indirectly involved in the pathogenetic mechanism of such disease.

These and other problems underlying the present invention are solved by the subject matter of the attached independent claims. Preferred embodiments may be taken from the dependent claims.

In a first aspect which is also a first embodiment of the first aspect, the problem underlying the instant invention is solved by a nucleic acid molecule, capable of binding to C5a, whereby the nucleic acid molecule is for use in a method for the treatment and/or prevention of tumors, the treatment and/or prevention of a respiratory disease, the treatment and/or prevention of pain and/or the treatment and/or prevention of a neurodegenerative disorder.

In a second embodiment of the first aspect which is also an embodiment of the first embodiment of the first aspect, the nucleic acid molecule is for use in a method for the treatment and/or prevention of tumors, wherein the tumors are selected from the group of tumors of the endocrine system, tumors of the eye, tumors of the gastrointestinal tract, tumors of the genital system, tumors of the haematopoietic system, tumors of the mammary gland, tumors of the nervous system, tumors of the respiratory system, tumors of the skeleton, tumors of the skin, tumors of the soft tissues, and tumors of the urinary outflow system.

In a third embodiment of the first aspect which is also an embodiment of the first embodiment of the first aspect the nucleic acid molecule is for use in a method for the treatment and/or prevention of a respiratory disease, wherein the respiratory disease is selected from the group of chronic obstructive pulmonary disease.

In a fourth embodiment of the first aspect which is also an embodiment of the first embodiment of the first aspect, the nucleic acid molecule is for use in a method for the treatment and/or prevention of pain, wherein pain is selected from the group of acute pain, chronic pain and neuropathic pain.

In a fifth embodiment of the first aspect which is also an embodiment of the first embodiment of the first aspect, the nucleic acid molecule is for use in a method for the treatment and/or prevention of a neurodegenrative disorder, wherein the neurodegenerative disorder is selected from the group of Alzheimer's disease and/or Parkinson's disease.

In a sixth embodiment of the first aspect which is also an embodiment of the first to the fifth embodiment of the first aspect the nucleic acid molecule is selected from the group comprising type A nucleic acids, type B nucleic acids, type C nucleic acids, type D nucleic acids and nucleic acids having a nucleic acid sequence according to any of SEQ.ID.No. 73 to 79 and SEQ.ID.No. 207

In a seventh embodiment of the first aspect which is also an embodiment of the sixth embodiment of the first aspect, the nucleic acid molecule is a type A nucleic acid molecule, whereby the type A nucleic acid moleculecomprises in 5′->3′ direction a first terminal stretch of nucleotides, a central stretch of nucleotides and a second terminal stretch of nucleotides, whereby
the first terminal stretch of nucleotides and the second terminal stretch of nucleotides optionally hybridize with each other, whereby upon hybridization a double-stranded structure is formed,
the first terminal stretch of nucleotides comprises five to nine nucleotides,
the central stretch of nucleotides comprises a nucleotide sequence of GUCCGAUUGGCGGCACCCUUGCGGGACUGGG
the second terminal stretch of nucleotides comprises five to nine nucleotides.

In an eighth embodiment of the first aspect which is also an embodiment of the sixth embodiment of the first aspect, the nucleic acid moleculecomprises in 5′->3′ direction a second terminal stretch of nucleotides, a central stretch of nucleotides and a first terminal stretch of nucleotides, whereby

the first terminal stretch of nucleotides and the second terminal stretch of nucleotides optionally hybridize with each other, whereby upon hybridization a double-stranded structure is formed,
the first terminal stretch of nucleotides comprises five to nine nucleotides,
the central stretch of nucleotides comprises a nucleotide sequence of GUCCGAUUGGCGGCACCCUUGCGGGACUGGG
the second terminal stretch of nucleotides comprises five to nine nucleotides.

In a ninth embodiment of the first aspect which is also an embodiment of the seventh to the eighth embodiment of the first aspect, the central stretch is essential for bindung to C5a.

In a tenth embodiment of the first aspect which is also an embodiment of the seventh to the ninth embodiment of the first aspect
the double-stranded structure consists of five to nine basepairs.

In an eleventh embodiment of the first aspect which is also an embodiment of the seventh to the tenth embodiment of the first aspect, the first terminal stretch of nucleotides comprises a nucleotide sequence of 5′ X₁X₂X₃GYGCX₄Y3′ and the second terminal stretch of nucleotides comprises a nucleotide sequence of 5′ GX₅GYRCX₆X₇X₈ 3′,

whereby
X₁ is A or absent,
X₂ is G or absent,
X₃ is C or absent,

### X₄ is U,

### X₅ is A,

X₆ is G or absent,
X₇ is C or absent, and
X₈ is U or absent,
or
X₁ is A or absent,
X₂ is G or absent,
X₃ is C or absent,
X₄ is absent,
X₅ is absent,
X₆ is G or absent,
X₇ is C or absent, and
X₈ is U or absent,
preferably
X₁ is absent,
X₂ is absent,
X₃ is C or absent,

### X₄ is U,

### X₅ is A,

X₆ is G or absent,
X₇ is absent, and
X₈ is absent.

In a twelfth embodiment of the first aspect which is also an embodiment of the eleventh embodiment of the first aspect, the first terminal stretch of nucleotides comprises a nucleotide sequence of 5′ X₃GYGCX₄U 3′ and the second terminal stretch of nucleotides comprises a nucleotide sequence of 5′ GX₅GYGCX₆ 3′,

whereby
X₃ is C or absent,

### X₄ is U,

### X₅ is A, and

X₆ is G or absent.

In a 13th embodiment of the first aspect which is also an embodiment of the seventh ti the twelfth embodiment of the first aspect,
the central stretch of nucleotides comprises a first substretch of nucleotides and a second substretch of nucleotides and the first substretch of nucleotides and the second substrech of nucleotides can hybridize to each other whereby upon hybridization a double-stranded structure is formed.

In a 14th embodiment of the first aspect which is also an embodiment of the 13th embodiment of the first aspect,
each of the first and the second substrech of nucleotides comprises a sequence of three nucleotides and preferably the first substretch of nucleotides comprises the nucleotides at position 16 to 18 of the second stretch of nucleotides and the second substretch of nucleotides comprises the nucleotides 23 to 25 of the second stretch of nucleotides.

In a 15th embodiment of the first aspect which is also an embodiment of the 14th embodiment of the first aspect,
the sequence of three nucleotides for the first and the second substretch of nucleotides is independently CCC or GGG, under the proviso that the sequence of three nucleotides is different for the first and the second substretch.

In a 16th embodiment of the first aspect which is also an embodiment of the 13th to the 15th embodiment of the first aspect,
the first substrech of nucleotides and the second substretch of nucleotides are separated within the central stretch of nucleotides by a separating stretch of nucleotides comprising a least three nucleotides or a spacer, whereby preferably the nucleotides of the separating stretch of nucleotides are not hybridized to each other.

In a 17th embodiment of the first aspect which is also an embodiment of the 16th embodiment of the first aspect,
the separating stretch of nucleotides comprises at least three nucleotides, preferably consists of four nucleotides.

In an 18th embodiment of the first aspect which is also an embodiment of the 16th to the 17th embodiment of the first aspect,
within the separating stretch of nucleotides a minimum of two nucleotides is replaced by a spacer.

In a 19th embodiment of the first aspect which is also an embodiment of the 16th to the 18th eleventh embodiment of the first aspect, the separating stretch of nucleotides consists of a spacer.

In a 20th embodiment of the first aspect which is also an embodiment of the 16th to the 19th embodiment of the first aspect,
the spacer is a hydrophilic spacer.

In a 21st embodiment of the first aspect which is also an embodiment of the 20th embodiment of the first aspect,
the hydrophilic spacer consists of polyethylene moieties.

In a 22nd embodiment of the first aspect which is also an embodiment of the seventh to the 21st embodiment of the first aspect, the nucleic acid molecule comprises a nucleic acid sequence according to SEQ.ID.No 3, 11 to 14 and 167.

In a 23rd embodiment of the first aspect which is also an embodiment of the sixth embodiment of the first aspect, the nucleic acid molecule is a type B nucleic acid molecule, wherein the type B nucleic acid molecule comprises in 5′->3′ direction a first terminal stretch of nucleotides, a first central stretch of nucleotides Box A, a second central stretch of nucleotides Box L, a third central stretch of nucleotides Box B and a second terminal stretch of nucleotides, whereby
the first terminal stretch of nucleotides and the second stretch of nucleotides optionally hybridize with each other, whereby upon hybridization a double-stranded structure is formed,
the first terminal stretch of nucleotides comprises four to eight nucleotides,
the first central stretch of nucleotides Box A comprises a nucleotide sequence of ASACGCCGVRYAGGWC,
the second central stretch of nucleotides Box L comprises four to eleven nucleotides,
the third central stretch of nucleotides Box B comprises a nucleotide sequence of GWAGAAUSG or WAGAAUSG,
the second terminal stretch comprises four to eight nucleotides.

In a 24th embodiment of the first aspect which is also an embodiment of the 23rd embodiment of the first aspect, the arrangement of the first central stretch of nucleotides Box A, the second central stretch of nucleotides Box L and the third central stretch of nucleotides Box B in 5′->3′ direction is involved in bindung to C5a.

In a 25th embodiment of the first aspect which is also an embodiment of the 23rd to 24th embodiment of the first aspect,
the double-stranded structure consists of four to eight basepairs.

In a 26th embodiment of the first aspect which is also an embodiment of the 23rd to 25th embodiment of the first aspect,
the first terminal stretch of nucleotides and the first central stretch of nucleotides Box A are separated by one to four nucleotides.

In a 27th embodiment of the first aspect which is also an embodiment of the 23rd to the 26th embodiment of the first aspect,
the first terminal stretch of nucleotides and the first central stretch of nucleotides Box A are separated by one nucleotide, whereby preferably said one nucleotide is A.

In a 28th embodiment of the first aspect which is also an embodiment of the 23rd to the 27th embodiment of the first aspect,
the third central stretch of nucleotides Box B and the second terminal stretch of nucleotides are separated by one nucleotide, whereby preferably said one nucleotide is G.

In a 29th embodiment of the first aspect which is also an embodiment of the 23rd to the 28th embodiment of the first aspect,
the first terminal stretch of nucleotides and the first central stretch of nucleotides Box A are separated by one nucleotide and the third central stretch of nucleotides Box B and the second terminal stretch of nucleotides are separated by one nucleotide and the one nucleotide separating the first terminal stretch of nucleotides and the first central stretch of nucleotides Box A, and the one nucleotide separating the third central stretch of nucleotides Box B and the second terminal stretch of nucleotides do not hybridize to each other.

In a 30th embodiment of the first aspect which is also an embodiment of the sixth embodiment of the first aspect, the nucleic acid molecule is a B type nucleic acid molecule, wherein the type B nucleic acid molecule comprises in 5′->3′ direction a second central stretch of nucleotides, a first central stretch of nucleotides Box A, a second terminal stretch of nucleotides Box L, a third central stretch of nucleotides Box B and a first terminal stretch of nucleotides, whereby
the first terminal stretch of nucleotides and the second terminal stretch of nucleotides optionally hybridize with each other, whereby upon hybridization a double-stranded structure is formed, whereby
the first terminal stretch of nucleotides comprises four to eight nucleotides,
the first central stretch of nucleotides Box A comprises a nucleotide sequence of ASACGCCGVRYAGGWC,
the second central stretch of nucleotides Box L comprises four to eleven nucleotides,
the third central stretch of nucleotides Box B comprises a nucleotide sequence of GWAGAAUSG or WAGAAUSG,
the second terminal stretch of nucleotides comprises four to eight nucleotides.

In a 31st embodiment of the first aspect which is also an embodiment of the 30th embodiment of the first aspect, the arrangement of the first central stretch of nucleotides Box A, the second central stretch of nucleotides Box L and the third central stretch of nucleotides Box B in 5′->3′ direction is involved in bindung to C5a.

In a 32nd embodiment of the first aspect which is also an embodiment of the 30th to the 31st embodiment of the first aspect,
the double-stranded structure consists of four to eight basepairs.

In a 33rd embodiment of the first aspect which is also an embodiment of the 30th to the 32nd embodiment of the first aspect,
the second terminal stretch of nucleotides and the first central stretch of nucleotides Box A are separated by one to four nucleotides.

In a 34th embodiment of the first aspect which is also an embodiment of the 30th to the 33rd embodiment of the first aspect,
the second terminal stretch of nucleotides and the first central stretch of nucleotides Box A are separated by one nucleotide, whereby preferably said one nucleotide is A.

In a 35th embodiment of the first aspect which is also an embodiment of the 30th to the 34th embodiment of the first aspect,
the third central stretch of nucleotides Box B and the first terminal stretch of nucleotides are separated by one nucleotide, whereby preferably said one nucleotide is G.

In a 36th embodiment of the first aspect which is also an embodiment of the 30th to the 35th embodiment of the first aspect,
the second terminal stretch of nucleotides and the first central stretch of nucleotides Box A are separated by one nucleotide and the third central stretch of nucleotides Box B and the first terminal stretch of nucleotides are separated by one nucleotide and the one nucleotide separating the second terminal stretch of nucleotides and the first central stretch of nucleotides Box A, and the one nucleotide separating the third central stretch of nucleotides Box B and the first terminal stretch do not hybridize to each other.

In a 37th embodiment of the first aspect which is also an embodiment of the 23rd to the 37th embodiment of the first aspect, the first terminal stretch of nucleotides comprises a nucleotide sequence of 5′ X₁X₂SBBX₃X₄X₅ 3′ and the second terminal stretch of nucleotides comprises a nucleotide sequence of 5′ X₆X₇X₈VVSX₉X₁₀ 3′,

whereby
X₁ is G or absent,
X₂ is U or absent,

### X₃ is B,

### X₄ is Y,

### X₅ is M,

### X₆ is K,

### X₇ is G,

### X₈ is N,

X₉ is A or absent, and
X₁₀ is C or absent;

### Or

X₁ is G or absent,
X₂ is U or absent,

### X₃ is B,

### X₄ is Y,

X₅ is absent,
X₆ is absent,

### X₇ is G,

### X₈ is N,

X₉ is A or absent, and
X₁₀ is C or absent;
or
X₁ is G or absent,
X₂ is U or absent,

### X₃ is B,

X₄ is absent,

### X₅ is M,

### X₆ is K,

X₇ is absent,

### X₈ is N,

X₉ is A or absent, and
X₁₀ is C or absent;
or
X₁ is G or absent,
X₂ is U or absent,
X₃ is absent,

### X₄ is Y,

### X₅ is M,

### X₆ is K,

### X₇ is G,

X₈ is absent,
X₉ is A or absent, and
X₁₀ is C or absent;
or
X₁ is G or absent,
X₂ is U or absent,

### X₃ is B,

X₄ is absent,
X₅ is absent,
X₆ is absent,
X₇ is absent,

### X₈ is N,

X₉ is A or absent, and
X₁₀ is C or absent;
or
X₁ is G or absent,
X₂ is U or absent,
X₃ is absent,
X₄ is absent,

### X₅ is M,

### X₆ is K,

X₇ is absent,
X₈ is absent,
X₉ is A or absent, and
X₁₀ is C or absent,
or
X₁ is G or absent,
X₂ is U or absent,
X₃ is absent,

### X₄ is Y,

X₅ is absent,
X₆ is absent,

### X₇ is G,

X₈ is absent,
X₉ is A or absent, and
X₁₀ is C or absent;
or
X₁ is G or absent,
X₂ is U or absent,
X₃ is absent,
X₄ is absent,
X₅ is absent,
X₆ is absent,
X₇ is absent,
X₈ is absent,
X₉ is A or absent, and
X₁₀ is C or absent.

In a 38th embodiment of the first aspect which is also an embodiment of the 37th embodiment of the first aspect, the first terminal stretch of nucleotides comprises a nucleotide sequence of 5′ X₁X₂SSBX₃X₄X₅ 3′ and the second terminal stretch of nucleotides comprises a nucleotide sequence of 5′ X₆X₇X₈VSSX₉X₁₀ 3′,

whereby
X₁ is G or absent,
X₂ is U or absent,

### X₃ is S,

X₄ is absent,
X₅ is absent,
X₆ is absent,
X₇ is absent,

### X₈ is S,

X₉ is A or absent, and
X₁₀ is C or absent;
whereby preferably
X₁ is absent,
X₂ is absent,

### X₃ is S,

X₄ is absent,
X₅ is absent,
X₆ is absent,
X₇ is absent,

### X₈ is S,

X₉ is absent, and
X₁₀ is absent.

In a 39th embodiment of the first aspect which is also an embodiment of the 37th to the 38th embodiment of the first aspect, 39. The nucleic acid molecule according to claims 37 and 38,

whereby the first terminal stretch of nucleotides comprises a nucleotide sequence of 5′ GCUG 3′ and the second terminal stretch of nucleotides comprises a nucleotide sequence of 5′ CAGC 3′ or
whereby the first terminal stretch of nucleotides comprises a nucleotide sequence of 5′ CGCC 3′ and the second terminal stretch of nucleotides comprises a nucleotide sequence of 5′ GGCG 3′ or
whereby the first terminal stretch of nucleotides comprises a nucleotide sequence of 5′ CCGG 3′ and the second terminal stretch of nucleotides comprises a nucleotide sequence of 5′ CCGG 3′.

In a 40th embodiment of the first aspect which is also an embodiment of the 37th embodiment of the first aspect, the first terminal stretch of nucleotides comprises a nucleotide sequence of 5′ X₁X₂GCVX₃X₄X₅ 3′ and the second terminal stretch of nucleotides comprises a nucleotide sequence of 5′ X₆X₇X₈AGCX₉X₁₀ 3′,

whereby
X₁ is G or absent,
X₂ is U or absent,

### X₃ is G,

### X₄ is C,

X₅ is absent,
X₆ is absent,

### X₇ is G,

### X₈ is C,

X₉ is A or absent, and
X₁₀ is C or absent.

In a 41st embodiment of the first aspect which is also an embodiment of the 37th embodiment of the first aspect, the first terminal stretch of nucleotides comprises a nucleotide sequence of 5′ X₁X₂GCCX₃X₄X₅ 3′ and the second terminal stretch of nucleotides comprises a nucleotide sequence of 5′ X₆X₇X₈AGCX₉X₁₀ 3′,

whereby
X₁ is G or absent,
X₂ is U or absent,

### X₃ is G,

### X₄ is C,

### X₅ is C,

### X₆ is G,

### X₇ is G,

### X₈ is C,

X₉ is A or absent, and
X₁₀ is C or absent.

In a 42nd embodiment of the first aspect which is also an embodiment of the 23rd to the 41st embodiment of the first aspect,
the second nucleotide at the 5′-end of the first central stretch of nucleotides Box A is C and the penultimate nucleotide at the 3′-end of the third central stretch of nucleotides Box B is G or
the second nucleotide at the 5′-end of the first central stretch of nucleotides Box A is G and the penultimate nucleotide at the 3′-end of the third central stretch Box B is C.

In a 43rd embodiment of the first aspect which is also an embodiment of the 23rd to the 42nd embodiment of the first aspect,
the penultimate nucleotide at the 3′-end of the first central stretch of nucleotides Box A is A and the second nucleotide at the 5′-end of the third central stretch of nucleotides Box B is U or
the penultimate nucleotide at the 3′-end of the first central stretch of nucleotides Box A is U and the second nucleotide at the 5′-end of the third central stretch of nucleotides Box B is A.

In a 44th embodiment of the first aspect which is also an embodiment of the 23rd to the 43rd embodiment of the first aspect,
the first central stretch of nucleotides Box A comprises a nucleotide sequence of ASACGCCGMRYAGGWC, preferably a nucleotide sequence of ACACGCCGCGUAGGAC.

In a 45th embodiment of the first aspect which is also an embodiment of the 23rd to the 44th embodiment of the first aspect,
the third central stretch of nucleotides Box B comprises a nucleotide sequence of GUAGAAUGG or UAGAAUGG.

In a 46th embodiment of the first aspect which is also an embodiment of the 23rd to the 45th embodiment of the first aspect,
the second central stretch of nucleotides Box L comprises a first substretch of nucleotides and a second substretch of nucleotides and the first substretch of nucleotides and the second substrech of nucleotides hybridize to each other whereby upon hybridization a double-stranded structure is formed.

In a 47th embodiment of the first aspect which is also an embodiment of the 46th embodiment of the first aspect,
the sequence of the first substretch of nucleotides is C and the sequence of the second substretch of nucleotides is G or GG, or
the sequence of the first substretch of nucleotides is CC and the sequence of the second substretch of nucleotides is GG, or
the sequence of the first substretch of nucleotides is GC and the sequence of the second substretch of nucleotides is GC.

In a 48th embodiment of the first aspect which is also an embodiment of the 46th to the 47th embodiment of the first aspect,
the first substrech of nucleotides and the second substretch of nucleotides are separated within the second stretch of nucleotides by a separating stretch of nucleotides comprising a spacer or a nucleotide sequence of AAU or UUU whereby preferably the nucleotides of the separating stretch of nucleotides are not hybridized to each other.

In a 49th embodiment of the first aspect which is also an embodiment of the 46th to the 48th embodiment of the first aspect, the second central stretch Box L comprises a nucleotide sequence selected from the group of ‘GCAUUGC’, ‘CGAAGU’ and ‘CCAAUGG’.

In a 50th embodiment of the first aspect which is also an embodiment of the 23rd to the 45th embodiment of the first aspect, the second central stretch Box L comprises a nucleotide sequence of CGAAGU.

In a 51st embodiment of the first aspect which is also an embodiment of the 48th embodiment of the first aspect,
within the separating stretch of nucleotides a minimum of two nucleotides is replaced by a spacer, preferably a hydrophilic spacer, more preferably consisting of polyethylene moieties.

In a 52nd embodiment of the first aspect which is also an embodiment of the 48th to the 49th embodiment of the first aspect,
the separating stretch consists of a spacer, preferably a hydrophilic spacer, more preferably consisting of polyethylene moieties.

In a 53rd embodiment of the first aspect which is also an embodiment of the 23rd to the 52nd embodiment of the first aspect, the nucleic acid molecule comprises a nucleic acid sequence according to SEQ.ID.No 21 to 23, 26, 33, 34, 36, 37, 40, 46, 47, 168, 190, 204 to 206 and 208 to 210.

In a 54th embodiment of the first aspect which is also an embodiment of the 6th embodiment of the first aspect, the nucleic acid molecule is a type C nucleic acid molecule, wherein the type C nucleic acid molecule comprises in 5′->3′ direction a first terminal stretch of nucleotides, a central stretch of nucleotides and a second terminal stretch of nucleotides, whereby
the first terminal stretch of nucleotides and the second terminal stretch of nucleotides optionally hybridize with each other, whereby upon hybridization a double-stranded structure is formed,
the first terminal stretch of nucleotides comprises five to eight nucleotides,
the central stretch of nucleotides comprises a nucleotide sequence of GUGUUUAYUYGCUUAAUAGGGR,
the second terminal stretch of nucleotides comprises five to eight nucleotides.

In a 55th embodiment of the first aspect which is also an embodiment of the 6th embodiment of the first aspect, the nucleic acid molecule is a type C nucleic acid molecule, wherein the type C nucleic acid molecule comprises in 5′->3′ direction a second terminal stretch of nucleotides, a central stretch of nucleotides and a first terminal stretch of nucleotides, whereby
the first terminal stretch of nucleotides and the second terminal stretch of nucleotides optionally hybridize with each other, whereby upon hybridization a double-stranded structure is formed,
the first terminal stretch of nucleotides comprises five to eight nucleotides,
the central stretch of nucleotides comprises a nucleotide sequence of GUGUUUAYUYGCUUAAUAGGGR,
the second terminal stretch of nucleotides comprises five to eight nucleotides.

In a 56th embodiment of the first aspect which is also an embodiment of the 54th to the 55th embodiment of the first aspect, the central stretch is essential for bindung to C5a.

In a 57th embodiment of the first aspect which is also an embodiment of the 54th to the 56th embodiment of the first aspect, the double-stranded structure consists of five to eight base pairs.

In a 58th embodiment of the first aspect which is also an embodiment of the 54th to the 57th embodiment of the first aspect,
the first terminal and the second terminal stretch of nucleotides each and independently comprises five nucleotides.

In a 59th embodiment of the first aspect which is also an embodiment of the 54th to the 58th embodiment of the first aspect, the first terminal stretch of nucleotides comprises a nucleotide sequence of 5′ X₁X₂X₃BVGX₄M 3′ and the second terminal stretch of nucleotides comprises a nucleotide sequence of 5′ NX₅YBNX₆X₇X₈3′,

whereby
X₁ is G or absent,
X₂ is C or absent,
X₃ is B or absent,

### X₄ is G,

### X₅ is C,

X₆ is V or absent,
X₇ is G or absent,
X₈ is C or absent;
or
X₁ is G or absent,
X₂ is C or absent,
X₃ is B or absent,
X₄ is absent,
X₅ is absent,
X₆ is V or absent,
X₇ is G or absent,
X₈ is C or absent.

In a 60th embodiment of the first aspect which is also an embodiment of the 58th embodiment of the first aspect,

### X₁ is G,

### X₂ is C,

### X₃ is B,

X₄ is absent,
X₅ is absent,

### X₆ is V,

### X₇ is G,

### X₈ is C.

In a 61st embodiment of the first aspect which is also an embodiment of the 59th to the 60th embodiment of the first aspect, the first terminal stretch of nucleotides comprise a nucleotide sequence of 5′ GGGGC 3′ and the second terminal stretch of nucleotides comprise a nucleotide sequence of 5′ GCCCC 3′.

In a 62nd embodiment of the first aspect which is also an embodiment of the 54th to the 61st embodiment of the first aspect,
the central stretch of nucleotides comprises a nucleotide sequence of GUGUUUACUUGCUUAAUAGGGG.

In a 63rd embodiment of the first aspect which is also an embodiment of the 54th to the 62nd embodiment of the first aspect, the nucleic acid molecule comprises a nucleic acid sequence according to SEQ.ID.No 49, 65, 170, 188 and 189.

In a 64th embodiment of the first aspect which is also an embodiment of the 6th embodiment of the first aspect, the nucleic acid molecule is a type D nucleic acid molecule, wherein the type D nucleic acid molecule comprises in 5′->3′ direction a first terminal stretch of nucleotides, a central stretch of nucleotides and a second terminal stretch of nucleotides, whereby
the first terminal stretch of nucleotides and the second terminal stretch of nucleotides optionally hybridize with each other, whereby upon hybridization a double-stranded structure is formed,
the first terminal stretch of nucleotides comprises five to seven nucleotides,
the central stretch of nucleotides comprises a nucleotide sequence of GUUCGGACGUGGCAUGUUCCUUGAYAAACGGUUG,
the second terminal stretch of nucleotides comprises five to seven nucleotides.

In a 65th embodiment of the first aspect which is also an embodiment of the 6th embodiment of the first aspect, the nucleic acid molecule is a type D nucleic acid molecule, wherein the type D nucleic acid molecule comprises in 5′->3′ direction a second terminal stretch of nucleotides, a central stretch of nucleotides and a first terminal stretch of nucleotides,

whereby
the first terminal stretch of nucleotides and the second terminal stretch of nucleotides optionally hybridize with each other, whereby upon hybridization a double-stranded structure is formed,
the first terminal stretch comprises five to seven nucleotides,
the central stretch of nucleotides comprises a nucleotide sequence of GUUCGGACGUGGCAUGUUCCUUGAYAAACGGUUG,
the second terminal stretch of nucleotides comprises five to seven nucleotides.

In a 66th embodiment of the first aspect which is also an embodiment of the 64th to the 65th embodiment of the first aspect, the second terminal stretch is essential for bindung to C5a and/or C5.

In a 67th embodiment of the first aspect which is also an embodiment of the 64th to the 66th embodiment of the first aspect,
the double-stranded structure consists of five to seven basepairs.

In a 68th embodiment of the first aspect which is also an embodiment of the 64th to the 67th embodiment of the first aspect, the first terminal stretch of nucleotides comprise a nucleotide sequence of 5′ X₁X₂RCKGS 3′ and the second terminal stretch of nucleotides comprise a nucleotide sequence of 5′ SCMGY X₃ X₄ 3′;

whereby
X₁ is G or absent,
X₂ is U or absent,
X₃ is A or absent,
X₄ is C or absent.

In a 69th embodiment of the first aspect which is also an embodiment of the 64th to the 68th embodiment of the first aspect,
the central stretch of nucleotides comprises a nucleotide sequence of GUUCGGACGUGGCAUGUUCCUUGACAAACGGUUG.

In a 70th embodiment of the first aspect which is also an embodiment of the 64th to the 69th embodiment of the first aspect, the nucleic acid molecule comprises a nucleic acid sequence according to SEQ.ID.No 69 to 71, 197, 199, 203 and 221.

In a 71st embodiment of the first aspect which is also an embodiment of the first to the 70th embodiment of the first aspect, preferably of the 6th to the 70th embodiment of the first aspect, the nucleic acid molecule is capable of binding C5a and C5, whereby the nucleic molecule preferably not inhibits the C5 cleavage to C5a and C5b.

In a 72nd embodiment of the first aspect which is also an embodiment of the first to the 71st embodiment of the first aspect, preferably of the 6th to the 71st embodiment of the first aspect, the nucleic acid molecule is capable of binding C5a, C5, glycosylated C5a and/or glycosylated C5, whereby the C5a, C5, glycosylated C5a and/or glycosylated C5, is human C5a, C5, glycosylated C5a and/or glycosylated C5, or monkey C5a, C5, glycosylated C5a and/or glycosylated C5, preferably human C5a, C5, glycosylated C5a and/or glycosylated C5.

In a 73rd embodiment of the first aspect which is also an embodiment of the first to the 72nd embodiment of the first aspect, preferably of the 6th to the 72nd embodiment of the first aspect, and more preferably of the 71st embodiment of the first aspect the C5a has an amino acid sequence according to SEQ ID No. 1.

In a 74th embodiment of the first aspect which is also an embodiment of the first to the 72nd embodiment of the first aspect, preferably of the 6th to the 72nd embodiment of the first aspect, and more preferably of the 72nd embodiment of the first embodiment the C5 has two chains, an alpha and a beta chain, and the nucleic acid molecule is capable of binding the alpha chain of C5 whereby the alpha chain of C5 has an amino acid sequence according to SEQ ID No. 171.

In a 75th embodiment of the first aspect which is also an embodiment of the first to the 74th embodiment of the first aspect, the nucleic acid molecule comprises a modification group, wherein the excretion of the nucleic acid molecule comprising the modification group from an organism is decreased compared to a nucleic acid molecule not comprising the modification group.

In a 76th embodiment of the first aspect which is also an embodiment of the first to the 74th embodiment of the first aspect, the nucleic acid molecule comprises a modification group, wherein the nucleic acid molecule comprising the modification group has an increased retention time in an organism compared to a nucleic acid molecule not comprising modification group.

In a 77th embodiment of the first aspect which is also an embodiment of the 75th to the 76th embodiment of the first aspect, wherein the modification group is selected from the group comprising biodegradable and non-biodegradable modifications, preferably the modification group is selected from the group comprising linear poly(ethylene)glycol, branched poly(ethylene)glycol, hydroxyethyl starch, a peptide, a protein, a polysaccharide, a sterol, polyoxypropylene, polyoxyamidate, poly(2-hydroxyethyl)-L-glutamine and polyethylene glycol.

In a 78th embodiment of the first aspect which is also an embodiment of the first to the 77th embodiment of the first aspect, the modification group is a PEG moiety consisting of a straight or branched PEG, wherein the molecular weight of the PEG moiety is preferably from about 20,000 to 120,000 Da, more preferably from about 30,000 to 80,000 Da and most preferably about 40,000 Da.

In a 79th embodiment of the first aspect which is also an embodiment of the 77th embodiment of the first aspect, the modification group is a HES moiety, wherein preferably the molecular weight of the HES moiety is from about 10,000 to 200,000 Da, more preferably from about 30,000 to 170.000 Da and most preferably about 150,000 Da.

In an 80th embodiment of the first aspect which is also an embodiment of the first to the 75th to the 79th embodiment of the first aspect, the modification group is coupled to the nucleic acid molecule via a linker, whereby preferably the linker is a biodegradable linker.

In an 81st embodiment of the first aspect which is also an embodiment of the first to the 75th to the 80th embodiment of the first aspect, the modification group is coupled to the 5′-terminal nucleotide and/or the 3′-terminal nucleotide of the nucleic acid molecule and/or to a nucleotide of the nucleic acid molecule between the 5′-terminal nucleotide of the nucleic acid molecule and the 3′-terminal nucleotide of the nucleic acid molecule.

In an 82nd embodiment of the first aspect which is also an embodiment of the first to the 81th embodiment of the first aspect, preferably of the 6th to the 81st embodiment of the first aspect, the nucleotides of or the nucleotides forming the nucleic acid molecule are L-nucleotides.

In an 83rd embodiment of the first aspect which is also an embodiment of the first to the 82nd embodiment of the first aspect, preferably of the 6th to the 82nd embodiment of the first aspect, the nucleic acid molecule is an L-nucleic acid molecule.

In an 84th embodiment of the first aspect which is also an embodiment of the first to the 83rd embodiment of the first aspect, preferably of the 6th to the 83rd embodiment of the first aspect, the nucleic acid molecule comprises at least one moiety which is capable of binding C5a, whereby said moiety consists of L-nucleotides.

In an 85th embodiment of the first aspect which is also an embodiment of the first to the 84th embodiment of the first aspect, preferably of the 6th to the 84th embodiment of the first aspect, the nucleic acid molecule is for use in or is suitable for use in the manufacture of a medicament for the treatment and/or prevention of a disease.

In a second aspect which is also a first embodiment of the second aspect, the problem underlying the instant invention is solved by a pharmaceutical composition comprising a nucleic acid molecule according to any embodiment of the first aspect and optionally a further constituent, whereby the further constituent is selected from the group comprising pharmaceutically acceptable excipients, pharmaceutically acceptable carriers and pharmaceutically active agents, preferably for use in the treatment of tumor diseases and/or chronic obstructive pulmonary disease.

In a second embodiment of the second aspect which is also an embodiment of the first embodiment of the second aspect the pharmaceutical composition comprises a nucleic acid molecule according to any embodiment of the first aspect and a pharmaceutically acceptable carrier.

In a third aspect which is also a first embodiment of the third aspect, the problem underlying the instant invention is solved by the use of a nucleic acid molecule according to any embodiment of the first aspect for the manufacture of a medicament for the treatment and/or prevention of tumors or chronic obstructive pulmonary disease.

In a 86th embodiment of the first aspect and a second embodiment of the third aspect, the medicament is for use in human medicine or for use in veterinary medicine.

In a fourth aspect which is also a first embodiment of the fourth aspect, the problem underlying the instant invention is solved by the use of a nucleic acid molecule according to any embodiment of the first aspect for the manufacture of a diagnostic means.

In a third embodiment of the third aspect which is also an embodiment of the first and the second embodiment of the third aspect, the medicament is for the treatment and/or prevention of a tumor selected from the group comprising tumors of the endocrine system, tumors of the eye, tumors of the gastrointestinal tract, tumors of the genital system, tumors of the haematopoietic system, tumors of the mammary gland, tumors of the nervous system, tumors of the respiratory system, tumors of the skeleton, tumors of the skin, tumors of the soft tissues, and tumors of the urinary outflow system.

In a fourth embodiment of the third aspect which is also an embodiment of the third embodiment of the third aspect, the medicament is used or is for use in combination with a second pharmaceutically active agent, whereby the second pharmaceutically active agent is selected from the group comprising alkylating agents, antimetabolites, mitose inhibiting agents, topoisomerase inhibitors, inhibitors of the cellular signal transduction, hormones, antibodies, immune conjugates and fusion proteins.

In a fifth embodiment of the third aspect which is also an embodiment of the first embodiment of the third aspect, the medicament is for the treatment and/or prevention of chronic obstructive pulmonary disease.

In a fifth aspect which is also a first embodiment of the fifth aspect, the problem underlying the instant invention is solved by a nucleic acid molecule capable of binding C5a and/or C5, whereby nucleic acid molecule comprises in 5′->3′ direction a first terminal stretch of nucleotides, a central stretch of nucleotides and a second terminal stretch of nucleotides, whereby
the first terminal stretch of nucleotides and the second terminal stretch of nucleotides optionally hybridize with each other, whereby upon hybridization a double-stranded structure is formed,
the first terminal stretch of nucleotides comprises four to five nucleotides,
the central stretch of nucleotides comprises a nucleotide sequence of GACAGGACCAAGGUAAGGGCGGACCGAAAAACCUAG,
the second terminal stretch of nucleotides comprises four to five nucleotides.

In a second embodiment of the fifth aspect which is also an embodiment of the first embodiment of the fifth aspect the first terminal stretch of nucleotides comprises a nucleotide sequence of 5′ UGCUG 3′ and the second terminal stretch of nucleotides comprises a nucleotide sequence of 5′ CAGCA 3′.

In a third embodiment of the fifth aspect which is also an embodiment of the second to the third first embodiment of the fifth aspect the nucleic acid molecule comprises a nucleic acid sequence according to SEQ.ID.No 207.

In a sixth aspect which is also a first embodiment of the sixth aspect, the problem underlying the instant invention is solved by a nucleic acids having a nucleic acid sequence according to any of SEQ.ID.No. 190, 204 to 206, 208 to 210, and 188 to 189, 197, 199, 203 and 221.

In a fourth embodiment of the fifth aspect which is also an embodiment of the first to the third embodiment of the fifth aspect and a second embodiment of the sixth aspect which is also an embodiment of the first embodiment of the sixth aspect, the nucleic acid molecule is capable of binding C5a and C5, whereby the nucleic molecule preferably not inhibits the C5 cleavage to C5a and C5b.

In a fifth embodiment of the fifth aspect which is also an embodiment of the first to the fourth embodiment of the fifth aspect and a third embodiment of the sixth aspect which is also an embodiment of the second to the third embodiment of the sixth aspect, the nucleic acid molecule is capable of binding C5a, C5, glycosylated C5a and/or glycosylated C5, whereby the C5a, C5, glycosylated C5a and/or glycosylated C5, is human C5a, C5, glycosylated C5a and/or glycosylated C5, or monkey C5a, C5, glycosylated C5a and/or glycosylated C5, preferably human C5a, C5, glycosylated C5a and/or glycosylated C5.

In a sixth embodiment of the fifth aspect which is also an embodiment of the first to the fifth embodiment of the fifth aspect and a fourth embodiment of the sixth aspect which is also an embodiment of the first to the third embodiment of the sixth aspect, the C5a has an amino acid sequence according to SEQ ID No. 1.

In a eight embodiment of the fifth aspect which is also an embodiment of the first to the seventh embodiment of the fifth aspect and a sixth embodiment of the sixth aspect which is also an embodiment of the first to the fifth embodiment of the sixth aspect, the C5 has two chains, an alpha and a beta chain, and the nucleic acid molecule is capable of binding the alpha chain of C5 whereby the alpha chain of C5 has an amino acid sequence according to SEQ ID No. 171.

In a ninth embodiment of the fifth aspect which is also an embodiment of the first to the eighth embodiment of the fifth aspect and a seventh embodiment of the sixth aspect which is also an embodiment of the first to the sixth embodiment of the sixth aspect, the nucleic acid comprises a modification group, wherein the excretion of the nucleic acid molecule comprising the modification group from an organism is decreased compared to a nucleic acid molecule not comprising the modification group.

In a tenth embodiment of the fifth aspect which is also an embodiment of the first to the ninth embodiment of the fifth aspect and an eight fifth embodiment of the sixth aspect which is also an embodiment of the first to the seventh embodiment of the sixth aspect, the nucleic acid molecule comprises a modification group, wherein the nucleic acid molecule comprising the modification group has an increased retention time in an organism compared to a nucleic acid molecule not comprising the modification group.

In an eleventh embodiment of the fifth aspect which is also an embodiment of the first to the tenth embodiment of the fifth aspect and a ninth embodiment of the sixth aspect which is also an embodiment of the first to the eighth embodiment of the sixth aspect, the modification group is selected from the group comprising biodegradable and non-biodegradable modifications, preferably the modification group is selected from the group comprising linear poly(ethylene)glycol, branched poly(ethylene)glycol, hydroxyethyl starch, a peptide, a protein, a polysaccharide, a sterol, polyoxypropylene, polyoxyamidate, poly(2-hydroxyethyl)-L-glutamine and polyethylene glycol.

In a twelfth embodiment of the fifth aspect which is also an embodiment of the first to the eleventh embodiment of the fifth aspect and a tenth embodiment of the sixth aspect which is also an embodiment of the first to the ninth embodiment of the sixth aspect, the modification group is a PEG moiety consisting of a straight or branched PEG, wherein the molecular weight of the PEG moiety is preferably from about 20,000 to 120,000 Da, more preferably from about 30,000 to 80,000 Da and most preferably about 40,000 Da.

In a 13th embodiment of the fifth aspect which is also an embodiment of the first to the twelfth embodiment of the fifth aspect and an eleventh embodiment of the sixth aspect which is also an embodiment of the first to the tenth embodiment of the sixth aspect, the modification group is a HES moiety, wherein preferably the molecular weight of the HES moiety is from about 10,000 to 200,000 Da, more preferably from about 30,000 to 170,000 Da and most preferably about 150,000 Da.

In a 14th embodiment of the fifth aspect which is also an embodiment of the first to the 13th embodiment of the fifth aspect and a twelfth embodiment of the sixth aspect which is also an embodiment of the first to the eleventh embodiment of the sixth aspect the modification group is coupled to the nucleic acid via a linker, whereby preferably the linker is a biodegradable linker.

In a 15th embodiment of the fifth aspect which is also an embodiment of the first to the 14th embodiment of the fifth aspect and 13th embodiment of the sixth aspect which is also an embodiment of the first to the twelfth embodiment of the sixth aspect, the modification group is coupled to the 5′-terminal nucleotide and/or the 3′-terminal nucleotide of the nucleic acid and/or to a nucleotide of the nucleic acid between the 5′-terminal nucleotide of the nucleic acid and the 3′-terminal nucleotide of the nucleic acid.

In a 16th embodiment of the fifth aspect which is also an embodiment of the first to the 15th embodiment of the fifth aspect and a 14th embodiment of the sixth aspect which is also an embodiment of the first to the 13th embodiment of the sixth aspect, the nucleotides of or the nucleotides forming the nucleic acid molecule are L-nucleotides.

In a 17th embodiment of the fifth aspect which is also an embodiment of the first to the 16th embodiment of the fifth aspect and a 15th embodiment of the sixth aspect which is also an embodiment of the first to the 14th embodiment of the sixth aspect, the nucleic acid molecule is an L-nucleic acid molecule.

In an 18th embodiment of the fifth aspect which is also an embodiment of the first to the 17th embodiment of the fifth aspect and a 16th embodiment of the sixth aspect which is also an embodiment of the first to the 15th embodiment of the sixth aspect, the nucleic acid molecule comprises at least one moiety which is capable of binding C5a, whereby such moiety consists of L-nucleotides.

In a 19th embodiment of the fifth aspect which is also an embodiment of the first to the 18th embodiment of the fifth aspect and a 17th embodiment of the sixth aspect which is also an embodiment of the first to the 16th embodiment of the sixth aspect, the nucleic acid molecule is for the manufacture of a medicament for the treatment and/or prevention of a disease and/or for use in the treatment and/or prevention of disease.

In a seventh aspect which is also a first embodiment of the seventh first aspect, the problem underlying the instant invention is solved by a pharmaceutical composition comprising a nucleic acid according to any embodiment of the fifth to the seventh aspect and optionally a further constituent, whereby the further constituent is selected from the group comprising pharmaceutically acceptable excipients, pharmaceutically acceptable carriers and pharmaceutically active agents.

In a second embodiment of the seventh aspect which is also an embodiment of the first embodiment of the seventh aspect, the pharmaceutical composition comprises a nucleic acid molecule according to any embodiment of the fifth to the seventh aspect and a pharmaceutically acceptable carrier.

In an eight aspect which is also a first embodiment of the eight aspect, the problem underlying the instant invention is solved by the use of a nucleic acid molecule according to any embodiment of the fifth to the seventh aspect for the manufacture of a medicament.

In a second embodiment of the eight aspect which is also an embodiment of the first embodiment of the eight aspect, the medicament is for use in human medicine or for use in veterinary medicine.

In a ninth aspect which is also a first embodiment of the ninth aspect, the problem underlying the instant invention is solved by the use of a nucleic acid molecule according to any embodiment of the fifth to the seventh aspect for the manufacture of a diagnostic means.

In a third embodiment of the eight aspect which is also an embodiment of the first and the second embodiment of the eight aspect, the medicament is for the treatment and/or prevention of a disease or disorder selected from the group comprising autoimmune diseases, inflammatory diseases, infectious diseases, immune complex associated diseases, disease of the eye, local inflammations, shock, sarcoidosis, septic shock, haemorrhagic shock, anaphylactic shock, systemic inflammatory response syndrome, multiple organ failure, asthma, allergy, vasculitides, whereby such vasculitis is preferably arteritis temporalis, vasculitis, vascular leakage, and atherosclerosis; myocarditis, dermatomyositis, acute respiratory insufficiency, stroke, myocardial infarction, burn, local manifestations of systemic diseases, type 1 and 2 diabetes, the manifestations of diabetes, thromboembolism, glomerulonephritis, immune complex disorders, fetal rejection, adult respiratory distress syndrome, chronic obstructive pulmonary disease, pancreatitis, peritonitis, gingivitis and the secondary damages of traumai systemic inflammatory response syndrome, multiorgan failure, neurodegeneration and inflammation such as in Alzheimer's disease, neurocognitive dysfunction, and acute injuries of the central nervous system.

In a fourth embodiment of the eight aspect which is also an embodiment of the first to the third embodiment of the eight aspect, the disease is an autoimmune disease selected from the group comprising rheumatoid arthritis, ankylosing spodylitis, systemic lupus erythematosus, multiple sclerosis, psoriasis, urticaria, alopecia greata, warm and cold autoimmune hemolytic anemia, pernicious anemia, autoimmune adrenalitis, autoimmune neurodegeneration, such as chronic inflammatory demyelinating polyneuropathy and multiple sclerosis; Churg-Strauss syndrome, Cogan syndrome, CREST syndrome, pemphigus vulgaris and pemphigus foliaceus, bullous pemphigoid, polymyalgia rheumatica, polymyositis, primary biliary cirrhosis, psoriatic arthritis, rheumatic fever, sarcoidosis, Sjδrgensen syndrome, scleroderma, celiac disease, stiff-man syndrome, Takayasu arteritis, transient gluten intolerance, autoimmune uveitis, vitiligo, polychondritis, dermatitis herpetiformis or Duhring's disease, fibromyalgia, Goodpasture syndrome, Guillain-Barre syndrome, Hashimoto thyroiditis, autoimmune hepatitis, inflammatory bowel disease auch as Crohn's disease, colitis ulcerosa; myasthenia gravis, glomerulonephritis, renal fibrosis, polyarteritis nodosa, anti-phospholipid syndrome, polyglandular autoimmune syndrome, idiopatic pulmonar fibrosis, idiopathic thrombocytopenic purpura, autoimmune infertility, juvenile rheumatoid arthritis, autoimmune cardiomyopathy, rheumatic disease in the eye, rheumatic disease in the brain, rheumatic disease in the vasculature, rheumatic disease in the heart, rheumatic disease in the lung, rheumatic disease in the kidneys, rheumatic disease in the liver, rheumatic disease in the gastrointestinal tract, rheumatic disease in the spleen, rheumatic disease in the skin, rheumatic disease in the bones, rheumatic disease in the lymphatic system, rheumatic disease in the blood or other organ systems, Lambert-Eaton syndrome, lichen sclerosis, Lyme disease, Graves disease, Behcet's disease, Meniere's disease, and reactive arthritis.

In a fifth embodiment of the eight aspect which is also an embodiment of the first to the fourth embodiment of the eight aspect, the disease is an inflammatory disease selected from the group comprising inflammatory diseases of the eye and inflammatory diseases of the vasculature.

In a sixth embodiment of the eight aspect which is also an embodiment of the first to the fifth embodiment of the eight aspect, the disease is an infectious disease caused by or associated with a viruse, whereby preferably the virus is HIV, HBV, HCV or CMV, or caused by or associated with an intracellular parasite, whereby the parasite preferably is Leishmania, Rickettsia, Chlamydia, Coxiella, Plasmodium, Brucella, mycobacteria, Listeria, Toxoplasma or Trypanosoma.

In a seventh embodiment of the eight aspect which is also an embodiment of the first to the sixth embodiment of the eight aspect, the disease is an immune complex associated disease selected from the group comprising immune-complex-mediated renal diseases such as a complication of systemic erythematosus.

In an eighth embodiment of the eight aspect which is also an embodiment of the first to the seventh embodiment of the eight aspect, the disease is a disease of the eye selected from the group comprising uveitis, age-related macular degeneration (AMD), diabetic retinopathy, diabetic macular edema, retinal vessel occlusion, choroidal neovacularization, glaucoma ocular pemphigoid, keratoconjunctivitis, Stevens-Johnson syndrome, and Graves ophthalmopathy.

In a ninth embodiment of the eight aspect which is also an embodiment of the first embodiment of the eight aspect, the medicament is for the prevention and/or support and/or post-operative treatment during and/or after surgery, preferably during and/or after coronary artery bypass graft, off-pump coronary artery bypass graft, minimally invasive direct coronary artery bypass graft, percutaneous transluminal coronary angioplasty, thrombolysis, organ transplantation, brain and spinal cord surgery, reconstructive surgery or vessel clamping surgery.

In a tenth aspect which is also a first embodiment of the tenth aspect, the problem underlying the instant invention is solved by the use of a nucleic acid according to any embodiment of the fifth to the seventh aspect for the prevention of organ damage of a transplanted organ or of an organ to be transplanted or for use in prevention of treatment of transplant rejection for a transplanted organ, whereby the organ is preferably selected from the group comprising liver, kidney, intestine, lung, heart, skin, limb, cornea, Langerhans islet, bone marrow, blood vessels and pancreas.

In an eleventh aspect which is also a first embodiment of the eleventh aspect, the problem underlying the instant invention is solved by the use of a nucleic acid according to any embodiment of the fifth to the seventh aspect for the prevention of reperfusion injury of organs whereby the organ is preferably selected from the group comprising heart, spleen, bladder, pancreas, stomach, lung, liver, kidney, limbs, brain, sceletal muscle and intestine and for prevention of delayed graft function.

In a twelfth aspect which is also a first embodiment of the twelfth aspect, the problem underlying the instant invention is solved by a storage solution and/or a transport solution, preferably for storage of an organ or transport of an organ, comprising a nucleic acid according to any embodiment fifth to the seventh aspect.

The present invention is based on the surprising finding that it is the use of nucleic acids binding specifically and with high affinity to C5a can be used for the treatment and/or prevention of tumors, the treatment and/or prevention of a respiratory disease, the treatment and/or prevention of pain and/or the treatment and/or prevention of a neurodegenerative disorder.

The present invention is based on the surprising finding that it is possible to generate nucleic acids binding specifically and with high affinity to C5a, in particular the shown affinity of several individual C5a binding nucleic acids of in the picomolar could be foreseen. Nucleic acids binding specifically and with high affinity to C5a are preferably also referred to herein as the nucleic acid molecules according to the present invention, the nucleic acids according to the present invention, the inventive nucleic acids, the inventive nucleic acid molecules or C5a binding nucleic acids.

The features of the nucleic acid according to the present invention as described herein can be realised in any aspect of the present invention where the nucleic acid is used, either alone or in any combination.

Human C5a is a basic protein having the amino acid sequence according to SEQ. ID. Nos. 1.

The finding that short high affinity binding nucleic acids to human C5a could be identified, is insofar surprising as Eaton et al. (1997) observed that the generation of aptamers, i.e. D-nucleic acids binding to a target molecule, directed to a basic protein is in general very difficult because this kind of target produces a high but non-specific signal-to-noise ratio. This high signal-to-noise ratio results from the high non-specific affinity shown by nucleic acids for basic targets such as human C5a.

As outlined in more detail in the claims and example 1, the present inventors could more surprisingly identify a number of different human C5a binding nucleic acid molecules, whereby most of the nucleic acids could be characterised in terms of stretches of nucleotide which are also referred to herein as Boxes. The various human C5a binding nucleic acid molecules can be categorised based on said Boxes and some structural features and elements, respectively. The various categories thus defined are also referred to herein as types and more specifically as Type A, Type B, Type C and Type D.

All C5a binding nucleic acids comprise terminal stretches of nucleotides at their 5′- and 3′-end, the 5′-terminal stretch and the 3′-terminal stretch These stretches are also referred to as first terminal stretch and second terminal stretch of nucleotides.

Moreover all C5a binding nucleic acids comprise a central stretch of nucleotides, whereby the central stretch can comprise one nucleotide sequence (see Type A, Type Type C and Type D C5a) or can be divided in several central stretches, e.g. first, second and third central stretch of nucleotides (as shown for Type B C5a binding nucleic acids). The different central stretches are also referred to as boxes, e.g. Box A for the first central stretch of nucleotides of the Type B C5a binding nucleic acids, Box L for the second central stretch of nucleotides of the Type B C5a binding nucleic acids, and Box for the second central stretch of nucleotides of the Type B C5a binding nucleic acids. Moreover the central stretch(es) can be divided in substreches of nucleotides, e.g. the central stretch of nucleotides of Type A binding nucleic acids can be divided in a first and a second substretch of nucleotides, whereby in the case of Type A binding nucleic acids the substretches are related to each other and the substretches are linked to each other by another substretch, a linking substrech. The division of the central stretch of nucleotides into substretches or boxes allows a better characterisation of the central stretch and its features.

It is within the present invention that the nucleic acids according to the present invention or stretches thereof or any part(s) thereof can, in principle, hybridise with each other. Upon such hybridisation a double-stranded structure is formed. It will be acknowledged by the ones skilled in the art that such hybridisation may or may not occur, particularly under in vitro and/or in vivo conditions. Also, in case of such hybridisation, it is not necessarily the case that the hybridisation occurs over the entire length of the two stretches where, at least based on the rules for base pairing, such hybridisation and thus formation of a double-stranded structure may, in principle, occur. As preferably used herein, a double-stranded structure is a part of a molecule or a structure formed by two or more separate strands or two spatially separation stretches of a single strand, whereby at least one, preferably two or more base pairs exist which are base pairing preferably in accordance with the Watson-Crick base pairing rules. It will also be acknowledged by the one skilled in the art that other base pairing such as Hoogsten base pairing may exist in or form such double-stranded structure.

In a preferred embodiment the term arrangement as used herein, means the order or sequence of structural or functional feature or elements described herein in connection with the nucleic acids disclosed herein.

It will be acknowledged by the person skilled in the art that the nucleic acids according to the present invention are capable of binding to both C5a and C5. This binding characteristic arises from the fact that for the identification of the nucleic acids a moiety of C5a was used which is present in both C5a and C5. Accordingly, the nucleic acids according to the present invention are suitable for the detection of either C5a, C5 or both. Also, it will be acknowledged by the person skilled in the art that the nucleic acids according to the present invention are antagonists to both C5 and C5a. Because of this the nucleic acids according to the present invention are suitable for the treatment and prevention, respectively, of any disease which is associated with or caused by either C5a or C5 or both. The scientific rational may be taken from the prior art which establishes that C5a and C5, respectively, are involved or associated with a variety of diseases and conditions, respectively, and which is incorporated herein by reference.

The C5a binding nucleic acids presented here have been shown to recognize C5a in the context of C5 (see Example 6). Therefore it was investigated whether C5 cleavage to the anaphylatoxin C5a and C5b, which is part of the membrane attack complex (MAC) is inhibited by C5a binding nucleic acids. The MAC is the ultimate product of the complement cascade: a pore consisting of C5b-9. MAC is believed to insert into the cytoplasmic membranes of pathogens and kill them by induction of cytoplasmic leakage. The assay for determination C5a binding nucleic acids activity with regard to this question was achieved by using a complement-dependent sheep erythrocyte hemolysis test. The C5a binding nucleic acids tested, namely 182-E5, 182-E5-5′PEG, 182-D5-001, 182-A5-001, 182-E6-001 and the non-05a/C5-binding Spiegelmer ‘PoC’ showed only weakly inhibited hemolysis cleavage, whereby the effect is probably due to unspecific interactions of the Spiegelmers with some part of the complement cascade (see Example 10). The C5a binding nucleic acids tested can not inhibit MAC formation and are therefore selective antagonists of C5a only. If used as a medicament, this may be advantageous in many diseases, since the formation of the MAC that is beneficial in pathogen defense is not compromised in their presence.

It is within the present invention that the nucleic acid according to the present invention is a nucleic acid molecule. Insofar the terms nucleic acid and nucleic acid molecule are used herein in a synonymous manner if not indicated to the contrary. In one embodiment of the present application the nucleic acid and thus the nucleic acid molecule comprises a nucleic acid molecule which is characterized in that all of the consecutive nucleotides forming the nucleic acid molecule are linked with or connected to each other by one or more than one covalent bond. More specifically, each of such nucleotides is linked with or connected to two other nucleotides, preferably through phosphodiester bonds or other bonds, forming a stretch of consecutive nucleotides. In such arrangement, however, the two terminal nucleotides, i.e. preferably the nucleotide at the 5′ end and at the 3′ end, are each linked to a single nucleotide only under the proviso that such arrangement is a linear and not a circular arrangement and thus a linear rather than a circular molecule.

In another embodiment of the present application the nucleic acid and thus the nucleic acid molecule comprises at least two groups of consecutive nucleotides, whereby within each group of consecutive nucleotides each nucleotide is linked with or connected to two other nucleotides, preferably through phosphodiester bonds or other bonds, forming a stretch of consecutive nucleotides. In such arrangement, however, the two terminal nucleotides, i.e. preferably the nucleotide at the 5′ end and at the 3′ end, are each linked to a single nucleotide only. In such embodiment, the two groups of consecutive nucleotides, however, are not linked with or connected to each other through a covalent bond which links one nucleotide of one group and one nucleotide of another or the other group through a covalent bond, preferably a covalent bond formed between a sugar moiety of one of said two nucleotides and a phosphor moiety of the other of said two nucleotides or nucleosides. In an alternative embodiment, the two groups of consecutive nucleotides, however, are linked with or connected to each other through a covalent bond which links one nucleotide of one group and one nucleotide of another or the other group through a covalent bond, preferably a covalent bond formed between a sugar moiety of one of said two nucleotides and a phosphor moiety of the other of said two nucleotides or nucleosides. Preferably, the at least two groups of consecutive nucleotides are not linked through any covalent bond. In another preferred embodiment, the at least two groups are linked through a covalent bond which is different from a phosphodiester bond. In still another embodiment, the at least two groups are linked through a covalent bond which is a phosphodiester bond.

The nucleic acids according to the present invention shall also comprise nucleic acids which are essentially homologous to the particular sequences disclosed herein. The term substantially homologous shall be understood such that the homology is at least 75%, preferably 85%, more preferably 90% and most preferably more than 95%, 96%, 97%, 98% or 99%.

The actual percentage of homologous nucleotides present in the nucleic acid according to the present invention will depend on the total number of nucleotides present in the nucleic acid. The percent modification can be based upon the total number of nucleotides present in the nucleic acid.

The homology can be determined as known to the person skilled in the art. More specifically, a sequence comparison algorithm then calculates the percent sequence identity for the test sequence(s) relative to the reference sequence, based on the designated program parameters. The test sequence is preferably the sequence or nucleic acid molecule which is said to be or to be tested whether it is homologous, and if so, to what extent, to another nucleic acid molecule, whereby such another nucleic acid molecule is also referred to as the reference sequence. In an embodiment, the reference sequence is a nucleic acid molecule as described herein, more preferably a nucleic acid molecule having a sequence according to any of SEQ. ID. NOs. 3 to 40, SEQ. ID. NOs. 43 to 79, SEQ. ID. NOs. 167-170 and SEQ. ID. NOs. 188-211). Optimal alignment of sequences for comparison can be conducted, e.g., by the local homology algorithm of Smith & Waterman (Smith & Waterman, 1981) by the homology alignment algorithm of Needleman & Wunsch (Needleman & Wunsch, 1970) by the search for similarity method of Pearson & Lipman (Pearson & Lipman, 1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

One example of an algorithm that is suitable for determining percent sequence identity is the algorithm used in the basic local alignment search tool (hereinafter “BLAST”), see, e.g. Altschul et al (Altschul et al. 1990 and Altschul et al, 1997). Software for performing BLAST analyses is publicly available through the National Center for Biotechnology Information (hereinafter “NCBI”). The default parameters used in determining sequence identity using the software available from NCBI, e.g., BLASTN (for nucleotide sequences) and BLASTP (for amino acid sequences) are described in McGinnis et al (McGinnis et al, 2004).

The term inventive nucleic acid or nucleic acid according to the present invention shall also comprise those nucleic acids comprising the nucleic acids sequences disclosed herein or part thereof, preferably to the extent that the nucleic acids or said parts are involved in the binding to human C5a. Such nucleic acid is, in an embodiment, one of the nucleic acid molecules described herein, or a derivative and/or a metabolite thereof, whereby such derivative and/or metabolite are preferably a truncated nucleic acid compared to the nucleic acid molecules described herein. Truncation may be related to either or both of the ends of the nucleic acids as disclosed herein. Also, truncation may be related to the inner sequence of nucleotides of the nucleic acid, i.e. it may be related to the nucleotide(s) between the 5′ and the 3′ terminal nucleotide, respectively. Moreover, truncation shall comprise the deletion of as little as a single nucleotide from the sequence of the nucleic acids disclosed herein. Truncation may also be related to more than one stretch of the inventive nucleic acid(s), whereby the stretch can be as little as one nucleotide long. The binding of a nucleic acid according to the present invention can be determined by the ones skilled in the art using routine experiments or by using or adopting a method as described herein, preferably as described herein in the example part.

The nucleic acids according to the present invention may be either D-nucleic acids or L-nucleic acids. Preferably, the inventive nucleic acids are L-nucleic acids. In addition it is possible that one or several parts of the nucleic acid are present as D-nucleic acids or at least one or several parts of the nucleic acids are L-nucleic acids. The term “part” of the nucleic acids shall mean as little as one nucleotide. Such nucleic acids are generally referred to herein as D- and L-nucleic acids, respectively. Therefore, in a particularly preferred embodiment, the nucleic acids according to the present invention consist of L-nucleotides and comprise at least one D-nucleotide. Such D-nucleotide is preferably attached to a part different from the stretches defining the nucleic acids according to the present invention, preferably those parts thereof, where an interaction with other parts of the nucleic acid is involved. Preferably, such D-nucleotide is attached at a terminus of any of the stretches and of any nucleic acid according to the present invention, respectively. In a further preferred embodiment, such D-nucleotides may act as a spacer or a linker, preferably attaching modifications such as PEG and HES to the nucleic acids according to the present invention.

It is also within an embodiment of the present invention that each and any of the nucleic acid molecules described herein in their entirety in terms of their nucleic acid sequence(s) are limited to the particular nucleotide sequence(s). In other words, the terms “comprising” or “comprise(s)” shall be interpreted in such embodiment in the meaning of containing or consisting of.

It is also within the present invention that the nucleic acids according to the present invention are part of a longer nucleic acid whereby this longer nucleic acid comprises several parts whereby at least one such part is a nucleic acid according to the present invention, or a part thereof. The other part(s) of these longer nucleic acids can be either one or several D-nucleic acid(s) or one or several L-nucleic acid(s). Any combination may be used in connection with the present invention. These other part(s) of the longer nucleic acid either alone or taken together, either in their entirety or in a particular combination, can exhibit a function which is different from binding, preferably from binding to C5a. One possible function is to allow interaction with other molecules, whereby such other molecules preferably are different from C5a, such as, e.g., for immobilization, cross-linking, detection or amplification. In a further embodiment of the present invention the nucleic acids according to the invention comprise, as individual or combined moieties, several of the nucleic acids of the present invention. Such nucleic acid comprising several of the nucleic acids of the present invention is also encompassed by the term longer nucleic acid.

L-nucleic acids as used herein are nucleic acids consisting of L-nucleotides, preferably consisting completely of L-nucleotides.

D-nucleic acids as used herein are nucleic acids consisting of D-nucleotides, preferably consisting completely of D-nucleotides.

The terms nucleic acid and nucleic acid molecule are used herein in an interchangeable manner if not explicitly indicated to the contrary.

Also, if not indicated to the contrary, any nucleotide sequence is set forth herein in 5′→3′ direction.

As preferably used herein any position of a nucleotide is determined or referred to relative to the 5′ end of a sequence, a stretch or a substretch. Accordingly, a second nucleotide is the second nucleotide counted from the 5′ end of the sequence, stretch and substretch, respectively. Also, in accordance therewith, a penultimate nucleotide is the second nucleotide counted from the 3′ end of a sequence, stretch and substretch, respectively.

Irrespective of whether the inventive nucleic acid consists of D-nucleotides, L-nucleotides or a combination of both with the combination being e.g. a random combination or a defined sequence of stretches consisting of at least one L-nucleotide and at least one D-nucleic acid, the nucleic acid may consist of desoxyribonucleotide(s), ribonucleotide(s) or combinations thereof.

Designing the inventive nucleic acids as L-nucleic acid is advantageous for several reasons. L-nucleic acids are enantiomers of naturally occurring nucleic acids. D-nucleic acids, however, are not very stable in aqueous solutions and particularly in biological systems or biological samples due to the widespread presence of nucleases. Naturally occurring nucleases, particularly nucleases from animal cells are not capable of degrading L-nucleic acids. Because of this the biological half-life of the L-nucleic acid is significantly increased in such a system, including the animal and human body. Due to the lacking degradability of L-nucleic acid no nuclease degradation products are generated and thus no side effects arising therefrom observed. This aspect delimits the L-nucleic acid of factually all other compounds which are used in the therapy of diseases and/or disorders involving the presence of C5a. L-nucleic acids which specifically bind to a target molecule through a mechanism different from Watson Crick base pairing, or aptamers which consists partially or completely of L-nucleotides, particularly with those parts of the aptamer being involved in the binding of the aptamer to the target molecule, are also called spiegelmers.

It is also within the present invention that the inventive nucleic acids, also referred to herein as nucleic acids according to the invention, regardless whether they are present as D-nucleic acids, L-nucleic acids or D, L-nucleic acids or whether they are DNA or RNA, may be present as single-stranded or double-stranded nucleic acids. Typically, the inventive nucleic acids are single-stranded nucleic acids which exhibit defined secondary structures due to the primary sequence and may thus also form tertiary structures. The inventive nucleic acids, however, may also be double-stranded in the meaning that two strands which are complementary or partially complementary to each other are hybridised to each other. This confers stability to the nucleic acid which, in particular, will be advantageous if the nucleic acid is present in the naturally occurring D-form rather than the L-form.

In one embodiment, one or more nucleotide(s) of the nucleic acid according to the present invention can be replaced by linker or spacer molecule. In a preferred embodiment such linker or spacer is a separating stretch as defined herein. Such linker or spacer molecule is preferably a hydrophilic spacer comprising at least one, preferably a multitude of ethylene glycol moieties. Various linkers and spacers, respectively, are known to the ones skilled in the art and can be selected using the following criteria as described, e.g., by Pils and Micura (Pils and Micura, 2000). The linkers should or do not interfere with the base pairs themselves. Linker types that contain aromatic carbocycles stack on the terminal base pair and therefore are not suitable (Lewis et al., 1999). However, eythylene gylcol based or ethylene glycol derived linkers meet these requirements as they have the advantage of good water solubility and high conformational flexibility (Thomson et al, 1993; Ma et al., 1993; Durand et al. 1990). Preferably, the spacer comprises or consists of one or several ethylene glycol moieties, whereby the oxygen is replaced or substituted by a CH₂, a phosphate or sulfur.

The inventive nucleic acids may be modified. Such modifications may be related to the single nucleotide of the nucleic acid and are well known in the art. Examples for such modification are described in, among others, Venkatesan (2003); Kusser (2000); Aurup (1994); Cummins (1995); Eaton (1995); Green (1995); Kawasaki (1993); Lesnik (1993); and Miller (1993). Such modification can be a H atom, a F atom or O—CH3 group or NH2-group at the 2′ position of the individual nucleotide of which the nucleic acid consists. Also, the nucleic acid according to the present invention can comprises at least one LNA nucleotide. In an embodiment the nucleic acid according to the present invention consists of LNA nucleotides.

In an embodiment, the nucleic acids according to the present invention may be a multipartite nucleic acid. A multipartite nucleic acid as used herein, is a nucleic acid which consists of at least two nucleic acid strands. These at least two nucleic acid strands form a functional unit whereby the functional unit is a ligand to a target molecule. The at least two nucleic acid strands may be derived from any of the inventive nucleic acids by either cleaving the nucleic acid to generate two strands or by synthesising one nucleic acid corresponding to a first part of the inventive, i.e. overall nucleic acid and another nucleic acid corresponding to the second part of the overall nucleic acid. It is to be acknowledged that both the cleavage and the synthesis may be applied to generate a multipartite nucleic acid where there are more than two strands as exemplified above. In other words, the at least two nucleic acid strands are typically different from two strands being complementary and hybridising to each other although a certain extent of complementarity between the various nucleic acid parts may exist.

Finally it is also within the present invention that a fully closed, i.e. circular structure for the nucleic acids according to the present invention is realized, i.e. that the nucleic acids according to the present invention are closed, preferably through a covalent linkage, whereby more preferably such covalent linkage is made between the 5′ end and the 3′ end of the nucleic acid sequences as disclosed herein.

The present inventors have discovered that the nucleic acids according to the present invention exhibit a very favourable K_{D} value range.

A possibility to determine the binding constants of the nucleic acid molecules according to the present invention is the use of the “pull-down assay” or the analysis by surface plasmon resonance measurement as described in the examples. An appropriate measure in order to express the intensity of the binding between the individual nucleic acid molecule and to the target which is in the present case C5a, is the so-called K_{D} value which as such as well the method for its determination are known to the one skilled in the art.

The nucleic acids according to the present invention are characterized by a certain K_{D} value. Preferably, the K_{D} value shown by the nucleic acids according to the present invention is below 1 μM. A K_{D} value of about 1 μM is said to be characteristic for a non-specific binding of a nucleic acid to a target. As will be acknowledged by the ones in the art, the K_{D} value of a group of compounds such as the nucleic acids according to the present invention are within a certain range. The above-mentioned K_{D} of about 1 μM is a preferred upper limit for the K_{D} value. The preferred lower limit for the K_{D} of target binding nucleic acids can be about 10 picomolar or higher. It is within the present invention that the K_{D} values of individual nucleic acids binding to C5a is preferably within this range. Preferred ranges can be defined by choosing any first number within this range and any second number within this range. Preferred upper values are 250 nM and 100 nM, preferred lower values are 50 nM, 10 nM, 1 nM, 100 pM and 10 pM.

The nucleic acid molecules according to the present invention may have any length provided that they are still able to bind to the target molecule. It will be acknowledged in the art that there are preferred lengths of the nucleic acids according to the present inventions. Typically, the length is between 15 and 120 nucleotides. It will be acknowledged by the ones skilled in the art that any integer between 15 and 120 is a possible length for the nucleic acids according to the present invention. More preferred ranges for the length of the nucleic acids according to the present invention are lengths of about 20 to 100 nucleotides, about 20 to 80 nucleotides, about 20 to 60 nucleotides, about 20 to 50 nucleotides and about 30 to 50 nucleotides.

It is within the present invention that the nucleic acids disclosed herein comprise a moiety which preferably is a high molecular weight moiety and/or which preferably allows to modify the characteristics of the nucleic acid in terms of, among others, residence time in the animal body, preferably the human body. A particularly preferred embodiment of such modification is PEGylation and HESylation of the nucleic acids according to the present invention. As used herein PEG stands for poly(ethylene glycole) and HES for hydroxyethly starch. PEGylation as preferably used herein is the modification of a nucleic acid according to the present invention whereby such modification consists of a PEG moiety which is attached to a nucleic acid according to the present invention. HESylation as preferably used herein is the modification of a nucleic acid according to the present invention whereby such modification consists of a HES moiety which is attached to a nucleic acid according to the present invention. The modifications such as linear poly(ethylene)glycol, branched poly(ethylene)glycol, hydroxyethyl starch, a peptide, a protein, a polysaccharide, a sterol, polyoxypropylene, polyoxyamidate, poly(2-hydroxyethyl)-L-glutamine and polyethylene glycol as well as the process of modifying a nucleic acid using such modifications, is described in European patent application EP 1 306 382, the disclosure of which is herewith incorporated in its entirety by reference.

Preferably, the molecular weight of a modification consisting of or comprising a high molecular weight moiety is about from 2,000 to 250,000 Da, preferably 20,000 to 200,000 Da. In the case of PEG being such high molecular weight moiety the molecular weight is preferably 20,000 to 120,000 Da, more preferably 40,000 to 80,000 Da. In the case of HES being such high molecular weight moiety the molecular weight is preferably 20,000 to 200,000 Da, more preferably 40,000 to 150,000 Da. The process of HES modification is, e.g., described in German patent application DE 1 2004 006 249.8 the disclosure of which is herewith incorporated in its entirety by reference.

It is within the present invention that either of PEG and HES may be used as either a linear or branched from as further described in the patent applications WO2005074993 and PCT/EP02/11950. Such modification can, in principle, be made to the nucleic acid molecules of the present invention at any position thereof. Preferably such modification is made either to the 5′-terminal nucleotide, the 3′-terminal nucleotide and/or any nucleotide between the 5′ nucleotide and the 3′ nucleotide of the nucleic acid molecule.

The modification and preferably the PEG and/or HES moiety can be attached to the nucleic acid molecule of the present invention either directly or through a linker. It is also within the present invention that the nucleic acid molecule according to the present invention comprises one or more modifications, preferably one or more PEG and/or HES moiety. In an embodiment the individual linker molecule attaches more than one PEG moiety or HES moiety to a nucleic acid molecule according to the present invention. The linker used in connection with the present invention can itself be either linear or branched. This kind of linkers are known to the ones skilled in the art and are further described in the patent applications WO2005074993 and WO2003035665.

In a preferred embodiment the linker is a biodegradable linker. The biodegradable linker allows to modify the characteristics of the nucleic acid according to the present invention in terms of, among other, residence time in the animal body, preferably in the human body, due to release of the modification from the nucleic acid according to the present invention. Usage of a biodegradable linker may allow a better control of the residence time of the nucleic acid according to the present invention. A preferably embodiment of such biodegradable linker are biodegradable linker as described in but not limited to the international patent applications WO2006/052790, WO2008/034122, WO2004/092191 and WO2005/099768, whereby in the international patent applications WO2004/092191 and WO2005/099768, the linker is part of a polymeric oligonucleotide prodrug that consists of one or two modifications as described herein, a nucleic acid molecule and the biodegradable linker in between.

It is within the present invention that the modification or modification group is a biodegradable modification, whereby the biodegradable modification can be attached to the nucleic acid molecule of the present invention either directly or through a linker. The biodegradable modification allows to modify the characteristics of the nucleic acid according to the present invention in terms of, among other, residence time in the animal body, preferably in the human body, due to release of the modification from the nucleic acid according to the present invention. Usage of biodegradable modification may allow a better control of the residence time of the nucleic acid according to the present invention. A preferably embodiment of such biodegradable modification is biodegradable as described in but not restricted to the international patent applications WO2002/065963, WO2003/070823, WO2004/113394 and WO2000/41647, in WO2000/41647 preferably page 18, line 4 to 24.

Beside the modifications as described supra, other modifications can be used to modify the characteristics of the nucleic acids according to the present invention, whereby such modifications are selected from the group of proteins, lipids such as cholesterol and sugar chains such as amylase, dextran etc.

Without wishing to be bound by any theory, it seems that by modifying the nucleic acids according to the present invention with high molecular weight moiety such as a polymer and more particularly the polymers disclosed herein, which are preferably physiologically acceptable, the excretion kinetic is changed. More particularly, it seems that due to the increased molecular weight of such modified inventive nucleic acids and due to the nucleic acids not being subject to metabolism particularly when in the L form, excretion from an animal body, preferably from a mammalian body and more preferably from a human body is decreased. As excretion typically occurs via the kidneys, the present inventors assume that the glomerular filtration rate of the thus modified nucleic acid is significantly reduced compared to the nucleic acids not having this kind of high molecular weight modification which results in an increase in the residence time in the body. In connection therewith it is particularly noteworthy that, despite such high molecular weight modification the specificity of the nucleic acid according to the present invention is not affected in a detrimental manner. Insofar, the nucleic acids according to the present invention have surprising characteristics—which normally cannot be expected from pharmaceutically active compounds—such that a pharmaceutical formulation providing for a sustained release is not necessarily required to provide for a sustained release. Rather the nucleic acids according to the present invention in their modified form comprising a high molecular weight moiety, can as such already be used as a sustained release-formulation. Insofar, the modification(s) of the nucleic acid molecules as disclosed herein and the thus modified nucleic acid molecules and any composition comprising the same may provide for a distinct, preferably controlled pharmacokinetics and biodistribution thereof. This also includes residence time in circulation and distribution to tissues. Such modifications are further described in the patent application WO2003035665.

However, it is also within the present invention that the nucleic acids disclosed herein do not comprise any modification and particularly no high molecular weight modification such as PEGylation or HESylation. Such embodiment is particularly preferred when the nucleic acid shows preferential distribution to any target organ or tissue in the body or when a fast clearance of the nucleic acids from the body after administration is desired. Nucleic acids as disclosed herein with a preferential distribution profile to any target organ or tissue in the body would allow establishment of effective local concentrations in the target tissue while keeping systemic concentration of the nucleic acids low. This would allow the use of low doses which is not only beneficial from an economic point of view, but also reduces unnecessary exposure of other tissues to the nucleic acid agent, thus reducing the potential risk of side effects. Fast clearance of the nucleic acids as disclosed herein from the body after administration might be desired in case of in vivo imaging or specific therapeutic dosing requirements using the nucleic acids or medicaments comprising the same, each according to the present invention.

The inventive nucleic acids, which are also referred to herein as the nucleic acids according to the present invention, and/or the antagonists according to the present invention may be used for the generation or manufacture of a medicament. Such medicament or a pharmaceutical composition according to the present invention contains at least one of the inventive nucleic acids, optionally together with further pharmaceutically active compounds, whereby the inventive nucleic acid preferably acts as pharmaceutically active compound itself. Such medicaments comprise in preferred embodiments at least a pharmaceutically acceptable carrier. Such carrier may be, e.g., water, buffer, PBS, glucose solution, preferably a 5% glucose salt balanced solution, starch, sugar, gelatine or any other acceptable carrier substance. Such carriers are generally known to the one skilled in the art. It will be acknowledged by the person skilled in the art that any embodiments, use and aspects of or related to the medicament of the present invention is also applicable to the pharmaceutical composition of the present invention and vice versa.

The indication, diseases and disorders for the treatment and/or prevention of which the nucleic acids, the pharmaceutical compositions and medicaments in accordance with or prepared in accordance with the present invention result from the involvement, either direct or indirect, of C5a in the respective pathogenetic mechanism.

The local release of C5a at sites of inflammation results in powerful pro-inflammatory stimuli. Thus, neutralization of C5a might be beneficial in many acute or chronic conditions, such as immune complex associated diseases in general (Heller et al., 1999); neurodegeneration and inflammation, e.g. in Alzheimer's disease (Bonifati & Kishore, 2007), where the complement C5a receptor antagonist PMX205 improved behavioral parameters and a reduction of pathological markers such as fibrillar deposits and activated glia (Fonseca et al. 2009). Other inflammatory diseases with C5a involvement are systemic lupus erythematosus (Jacob et al. 2010a; Jacob et al. 2010b), asthma (Kohl, 2001); secondary damages of trauma (Yao et al. 1998); septic shock (Huber-Lang et al., 2001); systemic inflammtory response syndrome (SIRS); multiorgan failure (MOF); acute respiratory distress syndrome (ARDS); inflammatory bowel syndrome (IBD) (Woodruff et al., 2003); immune-complex-mediated renal disease (Wang, 2006), e.g. as a complication of systemic lupus erythematosus (Manderson et al, 2004); infections; severe burns (Piccolo et al., 1999); reperfusion injury of organs such as heart, spleen, bladder, pancreas, stomach, lung, liver, kidney, limbs, brain, sceletal muscle or intestine (Riley et al., 2000) that may lead amongst others to delayed graft function (Lewis et al, 2008); psoriasis (Bergh et al., 1993); myocarditis; multiple sclerosis (Muller-Ladner et al., 1996); paroxysmal nocturnal hemoglobinuria (PNH), hemolysis, thromboembolism (Hillmern et al. 2007) and rheumatoid arthritis (RA) (Woodruff et al., 2002). Complement C5a has also been found in elevated amounts in drusen in age-related macular degeneration and it has been shown to lead to increased VEGF-expression and to promote choroidal neovascularization that may lead to vision impairment and loss (Nozaki et al, 2006).

Activation of the complement system has also been shown to raise susceptibility to develop cerebral malaria in a mouse model. C5a or C5a receptor blockade using serum from mice immunized with theses molecules conferred resistance to cerebral malaria. Therefore, blocking the C5 C5a axis may be beneficial in the prevention of developing malaria, especially cerebral malaria in humans (Patel et al. 2008).

An expert review on possible and already pursued complement-targeted therapies recently appeared in Nature biotechnology (Ricklin & Lambris, 2007).

Of course, because the C5a binding nucleic acids according to the present invention interact with or bind to human C5a, a skilled person will generally understand that the C5a binding nucleic acids according to the present invention can easily be used for the treatment, prevention and/or diagnosis of any disease of humans and animals as described herein. In connection therewith, it is to be acknowledged that the nucleic acid molecules according to the present invention can be used for the treatment and prevention of any of the diseases, disorder or condition described herein, irrespective of the mode of action underlying such disease, disorder and condition.

In the following, and without wishing to be bound by any theory, the rational for the use of the nucleic acid molecules according to the present invention in connection with the various diseases, disorders and conditions is provided, thus rendering the claimed therapeutic, preventive and diagnostic applicability of the nucleic acid molecules according to the present invention plausible. In order to avoid any unnecessary repetition, it should be acknowledged that due to the involvement of the C5a-C5a receptor axis as outlined in connection therewith said axis may be addressed by the nucleic acid molecules according to the present invention such that the claimed therapeutic, preventive and diagnostic effect is achieved. It should furthermore be acknowledged that the particularities of the diseases, disorders and conditions, of the patients and any detail of the treatment regimen described in connection therewith, may be subject to preferred embodiments of the instant application.

Myloid-derived suppressor (abbr. MDS) cells were originally observed in cancer patient >30 years ago, their role as spoilers of anti-tumor immunity is only now being appreciated. A heterogeneous population of normal myeloid cells trapped in intermediate stages of differentiation, MDS cells accumulate in the blood, lymph nodes and at tumor sites in virtually all cancer patients. In healthy individuals, these cells differentiate into macrophages, dendritic cells and neutrophils, but the tumors secrete a range of factors that disrupt differentiation of immune progenitor cells (Ostrand-Rosenberg, 2008). As shown for isolated MSD cells from the peripheral blood and the spleens of healthy mice, the MDS cells express the C5a receptor on their surface to a similar extent—an abundant expression—to that of their mature counterparts granulocytes and monocytes. As well in tumor bearing mice, the MDS cells express the C5a receptor, but the expression level is lower on the surface of tumor associated MSD cells than on MSD cells in the peripheral blood and spleen. The reason is that the C5a receptor is internalized in tumor associated cells as shown by Markiewski et al, a C5a receptor antagonist can block the function of the C5a receptor on the surface of the MSD cells and led to an impaired tumor growth (Markiewski et al. 2008).

Accordingly, disease and/or disorders and/or diseased conditions for the treatment and/or prevention of which the medicament according to the present invention may be used include, but are not limited to tumor associated diseases and/or disorders and/or diseased conditions.

In a preferred embodiment, tumor or tumour is the name for a swelling or lesion formed by an abnormal growth of cells (termed neoplastic). A tumor can be benign, pre-malignant or malignant tumor. Moreover a tumor can be a solid tumor, preferably carcininoma, sarcomaa, aoteoma, fibrosarcoma, and chondrosoma

The tumors which can in particular be treated by a medicament or a nucleic acid molecule according to the present invention are preferably those tumors which are selected from the group comprising tumors of

the endocrine system,
the eye
the gastrointestinal tract,
the genital system,
the haematopoietic system (including mixed and embryonic tumours).
the mammary gland,
the nervous system
the respiratory system,
the skeleton,
the skin
the soft tissues,
the urinary outflow system.

The group of the tumors of the endocrine system preferably comprises:
A) Tumors of the thyroid gland/parathyroid, preferably adenoma and adenocarcinoma;
B) Tumors of the suprarenal gland, preferably adenoma, adenocarcinoma and pheochromocytoma (medullosuprarenoma);
C) Tumors of the hypothalamus/hypophysis, preferably adenoma and adenocarcinoma;
D) Tumors of the endocrine pancreas, preferably insulinoma (beta cell tumor, APUDom) and Zollinger-Ellison syndrome (gastrin secernent tumor of the delta cells of the pancreas); and
E) as well as multiple endocrine neoplasias (MEN) and chemodectoma.

The group of the tumors of the eye preferably comprises:
A) Tumors of the eyelids and of the lid glands, preferably adenoma, adenocarcinoma, papilloma, histiocytoma, mast cell tumor, basal-cell tumor, melanoma, squamous-cell carcinoma, fibroma and fibrosarcoma;
B) Tumors of the conjunctiva and of the nictitating membrane, preferably squamous-cell carcinoma, haemangioma, haemangiosarcoma, adenoma, adenocarcinoma, fibrosarcoma, melanoma and papilloma; and
C) Tumors of the orbita, the optic nerve and of the eyeball, preferably retinoblastoma, osteosarcoma, mast cell tumor, meningioma, reticular cell tumor, glioma, schwannoglioma, chondroma, adenocarcinoma, squamous-cell carcinoma, plasma cell tumor, lymphoma, rhabdomyosarcoma and melanoma.

The group of tumors of the gastrointestinal tract preferably comprises:
A) Tumors of the oral cavity and of the tongue, preferably squamous-cell carcinoma, fibrosarcoma, Merkel cell tumor, inductive fibroameloblastoma, fibroma, fibrosarcoma, viral papillomatosis, idiopathic papillomatosis, nasopharyngeal polyps, leiomyosarcoma, myoblastoma and mast cell tumor;
B) Tumors of the salivary glands, preferably adenocarcinoma;
C) Tumors of the oesophagus, preferably squamous-cell carcinoma, leiomyosarcoma, fibrosarcoma, osteosarcoma, Barrett carcinoma and paraoesophageal tumors;
C) Tumors of the exocrine pancreas, preferably adenocarcinoma; and
D) Tumors of the stomach, preferably adenocarcinoma, leiomyoma, leiomyosarcoma and fibrosarcoma.

The group of tumors of the haematopoietic system preferably comprises:
A) Lymphoma, lymphatic leukemia, non-lymphactic leukemia, myeloproliferative leukemia, Hodgkin's lymphoma, Non-Hodgkin's lymphoma.

The group of tumors of the mammary glands preferably comprises:
A) fibroadenoma, adenoma, adenocarcinoma, mesenchymal tumora, carcinoma, carcinosarcoma.

The group of tumors of the nervous system preferably comprises:
A) Tumors of the skull as well as of the brain (intracranial), preferably astrocytoma, oligodendroglioma, meningioma, neuroblastoma, ganglioneuroma, ependymoma, schwannoglioma, neurofibroma, haemangioblastoma, lipoma, craniopharyngioma, teratoma and chordoma;
B) Tumors of the spinal cord and of the vertebral canal, preferably glioblastoma, meningioma, neuroblastoma, neurofibroma, osteosarcoma, chondrosarcoma, haemangiosarcoma, fibrosarcoma and multiple myeloma; and
C) Tumors of the peripheral nerves, preferably schwannoglioma, neurofibroma, neurofibrosarcoma and perineural fibroblastoma.

The group of the tumors of the respiratory system preferably comprises:
A) Tumors of the nose and nasal cavity, of the larynx and of the trachea, preferably squamous-cell carcinoma, fibrosarcoma, fibroma, lymphosarcoma, lymphoma, haemangioma, haemangiosarcoma, melanoma, mast cell tumor, osteosarcoma, chondrosarcoma, oncocytoma (rhabdomyoma), adenocarcinoma and myoblastoma; and
B) Tumors of the lung, preferably squamous-cell carcinoma, fibrosarcoma, fibroma, lymphosarcoma, lymphoma, haemangioma, haemangiosarcoma, melanoma, mast cell tumor, osteosarcoma, chondrosarcoma, oncocytoma (rhabdomyoma), adenocarcinoma, myoblastoma, small-cell carcinoma, non-small cell carcinoma, bronchial adenocarcinoma, bronchoalveolar adenocarcinoma and alveolar adenocarcinoma.

The group of the tumors of the sexual system preferably comprises:
A) Tumors of the ovaries, preferably adenoma, adenocarcinoma, cystadenoma, and undifferentiated carcinoma;
B) Tumors of the uterine, preferably leiomyoma, leiomyosarcoma, adenoma, adenocarcinoma, fibroma, fibrosarcoma and lipoma;
C) Tumors of the cervix, preferably adenocarcinoma, adenoma, leiomyosarcoma and leiomyoma;
D) Tumors of the vagina and vulva, preferably leiomyoma, leiomyosarcoma, fibroleiomyoma, fibroma, fibrosarcoma, polyps and squamous-cell carcinoma.
E) Tumors of the testicles, preferably seminoma, interstitial-cell tumor and Sertoli cell tumor;
F) Tumors of the prostate, preferably adenocarcinoma, undifferentiated carcinoma, squamous-cell carcinoma, leiomyosarcoma and transitional cell carcinoma; and
G) Tumors of the penis and the external genitals, preferably mast cell tumor and squamous-cell carcinoma.

The group of the tumors of the skeleton preferably comprises:
A) osteosarcoma, chondrosarcoma, parosteal osteosarcoma, haemangiosarcoma, synovial cell sarcoma, haemangiosarcoma, fibrosarcoma, malignant mesenchymoma, giant-cell tumor, osteoma and multilobular osteoma.

The group of tumors of the skin preferably comprises:
A) Tumors of the histiocytoma, lipoma, fibrosarcoma, fibroma, mast cell tumor, malignant melanoma, papilloma, basal-cell tumor, keratoacanthoma, haemangiopericytoma, tumors of the hair follicles, tumors of the sweat glands, tumors of the sebaceous glands, haemangioma, haemangiosarcoma, lipoma, liposarcoma, malignant fibrous histiocytoma, plasmacytoma and lymphangioma.

The group of tumors of the soft-tissues preferably comprises:
A) Tumors of the alveolar soft-tissue sarcoma, epithelioid cell sarcoma, chondrosarcoma of the soft-tissue, osteosarcoma of the soft-tissues, Ewing's sarcoma of the soft-tissues, primitive neuroectodermal tumors (PNET), fibrosarcoma, fibroma, leiomyosarcoma, leiomyoma, liposarcoma, malignant fibrous histiocytoma, malignant haemangiopericytoma, haemangioma, haemangiosarcoma, malignant mesenchymoma, malignant peripheral nerve sheath tumor (MPNST, malignant schwannoglioma, malignant melanocytic schwannoglioma, rhabdomyosarcoma, synovial sarcoma, lymphangioma and lymphangiosarcoma.

The group of tumors of the urinary outflow system preferably comprises:
A) Tumors of the kidney, preferably adenocarcinoma, transitional cell carcinoma (epithelial tumors), fibrosarcoma, chondrosarcoma (mesenchymal tumors), Wilm's tumor, nephroblastoma and embryonal nephroma (embryonal pluripotent blastoma);
B) Tumors of the ureter, preferably leiomyoma, leiomyosarcoma, fibropapilloma, transitional cell carcinoma;
C) Tumors of the urinary bladder, preferably transitional cell carcinoma, squamous-cell carcinoma, adenocarcinoma, botryoid (embryonal rhabdomyosarcoma), fibroma, fibrosarcoma, leiomyoma, leiomyosarcoma, papilloma and haemangiosarcoma; and
D) Tumors of the urethra, preferably transitional cell carcinoma, squamous-cell carcinoma and leiomyosarcoma.

The group of the mixed and embryonal tumors preferably comprises:
A) Haemangiosarcoma, thymoma and mesothelioma.

Preferably, these tumors are selected from the group comprising breast cancer, ovary carcinoma, prostate carcinoma, osteosarcoma, glioblastoma, melanoma, small-cell lung carcinoma and colorectal carcinoma.

It is within the present invention that the medicament and pharmaceutical composition, respectively, containing a nucleic acid according to the present inventors may be used for the treatment in such way.

In a further embodiment, the medicament comprises a further pharmaceutically active agent for the treatment of tumors. Such further pharmaceutically active compounds are, among others but not limited thereto, those known to antineoplastic-active substances as alkylating agents, antimetabolites, mitose inhibiting agents, topoisomerase inhibitors, inhibitors of the cellular signal transduction, hormones, antibodies, immune conjugates and fusion proteins.

The group of alkylating agents preferably comprises mechlorethamine, estramustinphosphate, melphalan, chlorambucil, bendamustin, oxazaphosphorines such as cyclophosphoamide, ifosfamide, trofosfamide, busulfan, treosulfan and thiotepa, nitrosoureas such carmustin, lomustin and nimustin, triazene such as dacarbazin, temozolomid and procarbazin, platin complexes auch as cisplatin, carboplatin, oxaliplatin and mitomycin C.

The group of antimetabolites preferably comprises antifolates such as methrotrexat and pemetrexed, purine antagonists and—analogons such as 6-mercaptopurine, 6-thioguanine, 2′-desoxycoformycine, 2-fluoro-ara-AMP, and 2-chloro-2′-desoxyadenosine, pyrimidine antagonists and—analoga such as 5-fluorouracil, capecitabene, tegafur+uracil, cytosinearabinoside, and 2′,2′-difluorodesoxycytidine, ribinucleotide reductase inhibitors such as hydroxy urea.

The group of mitose inhibitors preferably comprises vinca-alacaloids such as vincristin, vinblastin, vindesin, and vinorelbin, taxanes such as paclitaxel and docetaxel.

The group of topoisomerase inhibitors preferably comprises inhibitors of the topoisomerase I such as irinotecane and topotecane, inhibitors of topoisomerase II such as actinomycine C, antracylines, mitoxantron, amsacrin and derivates of epipodophyllotoxins.

The group of inhibitors of the cellular signal transduction preferably comprises imatinib, dasatinib, inhibitors of the EGFR-tyrosine kinase such as Gefitinib, erlotinib, sorafenib (BAY 43-9006) and sunitinib.

The group of hormones preferably comprises estrogen such as fosfetsrol, gestagnes such as medroxyprogesteronacetat and megestrolacetat, antiestrogens such as tamoxifen, droloxifen and toremifen, aromatase inhihibitors such as aminoglutethimide, formestan, anastrozol, letrozol, exemestan and fulvestrant, antiandrognes such as cyproteronacetate, flutamide and bicautamide, LHRH-agonists such as buserelini, goselerin, leuprolerin and triptorelin.

The group of antibodies, immune conjugates and fusion proteins preferably comprises monoclonal antibodies such as Rituximab, Trastuzumab, Alemtuzumab, Cetuximab, and Bevacizumab, radioactively conjugated murine antibodies such as Ibritumab tiuxetan and Tositumab, immune conjugates/oncotoxins such as Gemtuzumab Ozagamicin, toxin fusion proteins such as Denileukin Difitox,

Other pharmaceutically active compounds are, among others but not limited thereto, those known to Bleomycin, inhibitors of thymidylatsynthase such as Raltitrexed and Pemetrexed, enzymes such as L-asparaginase, Miltefosin and ANAgrelid, inhibitors of the proteasome such as Bortezomib.

Moreover, the medicament according to the present invention may be used for the treatment and/or prevention of chronic obstructive pulmonary disease.

Chronic obstructive pulmonary disease (abbr. COPD) is a lung ailment that is characterized by a persistent blockage of airflow from the lungs. It is an under-diagnosed, life-threatening lung disease that interferes with normal breathing and is not fully reversible. COPD includes a few lung diseases: the most common are chronic bronchitis and emphysema. Many people with COPD have both of these diseases. The emphysema is a damage to the air sacs at the tips of the airways what makes it hard for the body to take in the oxygen it needs. During chronic bronchitis the airways are irritated, red, and make too much sticky mucus. The walls of the airways are swollen and partly block the air from passing through.

As known neutrophils are attracted towards a C5a gradient, release superoxide radicals to kill pathogens and release beta-glucuronidase to hydrolyse complex glucuronide conjugates at the site of inflammation. However, these valuable defense mechanisms can be damaging to the body if the neutrophils are recruited to sites of pathogen-free inflammation, e.g. at reperfused sites after infaction, stroke or organ transplantation or in cases of autoimmune disease, alzheimer's disease and others that are listed below.

In turn, excessively high C5a concentrations—especially if they are not only locally elevated as they appear during sepsis—lead to an activation of the neutrophils with subsequent exhaustion and deactivation by means of reduced C5a receptor expression on the neutrophils' cell surface. This renders the patient even more vulnerable to the pathogens that persist in his body.

A C5a-binding nucleic acid that is also inhibitory for the C5a-mediated effects on its receptor (CD88) (as shown by chemotaxis assays using differentiated U937 cells) has therefore the potential to block the above named consequences of C5a signaling and could prove beneficial as part of a medicament in a number of diseases which aberrant C5a signaling is implicated in. Attenuation or blocking of C5a-mediated superoxide release, beta-glucuronidase release, chemotaxis and CD88 down-regulation were shown for one candidate. As a test system freshly prepared human polymorphonuclear leukocytes (mostly neutrophil granulocytes) from five donors were used (data not shown).

However, moreover, other disease and/or disorders and/or diseased conditions for the treatment and/or prevention of which the medicament according to the present invention may be used include, but are not limited to are autoimmune diseases such as rheumatoid arthritis (abbr. RA), ankylosing spodylitis (abbr. AS), systemic lupus erythematosus (abbr. SLE), multiple sclerosis (abbr. MS), psoriasis, alopecia greata, warm and cold autoimmune hemolytic anemia (abbr. AIHA), pernicious anemia, acute inflammatory diseases, autoimmune adrenalitis, chronic inflammatory demyelinating polyneuropathy (abbr. CIDP), Churg-Strauss syndrome, Cogan syndrome, CREST syndrome, pemphigus vulgaris and pemphigus foliaceus, bullous pemphigoid, polymyalgia rheumatica, polymyositis, primary biliary cirrhosis, pancreatitis, peritonitis, psoriatic arthritis, rheumatic fever, sarcoidosis, Sjörgensen syndrome, scleroderma, celiac disease, stiff-man syndrome, Takayasu arteritis, transient gluten intolerance, autoimmune uveitis, vitiligo, polychondritis, dermatitis herpetiformis (abbr. DH) or Duhring's disease, fibromyalgia, Goodpasture syndrome, Guillain-Barré syndrome, Hashimoto thyroiditis, autoimmune hepatitis, inflammatory bowel disease (abbr. IBD), Crohn's disease, colitis ulcerosa, myasthenia gravis, immune complex disorders, glomerulonephritis, polyarteritis nodosa, anti-phospholipid syndrome, polyglandular autoimmune syndrome, idiopatic pulmonar fibrosis, idiopathic thrombocytopenic purpura (abbr. ITP), urticaria, autoimmune infertility, juvenile rheumatoid arthritis, sarcoidosis, autoimmune cardiomyopathy, Lambert-Eaton syndrome, lichen sclerosis, Lyme disease, Graves disease, Behcet's disease, Meniere's disease, reactive arthritis (Reiter's syndrome); infections with viruses such as HIV, HBV, HCV, CMV or intracellular parasites such as Leishmania, Rickettsia, Chlamydia, Coxiella, Plasmodium, Brucella, mycobacteria, Listeria, Toxoplasma and Trypanosoma; secondary damages of trauma; local inflammation, shock, anaphylactic shock, burn, septic shock, haemorrhagic shock, systemic inflammatory response syndrome (abbr. SIRS), multiple organ failure (abbr. MOF), asthma and allergy, vasculitides such as arteritis temporalis, vasculitis, vascular leakage, and atherosclerosis; acute injuries of the central nervous system, myocarditis, dermatomyositis, gingivitis, acute respiratory insufficiency, chronic obstructive pulmonary disease, stroke, myocardial infarction, reperfusion injury, neurocognitive dysfunction, burn, inflammatory diseases of the eye such as uveitis, age-related macular degeneration (abbr. AMD), diabetic retinopathy (abbr. DR), diabetic macular edema (abbr. DME), ocular pemphigoid, keratoconjunctivitis, Stevens-Johnson syndrome, and Graves ophthalmopathy; local manifestations of systemic diseases, inflammatory diseases of the vasculature, acute injuries of the central nervous system, type 1 and 2 diabetes, the manifestations of diabetes, SLE, and rheumatic disease in the eye, brain, vasculature, heart, lung, kidneys, liver, gastrointestinal tract, spleen, skin, bones, lymphatic system, blood or other organ systems, for the prevention and/or support and/or post-operative treatment of coronary artery bypass graft (abbr. CABG), off-pump coronary artery bypass graft (abbr. OPCABG), minimally invasive direct coronary artery bypass graft (abbr. MIDCAB), percutaneous transluminal coronary angioplasty (abbr. PTCA), thrombolysis, organ transplantation, and vessel clamping surgery; for the prevention of organ damage of a transplanted organ or of an organ to be transplanted or for use of treatment of transplant rejection for transplanted organs such as liver, kidney, intestine, lung, heart, skin, limb, cornea, Langerhans islet, bone marrow, blood vessels and pancreas; fetal rejection.

The various diseases and disorders for the treatment and/or prevention of which the nucleic acids can be used, may be grouped as follows:

### Autoimmune/Inflammatory Diseases

A subgroup of autoimmune and/or inflammatory diseases are systemic autoimmune and/or inflammatory diseases. Such systemic diseases comprise
allergy
septic shock,
secondary damages of trauma
warm and cold autoimmune hemolytic anemia (abbr. AIHA),
systemic inflammatory response syndrome (abbr. SIRS),
hemorrhagic shock,
diabetes type 1,
diabetes type 2, the manifestations of diabetes,
diffuse scleroderma,
polychondritis,
polyglandular autoimmune syndrome,
rheumatoid arthritis,
systemic lupus erythematosus (abbr. SLE) and manifestations thereof,
reactive arthritis (also known as Reiter's syndrome).

A subgroup of autoimmune and/or inflammatory diseases are autoimmune and/or inflammatory diseases of the gastro-intestinal tract. Such diseases of the gastro-intestinal tract comprise
Crohn's disease,
colitis ulcerosa,
celiac disease,
transient gluten intolerance,
inflammatory bowel disease (abbr. IBD)
pancreatitis

A subgroup of autoimmune and/or inflammatory diseases are autoimmune and/or inflammatory diseases of the skin. Such diseases of the skin comprise
psoriasis,
urticaria,
dermatomyositis,
pemphigus vulgaris,
pemphigus foliaceus,
bullous pemphigoid,
Morphea/linear scleroderma,
vitiligo,
dermatitis herpetiformis (abbr. DH) or Duhring's disease,
lichen sclerosis.

A subgroup of autoimmune and/or inflammatory diseases are autoimmune and/or inflammatory diseases of the vasculature. Such diseases of the vasculature comprise vasculitides (preferably arteritis temporalis),
vasculitis,
Henoch Schönlein purpura (Chen et al. 2010)
anti-neutrophil cytoplasmic antibody (ANCA)-associated vasculitis (Chen et al. 2010)
vascular leakage,
polymyalgia rheumatica
atherosclerosis
Churg-Strauss syndrome
Takayasu arteritis
Goodpasture syndrome (mostly affecting the kidneys (glomeruli and the lungs)
glomerulonephritis
polyarteritis nodosa,
Behçet's disease

A subgroup of autoimmune and/or inflammatory diseases are autoimmune and/or inflammatory diseases of the nervous system. Such diseases of the nervous system comprise
multiple sclerosis (abbr. MS),
chronic inflammatory demyelinating polyneuropathy (abbr. CIDP),
neurocognitive dysfunction,
stiff-man syndrome,
Guillain-Barré syndrome,
myasthenia gravis,
Lambert-Eaton syndrome.

A subgroup of autoimmune and/or inflammatory diseases are muscular skeletal autoimmune and/or inflammatory diseases. Such muscular skeletal diseases comprise
rheumatoid arthritis,
rheumatic disease in the eye, brain, lung, kidneys, heart, liver, gastrointestinal tract,
spleen, skin, bones, lymphatic system, blood or other organs,
ankylosing spodylitis (abbr. AS),
sarcoidosis,
polymyalgia rheumatica,
polymyositis,
psoriatic arthritis,
rheumatic fever,
polychondritis,
fibromyalgia,
juvenile rheumatoid arthritis,
Lyme disease,
reactive arthritis (also known as Reiter's syndrome).

A subgroup of autoimmune and/or inflammatory diseases are other autoimmune and/or inflammatory diseases. Such other diseases comprise
Cogan syndrome (autoimmune eye-inflammation and hearing loss),
autoimmune adrenalitis,
immune complex disordes,
Ménière's disease,
local inflammations,
alopecia greata,
acute inflammatory diseases,
primary biliary cirrhosis,
Sjörgen's syndrome,
scleroderma,
diffuse scleroderma,
CREST syndrome,
Morphea/linear scleroderma,
autoimmune uveitis,
Hashimoto thyroiditis (autoimmune thyroid destruction),
Graves disease,
autoimmune hepatitis,
glomerulonephritis,
peritonitis,
anti-phospholipid syndrome (Chen et al. 2010),
idiopathic pulmonary fibrosis,
renal fibrosis
autoimmune infertility,
fetal rejection or miscarriage (Girardi 2008).

A subgroup of autoimmune and/or inflammatory diseases are haematological disorders. Such haematological disorders comprise
pernicious anemia (observed as a secondary damage of crohn's disease or the autoimmune destruction of intrinsic factor producing parietal cells of the stomach mucosa),
warm and cold autoimmune hemolytic anemia (abbr. AIHA),
anti-phospholipid syndrome,
idiopathic thrombocytopenic purpura (abbr. ITP).

### Diseases of the Eye

Such diseases of the eye comprise
uveitis,
age-related macular degeneration (abbr. AMD),
diabetic retinopathy (abbr. DR),
diabetic macular edema (abbr. DME),
retinal vessel occlusion,
glaucoma,
autoimmune retinal and intraocular inflammatory disease (Copland et al. 2010)
ocular pemphigoid, keratoconjunctivitis,
Stevens-Johnson syndrome,
and Graves ophthalmopathy.

### Reperfusion Injuries, Delayed Graft Function and Transplant Rejections

Such reperfusion injuries and transplant rejections comprise
stroke,
myocardial infarction,
reperfusion injuries or organ damage to transplanted organs, such as liver (Arumugam et al. 2004), kidney (Arumugam et al. 2003), intestine, lung, heart, skin, limb, cornea, islets of Langerhans, bone marrow, blood vessels and pancreas kidney damage after organ or bone marrow transplantation.

### Prevention of Transplant Rejection

Such prevention of transplant rejection comprises
transplant rejection of transplanted organs, such as liver, kidney, intestine, lung, heart, skin, limb, cornea, islets of Langerhans, bone marrow, blood vessels and pancreas.

### Cardiovascular Diseases

Such cardiovascular diseases comprise
atherosclerosis,
myocarditis,
myocardial infarction,
stroke,
Inflammatory diseases of the vasculature,
vasculitides, preferably arteritis temporalis,
vasculitis,
vascular leakage,
the manifestations of diabetes,
pre-eclempsia,
autoimmune cardiomyopathy,
for the prevention and/or support and/or post-operative treatment of coronary artery bypass graft (abbr. CABG).

### Respiratory Diseases

Such respiratory diseases comprise
asthma,
acute respiratory insufficiency,
adult respiratory distress syndrome.
chronic obstructive pulmonary disease

### Inflammatory Diseases

Such inflammatory diseases comprise
inflammatory disease of the eye,
autoimmune uveitis (Copland et al. 2010),
local manifestations of systemic diseases.

### Acute Reactions

Such acute reactions comprise
secondary damages of trauma,
shock,
burn,
anaphylactic shock,
hemorrhagic shock,
multiple organ failure (abbr. MOF),
acute injuries of the central nervous system,
acute injuries of the central nervous system.

### Pain

acute pain (Jang et al.)
chronic pain
neuropathic pain

### Neurodegenerative Disorders

Alzheimer's disease
Parkinson's disease (Farkas et al. 1998)

### Infectious Diseases

Such infectious diseases comprise
Bacterial infections, preferably
meningitis,
Lyme disease,
reactive arthritis (also known as Reiter's syndrome),
sepsis and its complications such as organ failure, cardiac dysfunction, systemic hypoperfusion, acidosis, adult respiratory distress syndrome,
infections with intracellular pathogens (Klos et al. 2009),
viral infections, preferably
HIV,
HBV,
HCV,
CMV,
viral meningitis or
intracellular parasites, preferably
Leishmania,
Rickettsia,
Chlamydia,
Coxiella,
Plasmodium, especially cerebral malaria
Brucella,
mycobacteria,
Listeria,
Toxoplasma and
Trypanosoma.

### The Nucleic Acids According to the Present Invention May Also be Used in an Intra-Operative Manner to Avoid Deleterious Effects of the Patient's Immune System, More Preferably

for the prevention and/or support and/or post-operative treatment of coronary artery bypass graft (abbr. CABG),
off-pump coronary artery bypass graft (abbr. OPCABG),
minimally invasive direct coronary artery bypass graft (abbr. MIDCAB),
percutaneous transluminal coronary angioplasty (abbr. PTCA),
thrombolysis,
organ transplantation,
brain and spinal cord surgery,
reconstructive surgery
and vessel clamping surgery;
for the prevention of organ damage of a transplanted organ or of an organ to be transplanted or
for use of treatment of transplant rejection and reperfusion injury for transplanted organs, such as liver, kidney, intestine, lung, heart, skin, limb, cornea, islets of Langerhans, bone marrow, blood vessels and pancreas.

It is within the present invention that the medicament and pharmaceutical composition, respectively, containing a nucleic acid according to the present inventors may be used for the treatment in such way.

In a further embodiment, the medicament comprises a further pharmaceutically active agent. Such further pharmaceutically active compounds are, among others but not limited thereto, those known to suppress the immune system such as calcineurin inhibitors, cyclosporin A, methotrexate, azathioprin, tacrolimus, rapamycin, chlorambucil, leflunomide, mycophenolate mofetil, brequinar, mizoribin, thalidomide, or deoxyspergualin. The further pharmaceutically active compound can be, in a further embodiment, also one of those compounds which reduce histamine production such as meclozin, clemastin, dimetinden, bamipin, ketotifen, cetirizin, lovecetirizin, cesloratadin, azelastin, mizolastin, levocabastin, terfenadin, fexofenadin, or ebastin. Such compounds can also be, but are not limited to, steroids and are preferably selected from the group comprising corticosteroids like prednisone, methylprednisolone, hydrocortisone, dexamethasone, triamcinolone, betamethasone, effervescent, or budesonide. Further, such compound can be one or several antibiotics such as, but not restricted to, aminoglycosides, β-lactam antibiotics, gyrase inhibitors, glycopeptide antibiotics, lincosamide, macrolide antibiotics, nitroimidazole derivatives, polypeptide antibiotics, sulfonamides, trimethoprim and tetracycline. Additionally, more specific anti-inflammatory or anti-angiogenic biologics can be used in combination such as IL-10, erlizumab, tolermab, rituximab, gomiliximab, basiliximab, daclizumab, HuMax-TAC, visilizumab, HuMaxCD4, clenoliximab, MAX 16H5, TNX 100, toralizumab, alemtuzumab, CY 1788, galiximab, pexelizumab, eculizumab, PMX-53, ETI 104, FG 3019, bertilimumab, 249417 (anti-factor IX) abciximab, YM 337, omalizumab, talizumab, fontolizumab, J695 (anti-IL12), I-IuMaxIL-15, mepolizumab, elsilimomab, HuDREG, anakinra, Xoma-052, adalimumab, infliximab, certolizumab, afelimomab, CytoFab, AME 527, Vapaliximab, bevacizumab, ranibizumab, vitaxin, belimumab, MLN 1202, volociximab, F200 (anti-α5β1), efalizumab, m60.11 (anti.CD11b), etanercept, onercept, natalizumab, or siplizumab, tocilizumab, ustekinumab, ABT-874. Finally, the further pharmaceutically active agent may be a modulator of the activity of any other chemokine which can be a chemokine agonist or antagonist or a chemokine receptor agonist or antagonist. Alternatively, or additionally, such further pharmaceutically active agent is a further nucleic acid according to the present invention. Alternatively, the medicament comprises at least one more nucleic acid which binds to a target molecule different from C5a or exhibits a function which is different from the one of the nucleic acids according to the present invention.

In general the C5a antagonist can be combined with inhibitors of other proinflammatory molecules or their receptors. Examples for proinflammatory molecules whose action can be attenuated in combination with the C5a antagonist are IL-1, IL-2, IL-5, IL-6, IL-8, IL-10, IL-12, IL-13, IL-15, IL-16, IL-17, IL-18, IL-23, TNF, α4β7, α5β1, BlyS, cadherin, CCR2, CD11a, CD11b, CD125, CD130, CD16, CD18, CD2, CD20, CD22, CD23, CD25, CD28, CD3, CD30, CD4, CD40, CD40L, CD44, CD45R, CD54, CD62E, CD62L, CD68, CD8, CD80, CD86, CD95, CEP, gastrin-R, C1, C1-esterase, C5, factor D, MBL, complement receptor 1, CRTH2-receptor, CTGF, E- and P-selectin, eotaxin, factor IX, FGF-20, Fgl-2, GM-CSF, GP IIb/IIIa receptor, HMG1, ICAM-1, IgE, thymocytes, IFNγ, IFNr, IP-10, MCP-1, M-CSF receptor, MIF, MMP9, PDGF-D, SDF-1, TGFβ1, tissue factor, tyrosine kinase receptor, VAP-1, VCAM-1, VEGF, VLA1, and von Willebrandt factor.

Finally, the further pharmaceutically active agent may be a modulator of the activity of any other chemokine which can be a chemokine agonist or antagonist or a chemokine receptor agonist or antagonist. Alternatively, or additionally, such further pharmaceutically active agent is a further nucleic acid according to the present invention. Alternatively, the medicament comprises at least one more nucleic acid which binds to a target molecule different from C5a or exhibits a function which is different from the one of the nucleic acids according to the present invention.

It is within the present invention that the medicament is alternatively or additionally used, in principle, for the prevention of any of the diseases disclosed in connection with the use of the medicament for the treatment of said diseases. Respective markers therefore, i.e. for the respective diseases are known to the ones skilled in the art. Preferably, the respective marker is C5a.

In one embodiment of the medicament of the present invention, such medicament is for use in combination with other treatments for any of the diseases disclosed herein, particularly those for which the medicament of the present invention is to be used.

“Combination therapy” (or “co-therapy”) includes the administration of a medicament of the invention and at least a second agent as part of a specific treatment regimen intended to provide the beneficial effect from the co-action of these therapeutic agents, i.e. the medicament of the present invention and said second agent. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

“Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to a subject a single capsule having a fixed ratio of each therapeutic agent or in multiple, single capsules for each of the therapeutic agents.

Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

Alternatively, for example, all therapeutic agents may be administered topically or all therapeutic agents may be administered by injection. The sequence in which the therapeutic agents are administered is not narrowly critical unless noted otherwise. “Combination therapy” also can embrace the administration of the therapeutic agents as described above in further combination with other biologically active ingredients. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

As outlined in general terms above, the medicament according to the present invention can be administered, in principle, in any form known to the ones skilled in the art. A preferred route of administration is systemic administration, more preferably by parenteral administration, preferably by injection. Alternatively, the medicament may be administered locally. Other routes of administration comprise intramuscular, intraperitoneal, and subcutaneous, per orum, intranasal, intratracheal or pulmonary with preference given to the route of administration that is the least invasive, while ensuring efficiency.

Parenteral administration is generally used for subcutaneous, intramuscular or intravenous injections and infusions. Additionally, one approach for parenteral administration employs the implantation of a slow-release or sustained-released systems, which assures that a constant level of dosage is maintained, that are well known to the ordinary skill in the art.

Furthermore, preferred medicaments of the present invention can be administered in intranasal form via topical use of suitable intranasal vehicles, inhalants, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would typically range from 0.01% to 15%, w/w or w/v.

The medicament of the present invention will generally comprise an effective amount of the active component(s) of the therapy, including, but not limited to, a nucleic acid molecule of the present invention, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the medicament of the present invention.

In a further aspect the present invention is related to a pharmaceutical composition. Such pharmaceutical composition comprises at least one of the nucleic acids according to the present invention and preferably a pharmaceutically acceptable vehicle. Such vehicle can be any vehicle or any binder used and/or known in the art. More particularly such binder or vehicle is any binder or vehicle as discussed in connection with the manufacture of the medicament disclosed herein. In a further embodiment, the pharmaceutical composition comprises a further pharmaceutically active agent.

The preparation of a medicament and a pharmaceutical composition will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, creams, lotions, salves, inhalants and the like. The use of sterile formulations, such as saline-based washes, by surgeons, physicians or health care workers to treat a particular area in the operating field may also be particularly useful. Compositions may also be delivered via microdevice, microparticle or sponge.

Upon formulation, a medicament will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the individual or the subject to be treated. Specific amounts of active compound required for administration depend on the judgment of the practitioner and are peculiar to each individual.

A minimal volume of a medicament required to disperse the active compounds is typically utilized. Suitable regimes for administration are also variable, but would be typified by initially administering the compound and monitoring the results and then giving further controlled doses at further intervals.

For instance, for oral administration in the form of a tablet or capsule (e.g., a gelatin capsule), the active drug component, i.e. a nucleic acid molecule of the present invention and/or any further pharmaceutically active agent, also referred to herein as therapeutic agent(s) or active compound(s) can be combined with an oral, non-toxic, pharmaceutically acceptable inert carrier such as ethanol, glycerol, water and the like. Moreover, when desired or necessary, suitable binders, lubricants, disintegrating agents, and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes, and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol, and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

The medicament of the invention can also be administered in such oral dosage forms as timed release and sustained release tablets or capsules, pills, powders, granules, elixirs, tinctures, suspensions, syrups and emulsions. Suppositories are advantageously prepared from fatty emulsions or suspensions.

The pharmaceutical composition or medicament may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated.

For solid compositions, excipients include pharmaceutical grades of mannitol, lactose, starch, magnesium stearate, sodium saccharin, talcum, cellulose, glucose, sucrose, magnesium carbonate, and the like. The active compound defined above, may be also formulated as suppositories, using for example, polyalkylene glycols, for example, propylene glycol, as the carrier. In some embodiments, suppositories are advantageously prepared from fatty emulsions or suspensions.

The medicaments and nucleic acid molecules, respectively, of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, what is well known to the ordinary skill in the art. For example, the nucleic acid molecules described herein can be provided as a complex with a lipophilic compound or non-immunogenic, high molecular weight compound constructed using methods known in the art. Additionally, liposomes may bear such nucleic acid molecules on their surface for targeting and carrying cytotoxic agents internally to mediate cell killing. An example of nucleic-acid associated complexes is provided in U.S. Pat. No. 6,011,020.

The medicaments and nucleic acid molecules, respectively, of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the medicaments and nucleic acid molecules, respectively, of the present invention may be coupled to a class of biodegradable polymers useful in achieving controlled release of a drag, for example, polylactic acid, polyepsilon capro lactone, polyhydroxy butyric acid, polyorthoesters, polyacetals, polydihydropyrans, polycyanoacrylates and cross linked or amphipathic block copolymers of hydrogels.

If desired, the pharmaceutical composition and medicament, respectively, to be administered may also contain minor amounts of non-toxic auxiliary substances such as wetting or emulsifying agents, pH buffering agents, and other substances such as for example, sodium acetate, and triethanolamine oleate.

The dosage regimen utilizing the nucleic acid molecules and medicaments, respectively, of the present invention is selected in accordance with a variety of factors including type, species, age, weight, sex and medical condition of the patient; the severity of the condition to be treated; the route of administration; the renal and hepatic function of the patient; and the particular aptamer or salt thereof employed. An ordinarily skilled physician or veterinarian can readily determine and prescribe the effective amount of the drug required to prevent, counter or arrest the progress of the condition.

Effective plasma levels of the nucleic acid according to the present invention preferably range from 500 fM to 500 μM in the treatment of any of the diseases disclosed herein.

The nucleic acid molecules and medicaments, respectively, of the present invention may preferably be administered in a single daily dose, every second or third day, weekly, every second week, in a single monthly dose or every third month.

It is within the present invention that the medicament as described herein constitutes the pharmaceutical composition disclosed herein.

In a further aspect the present invention is related to a method for the treatment of a subject who is need of such treatment, whereby the method comprises the administration of a pharmaceutically active amount of at least one of the nucleic acids according to the present invention. In an embodiment, the subject suffers from a disease or is at risk to develop such disease, whereby the disease is any of those disclosed herein, particularly any of those diseases disclosed in connection with the use of any of the nucleic acids according to the present invention for the manufacture of a medicament.

It is to be understood that the nucleic acid as well as the antagonists according to the present invention can be used not only as a medicament or for the manufacture of a medicament, but also for cosmetic purposes, particularly with regard to the involvement of C5a in inflamed regional skin lesions. Therefore, a further condition or disease for the treatment or prevention of which the nucleic acid, the medicament and/or the pharmaceutical composition according to the present invention can be used, is inflamed regional skin lesions.

As preferably used herein a diagnostic or diagostic agent or diagnostic means is suitable to detect, either directly or indirectly C5a, preferably C5a as described herein and more preferably C5a as described herein in connection with the various disorders and diseases described herein. The diagnostic is suitable for the detection and/or follow-up of any of the disorders and diseases, respectively, described herein. Such detection is possible through the binding of the nucleic acids according to the present invention to C5a. Such binding can be either directly or indirectly be detected. The respective methods and means are known to the ones skilled in the art. Among others, the nucleic acids according to the present invention may comprise a label which allows the detection of the nucleic acids according to the present invention, preferably the nucleic acid bound to C5a. Such a label is preferably selected from the group comprising radioactive, enzymatic and fluorescent labels. In principle, all known assays developed for antibodies can be adopted for the nucleic acids according to the present invention whereas the target-binding antibody is substituted to a target-binding nucleic acid. In antibody-assays using unlabeled target-binding antibodies the detection is preferably done by a secondary antibody which is modified with radioactive, enzymatic and fluorescent labels and bind to the target-binding antibody at its Fc-fragment. In the case of a nucleic acid, preferably a nucleic acid according to the present invention, the nucleic acid is modified with such a label, whereby preferably such a label is selected from the group comprising biotin, Cy-3 and Cy-5, and such label is detected by an antibody directed against such label, e.g. an anti-biotin antibody, an anti-Cy3 antibody or an anti-Cy5 antibody, or—in the case that the label is biotin—the label is detected by streptavidin or avidin which naturally bind to biotin. Such antibody, streptavidin or avidin in turn is preferably modified with a respective label, e.g. a radioactive, enzymatic or fluorescent label (like an secondary antibody).

In a further embodiment the nucleic acid molecules according to the invention are detected or analysed by a second detection means, wherein the said detection means is a molecular beacon. The methodology of molecular beacon is known to persons skilled in the art. In brief, nucleic acids probes which are also referred to as molecular beacons, are a reverse complement to the nucleic acids sample to be detected and hybridise because of this to a part of the nucleic acid sample to be detected. Upon binding to the nucleic acid sample the fluorophoric groups of the molecular beacon are separated which results in a change of the fluorescence signal, preferably a change in intensity. This change correlates with the amount of nucleic acids sample present.

It will be acknowledged that the detection of C5a using the nucleic acids according to the present invention will particularly allow the detection of C5a as defined herein.

In connection with the detection of C5a a preferred method comprises the following steps:
(a) providing a sample which is to be tested for the presence of C5a,
(b) providing a nucleic acid according to the present invention,
(c) reacting the sample with the nucleic acid, preferably in a reaction vessel
whereby step (a) can be performed prior to step (b), or step (b) can be preformed prior to step (a).

In a preferred embodiment a further step d) is provided, which consists in the detection of the reaction of the sample with the nucleic acid. Preferably, the nucleic acid of step b) is immobilised to a surface. The surface may be the surface of a reaction vessel such as a reaction tube, a well of a plate, or the surface of a device contained in such reaction vessel such as, for example, a bead. The immobilisation of the nucleic acid to the surface can be made by any means known to the ones skilled in the art including, but not limited to, non-covalent or covalent linkages. Preferably, the linkage is established via a covalent chemical bond between the surface and the nucleic acid. However, it is also within the present invention that the nucleic acid is indirectly immobilised to a surface, whereby such indirect immobilisation involves the use of a further component or a pair of interaction partners. Such further component is preferably a compound which specifically interacts with the nucleic acid to be immobilised which is also referred to as interaction partner, and thus mediates the attachment of the nucleic acid to the surface. The interaction partner is preferably selected from the group comprising nucleic acids, polypeptides, proteins and antibodies. Preferably, the interaction partner is an antibody, more preferably a monoclonal antibody. Alternatively, the interaction partner is a nucleic acid, preferably a functional nucleic acid. More preferably such functional nucleic acid is selected from the group comprising aptamers, spiegelmers, and nucleic acids which are at least partially complementary to the nucleic acid. In a further alternative embodiment, the binding of the nucleic acid to the surface is mediated by a multi-partite interaction partner. Such multi-partite interaction partner is preferably a pair of interaction partners or an interaction partner consisting of a first member and a second member, whereby the first member is comprised by or attached to the nucleic acid and the second member is attached to or comprised by the surface. The multi-partite interaction partner is preferably selected from the group of pairs of interaction partners comprising biotin and avidin, biotin and streptavidin, and biotin and neutravidin. Preferably, the first member of the pair of interaction partners is biotin.

A preferred result of such method is the formation of an immobilised complex of C5a and the nucleic acid, whereby more preferably said complex is detected. It is within an embodiment that from the complex the C5a is detected.

A respective detection means which is in compliance with this requirement is, for example, any detection means which is specific for that/those part(s) of the C5a. A particularly preferred detection means is a detection means which is selected from the group comprising nucleic acids, polypeptides, proteins and antibodies, the generation of which is known to the ones skilled in the art.

The method for the detection of C5a also comprises that the sample is removed from the reaction vessel which has preferably been used to perform step c).

The method comprises in a further embodiment also the step of immobilising an interaction partner of C5a on a surface, preferably a surface as defined above, whereby the interaction partner is defined as herein and preferably as above in connection with the respective method and more preferably comprises nucleic acids, polypeptides, proteins and antibodies in their various embodiments. In this embodiment, a particularly preferred detection means is a nucleic acid according to the present invention, whereby such nucleic acid may preferably be labelled or non-labelled. In case such nucleic acid is labelled it can directly or indirectly be detected. Such detection may also involve the use of a second detection means which is, preferably, also selected from the group comprising nucleic acids, polypeptides, proteins and embodiments in the various embodiments described herein. Such detection means are preferably specific for the nucleic acid according to the present invention. In a more preferred embodiment, the second detection means is a molecular beacon. Either the nucleic acid or the second detection means or both may comprise in a preferred embodiment a detection label.

The detection label is preferably selected from the group comprising biotin, a bromo-desoxyuridine label, a digoxigenin label, a fluorescence label, a UV-label, a radio-label, and a chelator molecule. Alternatively, the second detection means interacts with the detection label which is preferably contained by, comprised by or attached to the nucleic acid. Particularly preferred combinations are as follows:
the detection label is biotin and the second detection means is an antibody directed against biotin, or wherein
the detection label is biotin and the second detection means is an avidin or an avidin carrrying molecule, or wherein
the detection label is biotin and the second detection means is a streptavidin or a stretavidin carrying molecule, or wherein
the detection label is biotin and the second detection means is a neutravidin or a neutravidin carrying molecule, or
wherein the detection label is a bromo-desoxyuridine and the second detection means is an antibody directed against bromo-desoxyuridine, or wherein
the detection label is a digoxigenin and the second detection means is an antibody directed against digoxigenin, or wherein
the detection label is a chelator and the second detection means is a radio-nuclide, whereby it is preferred that said detection label is attached to the nucleic acid. It is to be acknowledged that this kind of combination is also applicable to the embodiment where the nucleic acid is attached to the surface. In such embodiment it is preferred that the detection label is attached to the interaction partner.

Finally, it is also within the present invention that the second detection means is detected using a third detection means, preferably the third detection means is an enzyme, more preferably showing an enzymatic reaction upon detection of the second detection means, or the third detection means is a means for detecting radiation, more preferably radiation emitted by a radio-nuclide. Preferably, the third detection means is specifically detecting and/or interacting with the second detection means.

Also in the embodiment with an interaction partner of C5a being immobilised on a surface and the nucleic acid according to the present invention is preferably added to the complex formed between the interaction partner and the C5a, the sample can be removed from the reaction, more preferably from the reaction vessel where step c) and/or d) are preformed.

In an embodiment the nucleic acid according to the present invention comprises a fluorescence moiety and whereby the fluorescence of the fluorescence moiety is different upon complex formation between the nucleic acid and C5a and free C5a.

In a further embodiment the nucleic acid is a derivative of the nucleic acid according to the present invention, whereby the derivative of the nucleic acid comprises at least one fluorescent derivative of adenosine replacing adenosine. In a preferred embodiment the fluorescent derivative of adenosine is ethenoadenosine.

In a further embodiment the complex consisting of the derivative of the nucleic acid according to the present invention and the C5a is detected using fluorescence.

In an embodiment of the method a signal is created in step (c) or step (d) and preferably the signal is correlated with the concentration of C5a in the sample.

In a preferred aspect, the assays may be performed in 96-well plates, where components are immobilized in the reaction vessels as described above and the wells acting as reaction vessels.

The inventive nucleic acid may further be used as starting material for drug design. Basically there are two possible approaches. One approach is the screening of compound libraries whereas such compound libraries are preferably low molecular weight compound libraries. In an embodiment, the screening is a high throughput screening. Preferably, high throughput screening is the fast, efficient, trial-and-error evaluation of compounds in a target based assay. In best case the analysis are carried by a colorimetric measurement. Libraries as used in connection therewith are known to the one skilled in the art.

Alternatively, the nucleic acid according to the present invention may be used for rational design of drugs. Preferably, rational drug design is the design of a pharmaceutical lead structure. Starting from the 3-dimensional structure of the target which is typically identified by methods such as X-ray crystallography or nuclear magnetic resonance spectroscopy, computer programs are used to search through databases containing structures of many different chemical compounds. The selection is done by a computer, the identified compounds can subsequently be tested in the laboratory.

The rational design of drugs may start from any of the nucleic acid according to the present invention and involves a structure, preferably a three dimensional structure, which is similar to the structure of the inventive nucleic acids or identical to the binding mediating parts of the structure of the inventive nucleic acids. In any case such structure still shows the same or a similar binding characteristic as the inventive nucleic acids. In either a further step or as an alternative step in the rational design of drugs the preferably three dimensional structure of those parts of the nucleic acids binding to the neurotransmitter are mimicked by chemical groups which are different from nucleotides and nucleic acids. By this mimicry a compound different from the nucleic acids can be designed. Such compound is preferably a small molecule or a peptide.

In case of screening of compound libraries, such as by using a competitive assay which are known to the one skilled in the arts, appropriate C5a analogues, C5a agonists or C5a antagonists may be found. Such competitive assays may be set up as follows. The inventive nucleic acid, preferably a spiegelmer which is a target binding L-nucleic acid, is coupled to a solid phase. In order to identify C5a analogues labelled C5a may be added to the assay. A potential analogue would compete with the C5a molecules binding to the spiegelmer which would go along with a decrease in the signal obtained by the respective label. Screening for agonists or antagonists may involve the use of a cell culture assay as known to the ones skilled in the art.

The kit according to the present invention may comprise at least one or several of the inventive nucleic acids. Additionally, the kit may comprise at least one or several positive or negative controls. A positive control may, for example, be C5a, particularly the one against which the inventive nucleic acid is selected or to which it binds, preferably, in liquid form. A negative control may, e.g., be a peptide which is defined in terms of biophysical properties similar to C5a, but which is not recognized by the inventive nucleic acids. Furthermore, said kit may comprise one or several buffers. The various ingredients may be contained in the kit in dried or lyophilised form or solved in a liquid. The kit may comprise one or several containers which in turn may contain one or several ingredients of the kit. In a further embodiment, the kit comprises an instruction or instruction leaflet which provides to the user information on how to use the kit and its various ingredients.

The pharmaceutical and bioanalytical determination of the nucleic acid according to the present invention is elementarily for the assessment of its pharmacokinetic and biodynamic profile in several humours, tissues and organs of the human and non-human body. For such purpose, any of the detection methods disclosed herein or known to a person skilled in the art may be used. In a further aspect of the present invention a sandwich hybridisation assay for the detection of the nucleic acid according to the present invention is provided. Within the detection assay a capture probe and a detection probe are used. The capture probe is complementary to the first part and the detection probe to the second part of the nucleic acid according to the present invention. Both, capture and detection probe, can be formed by DNA nucleotides, modified DNA nucleotides, modified RNA nucleotides, RNA nucleotides, LNA nucleotides and/or PNA nucleotides.

Hence, the capture probe comprise a sequence stretch complementary to the 5′-end of the nucleic acid according to the present invention and the detection probe comprise a sequence stretch complementary to the 3′-end of the nucleic acid according to the present invention. In this case the capture probe is immobilised to a surface or matrix via its 5′-end whereby the capture probe can be immobilised directly at its 5′-end or via a linker between of its 5′-end and the surface or matrix. However, in principle the linker can be linked to each nucleotide of the capture probe. The linker can be formed by hydrophilic linkers of skilled in the art or by D-DNA nucleotides, modified D-DNA nucleotides, D-RNA nucleotides, modified D-RNA nucleotides, D-LNA nucleotides, PNA nucleotides, L-RNA nucleotides, L-DNA nucleotides, modified L-RNA nucleotides, modified L-DNA nucleotides and/or L-LNA nucleotides.

Alternatively, the capture probe comprises a sequence stretch complementary to the 3′-end of the nucleic acid according to the present invention and the detection probe comprise a sequence stretch complementary to the 5′-end of the nucleic acid according to the present invention. In this case the capture probe is immobilised to a surface or matrix via its 3′-end whereby the capture probe can be immobilised directly at its 3′-end or via a linker between of its 3′-end and the surface or matrix. However, in principle, the linker can be linked to each nucleotide of the sequence stretch that is complementary to the nucleic acid according to the present invention. The linker can be formed by hydrophilic linkers of skilled in the art or by D-DNA nucleotides, modified D-DNA nucleotides, D-RNA nucleotides, modified D-RNA nucleotides, D-LNA nucleotides, PNA nucleotides, L-RNA nucleotides, L-DNA nucleotides, modified L-RNA nucleotides, modified L-DNA nucleotides and/or L-LNA nucleotides.

The number of nucleotides of the capture and detection probe that may hybridise to the nucleic acid according to the present invention is variable and can be dependant from the number of nucleotides of the capture and/or the detection probe and/or the nucleic acid according to the present invention itself. The total number of nucleotides of the capture and the detection probe that may hybridise to the nucleic acid according to the present invention should be maximal the number of nucleotides that are comprised by the nucleic acid according to the present invention. The minimal number of nucleotides (2 to 10 nucleotides) of the detection and capture probe should allow hybridisation to the 5′-end or 3′-end, respectively, of the nucleic acid according to the present invention. In order to realize high specificity and selectivity between the nucleic acid according to the present invention and other nucleic acids occurring in samples that are analyzed the total number of nucleotides of the capture and detection probe should be or maximal the number of nucleotides that are comprised by the nucleic acid according to the present invention.

Moreover the detection probe preferably carries a marker molecule or label that can be detected as previously described herein. The label or marker molecule can in principle be linked to each nucleotide of the detection probe. Preferably, the label or marker is located at the 5′-end or 3′-end of the detection probe, whereby between the nucleotides within the detection probe that are complementary to the nucleic acid according to the present invention, and the label a linker can be inserted. The linker can be formed by hydrophilic linkers of skilled in the art or by D-DNA nucleotides, modified D-DNA nucleotides, D-RNA nucleotides, modified D-RNA nucleotides, D-LNA nucleotides, PNA nucleotides, L-RNA nucleotides, L-DNA nucleotides, modified L-RNA nucleotides, modified L-DNA nucleotides and/or L-LNA nucleotides.

The detection of the nucleic acid according to the present invention can be carried out as follows:

The nucleic acid according to the present invention hybridises with one of its ends to the capture probe and with the other end to the detection probe. Afterwards unbound detection probe is removed by, e.g., one or several washing steps. The amount of bound detection probe which preferably carries a label or marker molecule, can be measured subsequently as, for example, outlined in more detail in WO/2008/052774 which is incorporated herein by reference.

As preferably used herein, the term treatment comprises in a preferred embodiment additionally or alternatively prevention and/or follow-up.

As preferably used herein, the terms disease and disorder shall be used in an interchangeable manner, if not indicated to the contrary.

As used herein, the term comprise is preferably not intended to limit the subject matter followed or described by such term. However, in an alternative embodiment the term comprises shall be understood in the meaning of containing and thus as limiting the subject matter followed or described by such term.

The various SEQ.ID. Nos., the chemical nature of the nucleic acid molecules according to the present invention and the target molecules C5a as used herein, the actual sequence thereof and the internal reference number is summarized in the following table.

| | | | |
|---|---|---|---|
| Seq.- | RNA/ | | Internal |
| ID | Peptide | Sequence | Reference |

| | | | |
|---|---|---|---|
| 1 | L-protein | TLQKKIEEIAAKYKHSVVKKCCYDGACVNNDETCEQRAARISLGPRCIKAFTECCVVASQ | human C5a |
| | | LRANISHKDMQLGR | |
| 2 | D-protein | TLQKKIEEIAAKYKHSVVKKCCYDGAAVNNDETCEQRAARISLGPRCIKAFTECCVVASQ | biotinylated |
| | | LRAKISHKDMQLGR | human D-C5a |
| | | \| | |
| | | Biotin | |
| 3 | L-RNA | 5′-AGCGUGCUUGUCCGAUUGGCGGCACCCUUGCGGGACUGGGGAGUACGCU | 172-D7-000 |
| 4 | L-RNA | 5′-CGUGCUUGUCCGAUUGGCGGCACCCUUGCGGGACUGGGGAGUACG | 172-D7-001 |
| 5 | L-RNA | 5′-GUGCUUGUCCGAUUGGCGGCACCCUUGCGGGACUGGGGAGUAC | 172-D7-002 |
| 6 | L-RNA | 5′-AGCGUGCUCGUCCGAUUGGCGGCACCCUUGCGGGACUGGGGAGUACGCU | 172-D7-003 |
| 7 | L-RNA | 5′-AGCGUGCUUGUCCGA-Spacer-GCGGCACCCUUGCGGGACUGGGGAGUACGCU | 172-D7-004 |
| 8 | L-RNA | 5′-AGCGUGCUUGUCCGAUUGGCGGCACCCU-Spacer-CGGGACUGGGGAGUACGCU | 172-D7-005 |
| 9 | L-RNA | 5′-CGUGCUUGUCCGAUUGGCGGCACCCU-Spacer-CGGGACUGGGGAGUACG | 172-D7-008 |
| 10 | L-RNA | 5′-CGUGCUUGUCCGAUUGGCGGCACCC-Spacer-GGGACUGGGGAGUACG | 172-D7-009 |
| 11 | L-RNA | 5′-CGCGCUUGUCCGAUUGGCGGCACCCUUGCGGGACUGGGGAGUGCG | 172-D7-010 |
| 12 | L-RNA | 5′-CGCGCUUGUCCGAUUGGCGGCACCCUUGCGGGACUGGGGAGCGCG | 172-D7-011 |
| 13 | L-RNA | 5′-GCGCUUGUCCGAUUGGCGGCACCCUUGCGGGACUGGGGAGCGC | 172-D7-012 |
| 14 | L-RNA | 5′-GCGCUUGUCCGAUUGGCGGCACCCU-Spacer-CGGGACUGGGGAGCGC | 172-D7-013 |
| 15 | L-RNA | 5′-GCGCUUGUCCGAUUGGCGGCACCC-Spacer-GGGACUGGGGAGCGC | 172-D7-014 |
| 16 | L-RNA | 5′-GCGCUUGUCCG-Spacer-UGGCGGCACCC-Spacer-GGGACUGGGGAGCGC | 172-D7-015 |
| 17 | L-RNA | 5′-GCGCUUGUCCGAUU-Spacer-CGGCACCC-Spacer-GGGACUGGGGAGCGC | 172-D7-016 |
| 18 | L-RNA | 5′-GCGCUGUCCGAUUGGCGGCACCC-Spacer-GGGACUGGGGGCGC | 172-D7-017 |
| 19 | L-RNA | 5′-GCGCUUGUCCGAUUGGCGGCACC-Spacer-GGACUGGGGAGCGC | 172-D7-018 |
| 20 | L-RNA | 5′-GUCCGAUUGGCGGCACCCUUGCGGGACUGGG | Type A |
| | | | Formula-1 |
| 21 | L-RNA | 5′-GUGCUGAACACGCCGCGUAGGACUUCAAUGGAGUAGAAUGGGCAGCAC | 179-A3 |
| 22 | L-RNA | 5′-GUGCUGCAACACGCCGAAUAGGUCCCGCGCGGAAGAAUGGGGCAGCAC | 179-C1 |
| 23 | L-RNA | 5′-GUGCCGCCAGACGCCGAACAGGUCGCAUCGCGAAGAAUCGGGCAGCAC | 179-D3 |
| 24 | L-RNA | 5′-GUGCUGCCAGACGCCGAACAGGUCGCAUCGCGAAGAAUCGGGUAGCAC | 179-E1 |
| 25 | L-RNA | 5′-GUGCUGCAAGACGCCGAACAGGUCCAGGAAGGGAAGAAUCGGGCAGCAC | 179-A4 |
| 26 | L-RNA | 5′-GUGCUGUCAGACGCCGAACAGGUCGCAUUGCGAAGAAUCGGGCAGCAC | 182-E6 |
| 27 | L-RNA | 5′-GUGCUGCUAAGACGCCGGAUAGGUCCUUUUAGGAAGAAUCGGAGCAC | 179-G1 |
| 28 | L-RNA | 5′-GUGCUGCAAGACGCCGAAUAGGACCGAAGUGUAGAAUCGUGCAGCAC | 182-D5 |
| 29 | L-RNA | 5′-GUGCUGAGACGCCGAACAGGACCAGCGAAAAUGGUAGAAUCGCAGCAC | 179-F2 |
| 30 | L-RNA | 5′-ASACGCCGVRYAGGWC | Type B |
| | | | Formula-1 |
| 31 | L-RNA | 5′-ASACGCCGMRYAGGWC | Type B |
| | | | Formula-2 |
| 32 | L-RNA | 5′-GWAGAAUSG | Type B |
| | | | Formula-3 |
| 33 | L-RNA | 5′-GGCUGAACACGCCGCGUAGGACUUCAAUGGAGUAGAAUGGGCAGCC | 179-A3-003 |
| 34 | L-RNA | 5′-GCUGAACACGCCGCGUAGGACUUCAAUGGAGUAGAAUGGGCAGC | 179-A3-007 |
| 35 | L-RNA | 5′-CUGAACACGCCGCGUAGGACUUCAAUGGAGUAGAAUGGGCAG | 179-A3-008 |
| 36 | L-RNA | 5′-GGCUGAACACGCCGCGUAGGACCCAAUGGGUAGAAUGGGCAGCC | 179-A3-014 |
| 37 | L-RNA | 5′-GGCUGAACACGCCGCGUAGGACCC-Spacer-GGGUAGAAUGGGCAGCC | 179-A3-042 |
| 38 | L-RNA | 5′-GCUGAACACGCCGCGUAGGACCCAAUGGGUAGAAUGGGCAGC | 179-A3-015 |
| 39 | L-RNA | 5′-GCGGAACACGCCGCGUAGGACCCAAUGGGUAGAAUGGGCCGC | 179-A3-020 |
| 40 | L-RNA | 5′-GCUGCACACGCCGCGUAGGACCCAAUGGGUAGAAUGGGCAGC | 179-A3-021 |
| 41 | L-protein | MLKKKIEEEAAKYRNAWVKKCCYDGAHRNDDETCEERAARIAIGPECIKAFKSCCAIASQ | bovine C5a |
| | | FRADEHHKNMQLGR | |
| 42 | L-protein | MLQKKIEEEAAKYKYAMLKKCCYDGAYRNDDETCEERAARIKIGPKCVKAFKDCCYIANQ | porcine C5a |
| | | VRAEQSHKNIQLGR | |
| 43 | L-RNA | 5′-GGCUAACACGCCGCGUAGGACCCAAUGGGUAGAAUGGGAGCC | 179-A3-024 |
| 44 | L-RNA | 5′-GGCCAACACGCCGCGUAGGACCCAAUGGGUAGAAUGGGGGCC | 179-A3-026 |
| 45 | L-RNA | 5′-GCCCAACACGCCGCGUAGGACCCAAUGGGUAGAAUGGGGGGC | 179-A3-029 |
| 46 | L-RNA | 5′-CGCCAACACGCCGCGUAGGACCCAAUGGGUAGAAUGGGGGCG | 179-A3-030 |
| 47 | L-RNA | 5′-CCGGAACACGCCGCGUAGGACCCAAUGGGUAGAAUGGGCCGG | 179-A3-034 |
| 48 | L-RNA | 5′-CGGGAACACGCCGCGUAGGACCCAAUGGGUAGAAUGGGCCCG | 179-A3-037 |
| 49 | L-RNA | 5′-GCUGGGCGUGUUUACUUGCUUAAUAGGGGGCCCAGC | 185-H3-001 |
| 50 | L-RNA | 5′-GCUGGGCGUGUUUACUUGCUUAAUAGGGGUCCCAGC | 185-D3 |
| 51 | L-RNA | 5′-GCUGGGCGUGUUUACUUGCUUAAUAGGGGGCCUAGC | 185-B3 |
| 52 | L-RNA | 5′-GCUGGGCGUGUUUAUUUGCUUAAUAGGGGGUCCAGC | 185-B1 |
| 53 | L-RNA | 5′-GCUGGGCGUGUUUACUUGCUUAAUAGGGAGCCCAGC | 185-F4 |
| 54 | L-RNA | 5′-GCUGGGCGUGUUUACUCGCUUAAUAGGGGACCCAGC | 185-A3 |
| 55 | L-RNA | 5′-GCUGGGGAGUGUUUACUUGCUUAAUAGGGGUCCCCAGC | 185-B4 |
| 56 | L-RNA | 5′-GCUGGGGAGUGUUUACUUGCUUAAUAGGGGUCCUCAGC | 185-G4 |
| 57 | L-RNA | 5′-GCUGGGGAGUGUUUACUUGCUUAAUAGGGAUCCUUAGC | 185-H4 |
| 58 | L-RNA | 5′-GCUGAGGAGUGUUUACUUGCUUAAUAGGGGUCCCCAGC | 185-C3 |
| 59 | L-RNA | 5′-GUGUUUAYUYGCUUAAUAGGGR | Type C |
| | | | Formula-1 |
| 60 | L-RNA | 5′-GUGUUUACUUGCUUAAUAGGGG | Type C |
| | | | Formula-2 |
| 61 | L-RNA | 5′-CGUGGCGUGUUUACUUGCUUAAUAGGGGGCCACG | 185-H3-005 |
| 62 | L-RNA | 5′-CCGCGCGUGUUUACUUGCUUAAUAGGGGGCGCGG | 185-H3-006 |
| 63 | L-RNA | 5′-UGGGCGUGUUUACUUGCUUAAUAGGGGGCCCA | 185-H3-002 |
| 64 | L-RNA | 5′-CGGGCGUGUUUACUUGCUUAAUAGGGGGCCCG | 185-H3-007 |
| 65 | L-RNA | 5′-GGGGCGUGUUUACUUGCUUAAUAGGGGGCCCC | 185-H3-014 |
| 66 | L-RNA | 5′-GGGGAGUGUUUACUUGCUUAAUAGGGGUCCCC | 185-B4-002 |
| 67 | L-RNA | 5′-GGGCGUGUUUACUUGCUUAAUAGGGGGCCC | 185-H3-003 |
| 68 | L-RNA | 5′-GGGAGUGUUUACUUGCUUAAUAGGGGUCCC | 185-B4-003 |
| 69 | L-RNA | 5′-GUACUGCGUUCGGACGUGGCAUGUUCCUUGACAAACGGUUGGCAGUAC | 182-E5 |
| 70 | L-RNA | 5′-GUGCUGCGUUCGGACGUGGCAUGUUCCUUGACAAACGGUUGGCAGCAC | 182-C5 |
| 71 | L-RNA | 5′-GUGCUGGGUUCGGACGUGGCAUGUUCCUUGAUAAACGGUUGCCAGCAC | 182-A8 |
| 72 | L-RNA | 5′-GUUCGGACGUGGCAUGUUCCUUGAYAAACGGUUG | Type D |
| | | | Formula-1 |
| 73 | L-RNA | 5′-GUGUUGCGUAGAAUGGACAUAGAGGACACGCCGCGCAGGACGCAGCAC | 179-B3 |
| 74 | L-RNA | 5′-GUGCUGCGAAGAAUGGACAAAUCGUACACGCCGAGCAGGUCGCAGUAC | 179-A2 |
| 75 | L-RNA | 5′-GUGCUGGACAGGACCAAGGUAAGGGCGGACCGAAAAACCUAGCAGCAC | 182-A5 |
| 76 | L-RNA | 5′-AGCGUGAACACGCCGAAUAGGUCCUAUAGGUGGGAAGAAUGGGCACGCU | 172-C5-000 |
| 77 | L-RNA | 5′-CCUGUGCGAAGAAUGGGCCCUAGGGAACACGCCGAAAAGGUUGCACAGG | 173-A11-000 |
| 78 | L-RNA | 5′-CCUGUGCGAAGCGCUCGGCGCAUACCGAUCAGGUCCGGCAAGCACAGG | 173-B12-000 |
| 79 | L-RNA | 5′-CGUGCAACACGGCGAAUAGCGUCCUACAGUUAGGCAGAAUGGGGCACG | 171-B1-000 |
| 80 | D-RNA | 5′-AGCGUGCUUGUCCGAUUGGCGGCACCCUUGCGGGACUGGGGAGUACGCU | 172-D7-000 |
| 81 | D-RNA | 5′-CGUGCUUGUCCGAUUGGCGGCACCCUUGCGGGACUGGGGAGUACG | 172-D7-001 |
| 82 | D-RNA | 5′-GUGCUUGUCCGAUUGGCGGCACCCUUGCGGGACUGGGGAGUAC | 172-D7-002 |
| 83 | D-RNA | 5′-AGCGUGCUCGUCCGAUUGGCGGCACCCUUGCGGGACUGGGGAGUACGCU | 172-D7-003 |
| 84 | D-RNA | 5′-AGCGUGCUUGUCCGA-Spacer-GCGGCACCCUUGCGGGACUGGGGAGUACGCU | 172-D7-004 |
| 85 | D-RNA | 5′-AGCGUGCUUGUCCGAUUGGCGGCACCCU-Spacer-CGGGACUGGGGAGUACGCU | 172-D7-005 |
| 86 | D-RNA | 5′-CGUGCUUGUCCGAUUGGCGGCACCCU-Spacer-CGGGACUGGGGAGUACG | 172-D7-008 |
| 87 | D-RNA | 5′-CGUGCUUGUCCGAUUGGCGGCACCC-Spacer-GGGACUGGGGAGUACG | 172-D7-009 |
| 88 | D-RNA | 5′-CGCGCUUGUCCGAUUGGCGGCACCCUUGCGGGACUGGGGAGUGCG | 172-D7-010 |
| 89 | D-RNA | 5′-CGCGCUUGUCCGAUUGGCGGCACCCUUGCGGGACUGGGGAGCGCG | 172-D7-011 |
| 90 | D-RNA | 5′-GCGCUUGUCCGAUUGGCGGCACCCUUGCGGGACUGGGGAGCGC | 172-D7-012 |
| 91 | D-RNA | 5′-GCGCUUGUCCGAUUGGCGGCACCCU-Spacer-CGGGACUGGGGAGCGC | 172-D7-013 |
| 92 | D-RNA | 5′-GCGCUUGUCCGAUUGGCGGCACCC-Spacer-GGGACUGGGGAGCGC | 172-D7-014 |
| 93 | D-RNA | 5′-GCGCUUGUCCG-Spacer-UGGCGGCACCC-Spacer-GGGACUGGGGAGCGC | 172-D7-015 |
| 94 | D-RNA | 5′-GCGCUUGUCCGAUU-Spacer-CGGCACCC-Spacer-GGGACUGGGGAGCGC | 172-D7-016 |
| 95 | D-RNA | 5′-GCGCUGUCCGAUUGGCGGCACCC-Spacer-GGGACUGGGGGCGC | 172-D7-017 |
| 96 | D-RNA | 5′-GCGCUUGUCCGAUUGGCGGCACC-Spacer-GGACUGGGGAGCGC | 172-D7-018 |
| 97 | D-RNA | 5′-GUCCGAUUGGCGGCACCCUUGCGGGACUGGG | Type A |
| | | | Formula-1 |
| 98 | D-RNA | 5′-GUGCUGAACACGCCGCGUAGGACUUCAAUGGAGUAGAAUGGGCAGCAC | 179-A3 |
| 99 | D-RNA | 5′-GUGCUGCAACACGCCGAAUAGGUCCCGCGCGGAAGAAUGGGGCAGCAC | 179-C1 |
| 100 | D-RNA | 5′-GUGCCGCCAGACGCCGAACAGGUCGCAUCGCGAAGAAUCGGGCAGCAC | 179-D3 |
| 101 | D-RNA | 5′-GUGCUGCCAGACGCCGAACAGGUCGCAUCGCGAAGAAUCGGGUAGCAC | 179-E1 |
| 102 | D-RNA | 5′-GUGCUGCAAGACGCCGAACAGGUCCAGGAAGGGAAGAAUCGGGCAGCAC | 179-A4 |
| 103 | D-RNA | 5′-GUGCUGUCAGACGCCGAACAGGUCGCAUUGCGAAGAAUCGGGCAGCAC | 182-E6 |
| 104 | D-RNA | 5′-GUGCUGCUAAGACGCCGGAUAGGUCCUUUUAGGAAGAAUCGGAGCAC | 179-G1 |
| 105 | D-RNA | 5′-GUGCUGCAAGACGCCGAAUAGGACCGAAGUGUAGAAUCGUGCAGCAC | 182-D5 |
| 106 | D-RNA | 5′-GUGCUGAGACGCCGAACAGGACCAGCGAAAAUGGUAGAAUCGCAGCAC | 179-F2 |
| 107 | D-RNA | 5′-ASACGCCGVRYAGGWC | Type B |
| | | | Formula-1 |
| 108 | D-RNA | 5′-ASACGCCGMRYAGGWC | Type B |
| | | | Formula-2 |
| 109 | D-RNA | 5′-GWAGAAUSG | Type B |
| | | | Formula-3 |
| 110 | D-RNA | 5′-GGCUGAACACGCCGCGUAGGACUUCAAUGGAGUAGAAUGGGCAGCC | 179-A3-003 |
| 111 | D-RNA | 5′-GCUGAACACGCCGCGUAGGACUUCAAUGGAGUAGAAUGGGCAGC | 179-A3-007 |
| 112 | D-RNA | 5′-CUGAACACGCCGCGUAGGACUUCAAUGGAGUAGAAUGGGCAG | 179-A3-008 |
| 113 | D-RNA | 5′-G-GCUGAACACGCCGCGUAGGACCCAAUGGGUAGAAUGGGCAGC-C | 179-A3-014 |
| 114 | D-RNA | 5′-G-GCUGAACACGCCGCGUAGGAC-CCSpacer-GGGUAGAAUGGGCAGC-C | 179-A3-042 |
| 115 | D-RNA | 5′-GCUGAACACGCCGCGUAGGACCCAAUGGGUAGAAUGGGCAGC | 179-A3-015 |
| 116 | D-RNA | 5′-GCGGAACACGCCGCGUAGGACCCAAUGGGUAGAAUGGGCCGC | 179-A3-020 |
| 117 | D-RNA | 5′-GCUGCACACGCCGCGUAGGACCCAAUGGGUAGAAUGGGCAGC | 179-A3-021 |
| 118 | L-protein | LLHQKVEEQAAKYKHRVPKKCCYDGARENKYETCEQRVARVTIGPHCIRAFNECCTIADK | rat C5a |
| | | IRKESHHKGMLLGR | |
| 119 | L-protein | LLRQKIEEQAAKYKHSVPKKCCYDGARVNFYETCEERVARVTIGPLCIRAFNECCTIANK | mouse C5a |
| | | IRKESPHKPVQLGR | |
| 120 | D-RNA | 5′-GGCUAACACGCCGCGUAGGACCCAAUGGGUAGAAUGGGAGCC | 179-A3-024 |
| 121 | D-RNA | 5′-GGCCAACACGCCGCGUAGGACCCAAUGGGUAGAAUGGGGGCC | 179-A3-026 |
| 122 | D-RNA | 5′-GCCCAACACGCCGCGUAGGACCCAAUGGGUAGAAUGGGGGGC | 179-A3-029 |
| 123 | D-RNA | 5′-CGCCAACACGCCGCGUAGGACCCAAUGGGUAGAAUGGGGGCG | 179-A3-030 |
| 124 | D-RNA | 5′-CCGGAACACGCCGCGUAGGACCCAAUGGGUAGAAUGGGCCGG | 179-A3-034 |
| 125 | D-RNA | 5′-CGGGAACACGCCGCGUAGGACCCAAUGGGUAGAAUGGGCCCG | 179-A3-037 |
| 126 | D-RNA | 5′-GCUGGGCGUGUUUACUUGCUUAAUAGGGGGCCCAGC | 185-H3-001 |
| 127 | D-RNA | 5′-GCUGGGCGUGUUUACUUGCUUAAUAGGGGUCCCAGC | 185-D3 |
| 128 | D-RNA | 5′-GCUGGGCGUGUUUACUUGCUUAAUAGGGGGCCUAGC | 185-B3 |
| 129 | D-RNA | 5′-GCUGGGCGUGUUUAUUUGCUUAAUAGGGGGUCCAGC | 185-B1 |
| 130 | D-RNA | 5′-GCUGGGCGUGUUUACUUGCUUAAUAGGGAGCCCAGC | 185-F4 |
| 131 | D-RNA | 5′-GCUGGGCGUGUUUACUCGCUUAAUAGGGGACCCAGC | 185-A3 |
| 132 | D-RNA | 5′-GCUGGGGAGUGUUUACUUGCUUAAUAGGGGUCCCCAGC | 185-B4 |
| 133 | D-RNA | 5′-GCUGGGGAGUGUUUACUUGCUUAAUAGGGGUCCUCAGC | 185-G4 |
| 134 | D-RNA | 5′-GCUGGGGAGUGUUUACUUGCUUAAUAGGGAUCCUUAGC | 185-H4 |
| 135 | D-RNA | 5′-GCUGAGGAGUGUUUACUUGCUUAAUAGGGGUCCCCAGC | 185-C3 |
| 136 | D-RNA | 5′-GUGUUUAYUYGCUUAAUAGGGR | Type C |
| | | | Formula-1 |
| 137 | D-RNA | 5′-GUGUUUACUUGCUUAAUAGGGG | Type C |
| | | | Formula-2 |
| 138 | D-RNA | 5′-CGUGGCGUGUUUACUUGCUUAAUAGGGGGCCACG | 185-H3-005 |
| 139 | D-RNA | 5′-CCGCGCGUGUUUACUUGCUUAAUAGGGGGCGCGG | 185-H3-006 |
| 140 | D-RNA | 5′-UGGGCGUGUUUACUUGCUUAAUAGGGGGCCCA | 185-H3-002 |
| 141 | D-RNA | 5′-CGGGCGUGUUUACUUGCUUAAUAGGGGGCCCG | 185-H3-007 |
| 142 | D-RNA | 5′-GGGGCGUGUUUACUUGCUUAAUAGGGGGCCCC | 185-H3-014 |
| 143 | D-RNA | 5′-GGGGAGUGUUUACUUGCUUAAUAGGGGUCCCC | 185-B4-002 |
| 144 | D-RNA | 5′-GGGCGUGUUUACUUGCUUAAUAGGGGGCCC | 185-H3-003 |
| 145 | D-RNA | 5′-GGGAGUGUUUACUUGCUUAAUAGGGGUCCC | 185-B4-003 |
| 146 | D-RNA | 5′-GUACUGCGUUCGGACGUGGCAUGUUCCUUGACAAACGGUUGGCAGUAC | 182-E5 |
| 147 | D-RNA | 5′-GUGCUGCGUUCGGACGUGGCAUGUUCCUUGACAAACGGUUGGCAGCAC | 182-C5 |
| 148 | D-RNA | 5′-GUGCUGGGUUCGGACGUGGCAUGUUCCUUGAUAAACGGUUGCCAGCAC | 182-A8 |
| 149 | D-RNA | 5′-GUUCGGACGUGGCAUGUUCCUUGAYAAACGGUUG | Type D |
| | | | Formula-1 |
| 150 | D-RNA | 5′-GUGUUGCGUAGAAUGGACAUAGAGGACACGCCGCGCAGGACGCAGCAC | 179-B3 |
| 151 | D-RNA | 5′-GUGCUGCGAAGAAUGGACAAAUCGUACACGCCGAGCAGGUCGCAGUAC | 179-A2 |
| 152 | D-RNA | 5′-GUGCUGGACAGGACCAAGGUAAGGGCGGACCGAAAAACCUAGCAGCAC | 182-A5 |
| 153 | D-RNA | 5′-AGCGUGAACACGCCGAAUAGGUCCUAUAGGUGGGAAGAAUGGGCACGCU | 172-C5-000 |
| 154 | D-RNA | 5′-CCUGUGCGAAGAAUGGGCCCUAGGGAACACGCCGAAAAGGUUGCACAGG | 173-A11-000 |
| 155 | D-RNA | 5′-CCUGUGCGAAGCGCUCGGCGCAUACCGAUCAGGUCCGGCAAGCACAGG | 173-B12-000 |
| 156 | D-RNA | 5′-CGUGCAACACGGCGAAUAGCGUCCUACAGUUAGGCAGAAUGGGGCACG | 171-B1-000 |
| 157 | L-RNA/D- | 5′-ggAGCGUGCUUGUCCGAUUGGCGGCACCCUUGCGGGACUGGGGAGUACGCU | 172-D7-000 |
| | RNA (gg) | | |
| 158 | L-RNA/D- | 5′-ggGCGCUUGUCCGAUUGGCGGCACCCU-Spacer-CGGGACUGGGGAGCGC | 172-D7-013 |
| | RNA (gg) | | |
| 159 | L-RNA/D- | 5′-ggGGCUGAACACGCCGCGUAGGACCCAAUGGGUAGAAUGGGCAGCC | 179-A3-014 |
| | RNA (gg) | | |
| 160 | L-RNA/D- | 5′-ggGCUGAACACGCCGCGUAGGACCCAAUGGGUAGAAUGGGCAGC | 179-A3-015 |
| | RNA (gg) | | |
| 161 | L-RNA/D- | 5′-ggGCUGGGCGUGUUUACUUGCUUAAUAGGGGGCCCAGC | 185-H3-001 |
| | RNA (gg) | | |
| 162 | L-RNA/D- | 5′-ggUGGGCGUGUUUACUUGCUUAAUAGGGGGCCCA | 185-H3-002 |
| | RNA (gg) | | |
| 163 | L-RNA/D- | 5′-ggGGGGCGUGUUUACUUGCUUAAUAGGGGGCCCC | 185-H3-014 |
| | RNA (gg) | | |
| 164 | L-RNA/D- | 5′-ggGGGCGUGUUUACUUGCUUAAUAGGGGGCCC | 185-H3-003 |
| | RNA (gg) | | |
| 165 | L-RNA/D- | 5′-ggGUACUGCGUUCGGACGUGGCAUGUUCCUUGACAAACGGUUGGCAGUAC | 182-E5 |
| | RNA (gg) | | |
| 166 | L-RNA/D- | 5′-ggGUGCUGCGUUCGGACGUGGCAUGUUCCUUGACAAACGGUUGGCAGCAC | 182-C5 |
| | RNA (gg) | | |
| 167 | L-RNA | 5′-PEG-GCGCUUGUCCGAUUGGCGGCACCCU-Spacer-CGGGACUGGGGAGCGC | 172-D7-013-5′-PEG |
| 168 | L-RNA | 5′-PEG-GGCUGAACACGCCGCGUAGGACCCAAUGGGUAGAAUGGGCAGCC | 179-A3-014-5′-PEG |
| 169 | L-RNA | 5′-PEG-GCUGGGCGUGUUUACUUGCUUAAUAGGGGGCCCAGC | 185-H3-001-5′-PEG |
| 170 | L-RNA | 5′-PEG-GGGGCGUGUUUACUUGCUUAAUAGGGGGCCCC | 185-H3-014-5′-PEG |
| 171 | L-protein | TLQKKIEEIAAKYKHSVVKKCCYDGACVNNDETCEQRAARISLGPRCIKAFTECCVVASQ | Human C5, alpha |
| | | LRANISHKDMQLGRLHMKTLLPVSKPEIRSYFPESWLWEVHLVPRRKQLQFALPDSLTTW | chain |
| | | EIQGIGISNTGICVADTVKAKVFKDVFLEMNIPYSVVRGEQIQLKGTVYNYRTSGMQFCV | |
| | | KMSAVEGICTSESPVIDHQGTKSSKCVRQKVEGSSSHLVTFTVLPLEIGLHNINFSLETW | |
| | | FGKEILVKTLRVVPEGVKRESYSGVTLDPRGIYGTISRRKEFPYRIPLDLVPKTEIKRIL | |
| | | SVKGLLVGEILSAVLSQEGINILTHLPKGSAEAELMSVVPVFYVFHYLETGNHWNIFHSD | |
| | | PLIEKQKLKKKLKEGMLSIMSYRNADYSYSVWKGGSASTWLTAFALRVLGQVNKYVEQNQ | |
| | | NSICNSLLWLVENYQLDNGSFKENSQYQPIKLQGTLPVEARENSLYLTAFTVIGIRKAFD | |
| | | ICPLVKIDTALIKADNFLLENTLPAQSTFTLAISAYALSLGDKTHPQFRSIVSALKREAL | |
| | | VKGNPPIYRFWKDNLQHKDSSVPNTGTARMVETTAYALLTSLNLKDINYVNPVIKWLSEE | |
| | | QRYGGGFYSTQDTINAIEGLTEYSLLVKQLRLSMDIDVSYKHKGALHNYKMTDKNFLGRP | |
| | | VEVLLNDDLIVSTGFGSGLATVHVTTVVHKTSTSEEVCSFYLKIDTQDIEASHYRGYGNS | |
| | | DYKRIVACASYKPSREESSSGSSHAVMDISLPTGISANEEDLKALVEGVDQLFTDYQIKD | |
| | | GHVILQLNSIPSSDFLCVRFRIFELFEVGFLSPATFTVYEYHRPDKQCTMFYSTSNIKIQ | |
| | | KVCEGAACKCVEADCGQMQEELDLTISAETRKQTACKPEIAYAYKVSITSITVENVFVKY | |
| | | KATLLDIYKTGEAVAEKDSEITFIKKVTCTNAELVKGRQYLIMGKEALQIKYNFSFRYIY | |
| | | PLDSLTWIEYWPRDTTCSSCQAFLANLDEFAEDIFLNGC | |
| 172 | L-protein | QEQTYVISAPKIFRVGASENIVIQVYGYTEAFDATISIKSYPDKKFSYSSGHVHLSSE | Human C5, beta chain |
| | | NKFQNSAILTIQPKQLPGGQNPVSYVYLEVVSKHFSKSKRMPITYDNGFLFIHTDKPV | |
| | | YTPDQSVKVRVYSLNDDLKPAKRETVLTFIDPEGSEVDMVEEIDHIGIISFPDFKIPS | |
| | | NPRYGMWTIKAKYKEDFSTTGTAYFEVKEYVLPHFSVSIEPEYNFIGYKNFKNFEITI | |
| | | KARYFYNKVVTEADVYITFGIREDLKDDQKEMMQTAMQNTMLINGIAQVTFDSETAVK | |
| | | ELSYYSLEDLNNKYLYIAVTVIESTGGFSEEAEIPGIKYVLSPYKLNLVATPLFLKPG | |
| | | IPYPIKVQVKDSLDQLVGGVPVILNAQTIDVNQETSDLDPSKSVTRVDDGVASFVLNL | |
| | | PSGVTVLEFNVKTDAPDLPEENQAREGYRAIAYSSLSQSYLYIDWTDNHKALLVGEHL | |
| | | NIIVTPKSPYIDKITHYNYLILSKGKIIHFGTREKFSDASYQSINIPVTQNMVPSSRL | |
| | | LVYYIVTGEQTAELVSDSVWLNIEEKCGNQLQVHLSPDADAYSPGQTVSLNMATGMDS | |
| | | WVALAAVDSAVYGVQRGAKKPLERVFQFLEKSDLGCGAGGGLNNANVFHLAGLTFLTN | |
| | | ANADDSQENDEPCKEIL | |
| 173 | L-RNA | 5′ X₁X₂X₃GYGCX₄Y; whereby | Type A Formula-2-5′ |
| | | X₁ is A or absent, | |
| | | X₂ is G or absent, | |
| | | X₃ is C or absent, | |
| | | X₄ is U or absent. | |
| 174 | L-RNA | 5′ GX₅GYRCX₆X₇X₈; whereby | Type A Formula-2-3′ |
| | | X₅ is A or absent, | |
| | | X₆ is G or absent, | |
| | | X₇ is C or absent, | |
| | | X₈ is U or absent. | |
| 175 | L-RNA | 5′ X₃GYGCX₄U; whereby | Type A Formula-3-5′ |
| | | X₃ is C or absent, | |
| | | X₄ is U. | |
| 176 | L-RNA | 5′ GX₅GYGCX₆; whereby | Type A Formula-3-3′ |
| | | X₅ is A, | |
| | | X₆ is G or absent. | |
| 177 | L-RNA | 5′ X₁X₂SBBX₃X₄X₅, whereby | Type B Formula-4-5′ |
| | | X₁ is G or absent, | |
| | | X₂ is U or absent, | |
| | | X₃ is B or absent, | |
| | | X₄ is Y or absent, | |
| | | X₅ is M or absent. | |
| 178 | L-RNA | 5′ X₆X₇X₈VVSX₉X_{10;} whereby | Type B Formula-4-3′ |
| | | X₆ is K or absent, | |
| | | X₇ is G or absent | |
| | | X₈ is N or absent, | |
| | | X₉ is A or absent, | |
| | | X₁₀ is C or absent. | |
| 179 | L-RNA | 5′ X₁X₂GCVX₃X₄X₅, whereby | Type B Formula-5-5′ |
| | | X₁ is G or absent, | |
| | | X₂ is U or absent, | |
| | | X₃ is G, | |
| | | X₄ is C, | |
| | | X₅ is absent. | |
| 180 | L-RNA | 5′ X₆X₇X₈AGCX₉X₁₀, whereby | Type B Formula-5-3′ |
| | | X₆ is absent, | |
| | | X₇ is G, | |
| | | X₈ is C, | |
| | | X₉ is A or absent, | |
| | | X₁₀ is C or absent. | |
| 181 | L-RNA | 5′ X₁X₂GCCX₃X₄X₅, whereby | Type B Formula-6-5′ |
| | | X₁ is G or absent, | |
| | | X₂ is U or absent, | |
| | | X₃ is G, | |
| | | X₄ is C, | |
| | | X₅ is C. | |
| 182 | L-RNA | 5′ X₁X₂X₃BVGX₄M, whereby | Type C Formula-3-5′ |
| | | X₁ is G or absent, | |
| | | X₂ is C or absent, | |
| | | X₃ is B or absent, | |
| | | X₄ is G or absent, | |
| 183 | L-RNA | 5′ NX₅YBNX₆X₇X₈, whereby | Type C Formula-3-3′ |
| | | X₅ is C or absent, | |
| | | X₆ is V or absent, | |
| | | X₇ is G or absent, | |
| | | X₈ is C or absent. | |
| 184 | D-DNA | 5′-ATGCTACAAGAGAAGATAGAAG | C5a-Primer-I |
| 185 | D-DNA | 5′-CTAGCATGCTTACCTTCCCAATTGC | C5a-Primer-II |
| 186 | L-Protein | MLQEKIEEIAAKYKHLVVKKCCYDGVRINHDETCEQRAARISVGPRCVKAFT | His6-macC5a; C5a of |
| | | ECCVVASQLRANNSHKDLQLGR | macaque mullata and |
| | | | macaque fascicularis |
| 187 | L-RNA | 5′-PEG-CCCCGGGGGAUAAUUCGUUCAUUUGUGCGGGG | 185-H3-014-REVERSE-5′-PEG |
| 188 | L-RNA | 5′-GCCGGGUGUUUACUUGCUUAAUAGGGGCCGGC | 185-H3-018 |
| 189 | L-RNA | 5′-GGGGCGUGUUUACUUGCUUAAUAGGGAGCCCC | 185-H3-019 |
| 190 | L-RNA | 5′-GGCCGAACACGCCGCGUAGGACCCAAUGGGUAGAAUGGGCGGCC | 179-A3-046 |
| 191 | L-RNA | 5′-GGCUGAACACGCCGCGUAGGACCAAUGGUAGAAUGGGCAGCC | 179-A3-047 |
| 192 | L-RNA | 5′-GGCCGAAGACGCCGCGUAGGACCCAAUGGGUAGAAUCGGCGGCC | 179-A3-050 |
| 193 | L-RNA | 5′-GGCCGAAGACGCCGAACAGGACCCAAUGGGUAGAAUCGGCGGCC | 179-A3-051 |
| 194 | L-RNA | 5′-GGCUGAACACGCCGCGUAGGACGCAAUGCGUAGAAUGGGCAGCC | 179-A3-052 |
| 195 | L-RNA | 5′-GGCUGAACACGCCGCGUAGGACGCUUUGCGUAGAAUGGGCAGCC | 179-A3-053 |
| 196 | L-RNA | 5′-GCGGCGUUCGGACGUGGCAUGUUCCUUGACAAACGGUUGGCCGC | 182-E5-007 |
| 197 | L-RNA | 5′-UACUGCGUUCGGACGUGGCAUGUUCCUUGACAAACGGUUGGCAGUA | 182-E5-009 |
| 198 | L-RNA | 5′-ACUGCGUUCGGACGUGGCAUGUUCCUUGACAAACGGUUGGCAGU | 182-E5-010 |
| 199 | L-RNA | 5′-UACUGCGUUCGGACGUGGCAUGUUCCUUGACAAACGGUUGGCAGUAC | 182-E5-011 |
| 200 | L-RNA | GCUGCGUUCGGACGUGGCAUGUUCCUUGACAAACGGUUGGCAGC | 182-E5-013 |
| 201 | L-RNA | 5′-GCUGGGUUCGGACGUGGCAUGUUCCUUGAUAAACGGUUGCCAGC | 182-E5-014 |
| 202 | L-RNA | 5′-GUACUGCGUUCGGACGUGGCAUGUUCCUUGACAAACGGUUGGCAGU | 182-E5-015 |
| 203 | L-RNA | 5′-GUGCUGGGUUCGGACGUGGCAUGUUCCUUGAUAAACGGUUGCCAGCAC | 182-E5-016 |
| 204 | L-RNA | 5′-UGCUGCAAGACGCCGAAUAGGACCGAAGUGUAGAAUCGUGCAGCA | 182-D5-002 |
| 205 | L-RNA | 5′-GCUGCAAGACGCCGAAUAGGACCGAAGUGUAGAAUCGUGCAGC | 182-D5-003 |
| 206 | L-RNA | 5′-CUGCAAGACGCCGAAUAGGACCGAAGUGUAGAAUCGUGCAG | 182-D5-004 |
| 207 | L-RNA | 5′-UGCUGGACAGGACCAAGGUAAGGGCGGACCGAAAAACCUAGCAGCA | 182-A5-002 |
| 208 | L-RNA | 5′-UGCUGUCAGACGCCGAACAGGUCGCAUUGCGAAGAAUCGGGCAGCA | 182-E6-002 |
| 209 | L-RNA | 5′-GCUGUCAGACGCCGAACAGGUCGCAUUGCGAAGAAUCGGGCAGC | 182-E6-003 |
| 210 | L-RNA | 5′-CUGUCAGACGCCGAACAGGUCGCAUUGCGAAGAAUCGGGCAG | 182-E6-004 |
| 211 | L-RNA | 5′-GUGCUGCCAGACGCCGAACAGGUCGCAUUGCGAAGAAUCGGGCAGCAC | 182-E6-005 |
| 212 | D-DNA | 5′-TGGGTGGCATTAACAGCAGTG | CynoC5a_up (Primer) |
| 213 | D-DNA | 5′-TCCCAGGTAGTTACAGAATCAGGT | CynoC5a_low (Primer) |
| 214 | L-RNA | 5′-WAGAAUSG | Type B Formula-3a |
| 215 | L-RNA | 5′-GUAGAAUGG | Type B Formula-8 |
| 216 | L-RNA | 5′-GAGAAUGG | Type B Formula-8a |
| 217 | L-RNA | 5′ X₁X₂RCKGS, whereby | Type D Formula-2 5′ |
| | | X₁ is G or absent, | |
| | | X₂ is U or absent. | |
| 218 | L-RNA | 5′ SCMGYX₃X_{4;} whereby | Type D Formula-2 3′ |
| | | X₃ is A or absent, | |
| | | X₄ is C or absent. | |
| 219 | D-RNA | fC-mG-fC-fC-fG-fC-mG-mG-fU-fC-fU-fC-mC-mG-mG-CGCU- | C5C6 (aptamer) |
| | | mG-mA-mG-fU-fC-fU-mG-mA--mG-fU-fU-fU-fA-fC-fC-fU-mG- | |
| | | mG | |
| | | m = 2′-Methyl RNA | |
| | | f = 2′Fluoro RNA | |
| 220 | L-RNA | 5′PEG-UAAGGAAACUCGGUCUGAUGCGGUAGCGCUGUGCAGAGCU | POC |
| 221 | L-RNA | 5′-PEG- | 182-E5-5′PEG |
| | | GUACUGCGUUCGGACGUGGCAUGUUCCUUGACAAACGGUUGGCAGUAC | |

The present invention is further illustrated by the figures, examples and the sequence listing from which further features, embodiments and advantages may be taken, wherein

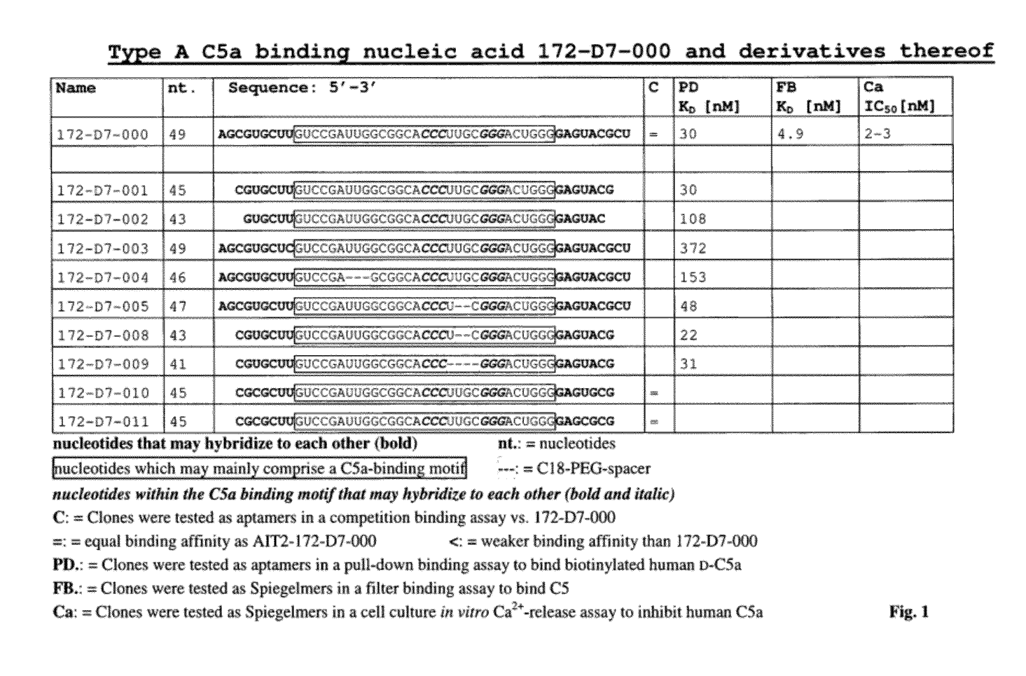

FIG. 1 shows an alignment of sequences of RNA ligand 172-D7-000 and the derivatives of RNA ligand 172-D7-000 binding to human C5a indicating the sequence motif (“Type A”) that is in a preferred embodiment in its entirety essential for binding to human C5a;

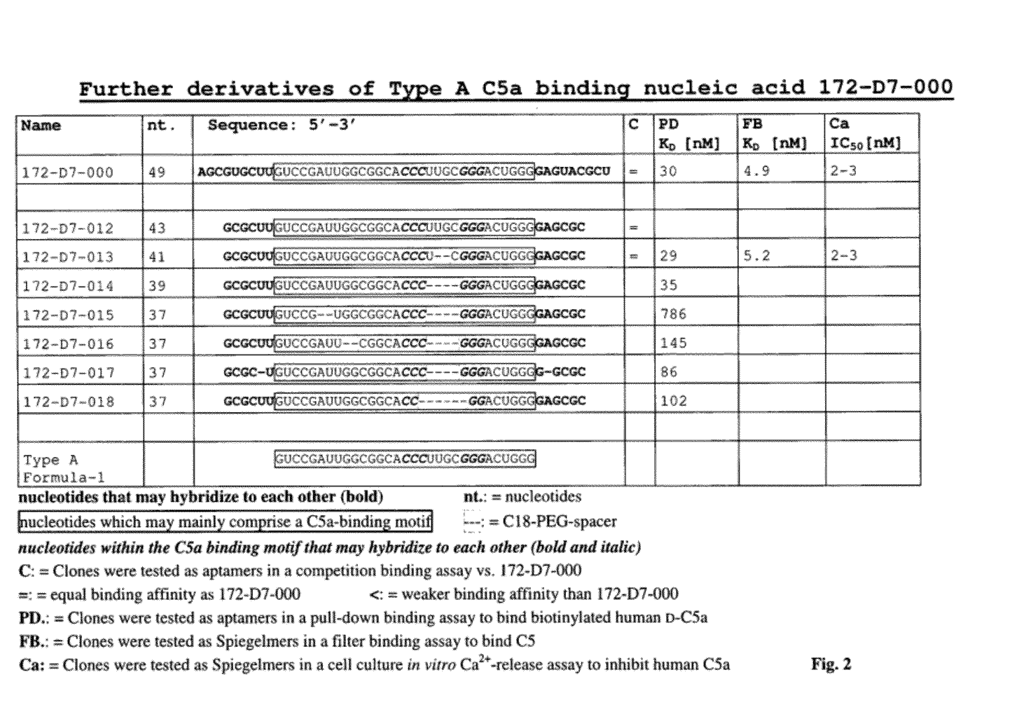

FIG. 2 shows further derivatives of RNA ligand 172-D7-000 (human C5a RNA ligand of sequence motif “Type A”);

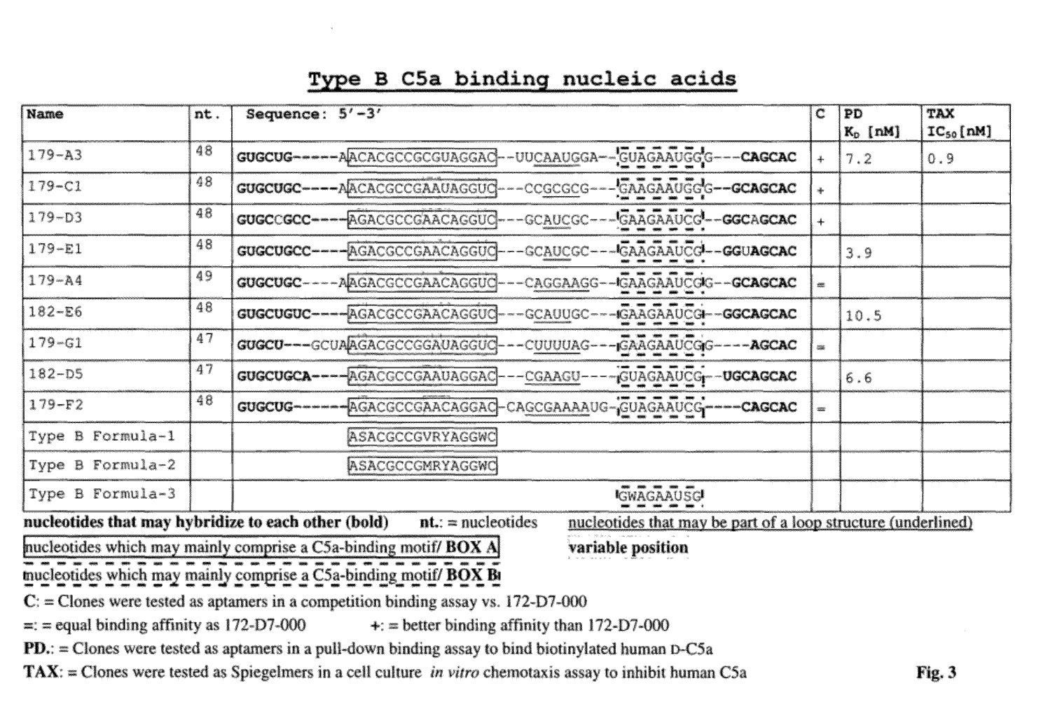

FIG. 3 shows an alignment of sequences of related RNA ligands binding to human C5a indicating the sequence motif (“Type B”) that is in a preferred embodiment in its entirety essential for binding to human C5a;

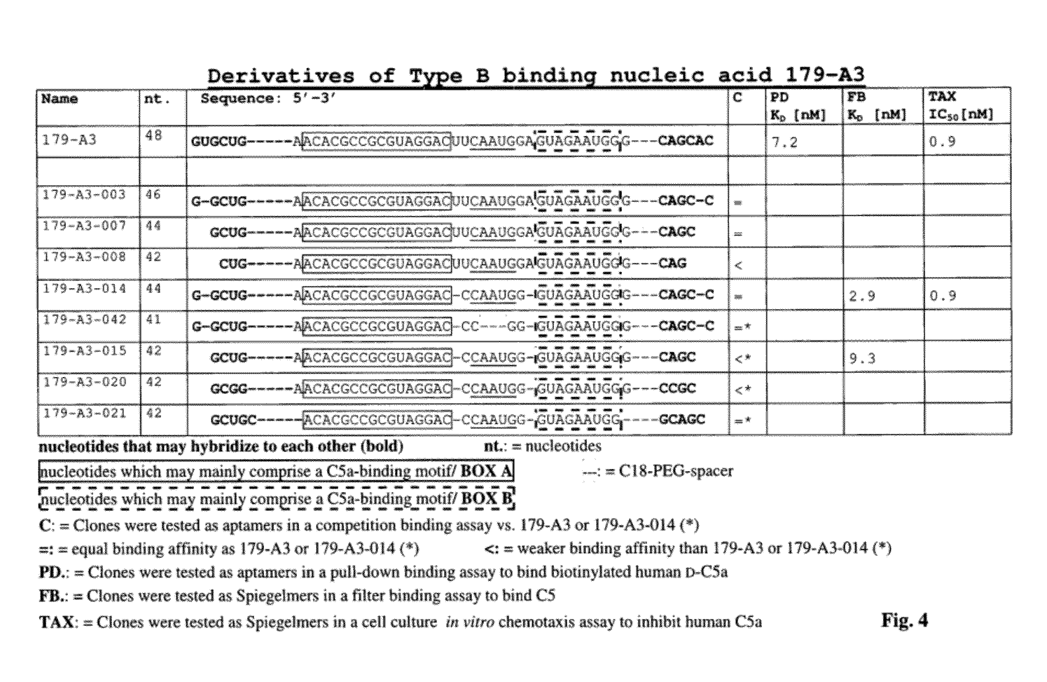

FIG. 4 shows derivatives of RNA ligands 179-A3 (human C5a RNA ligand of sequence motif “Type B”);

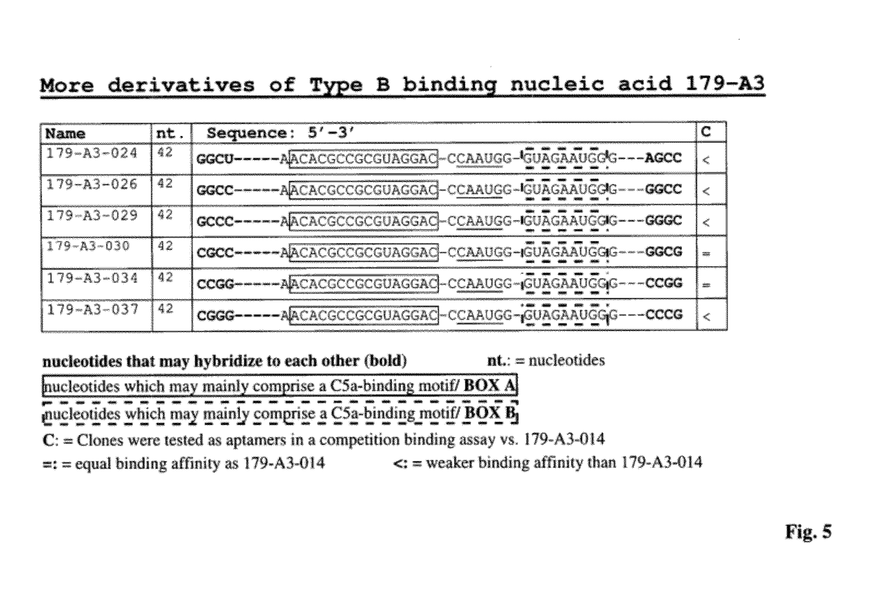

FIG. 5 shows more derivatives of RNA ligand 179-A3 (human C5a RNA ligand of sequence motif “Type B”);

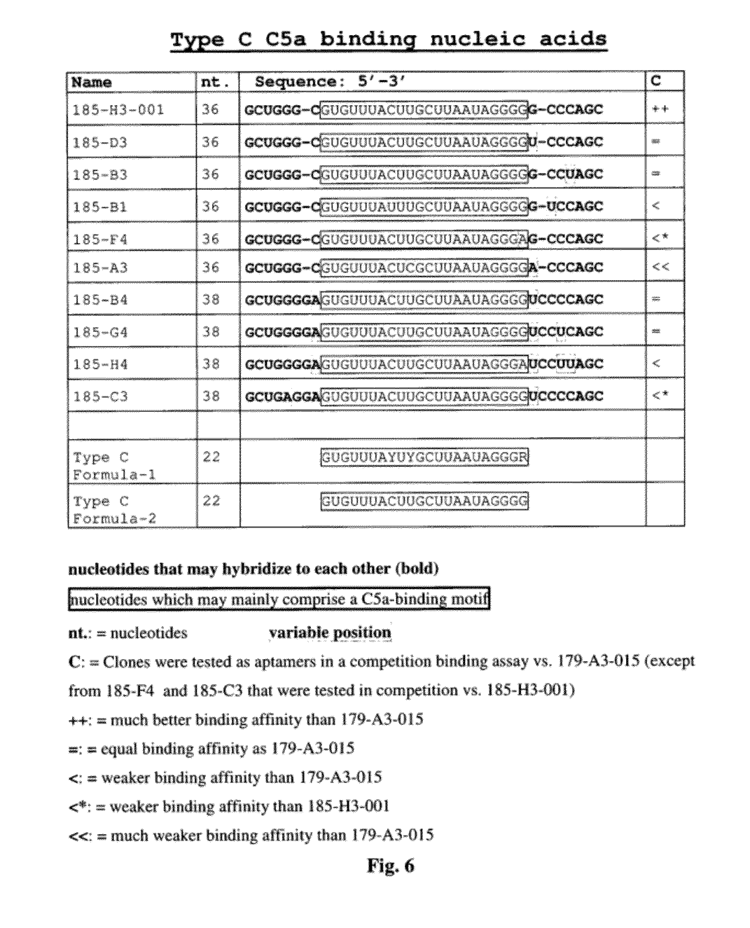

FIG. 6 shows an alignment of sequences of related RNA ligands binding to human C5a indicating the sequence motif (“Type C”) that is in a preferred embodiment in its entirety essential for binding to human C5a;

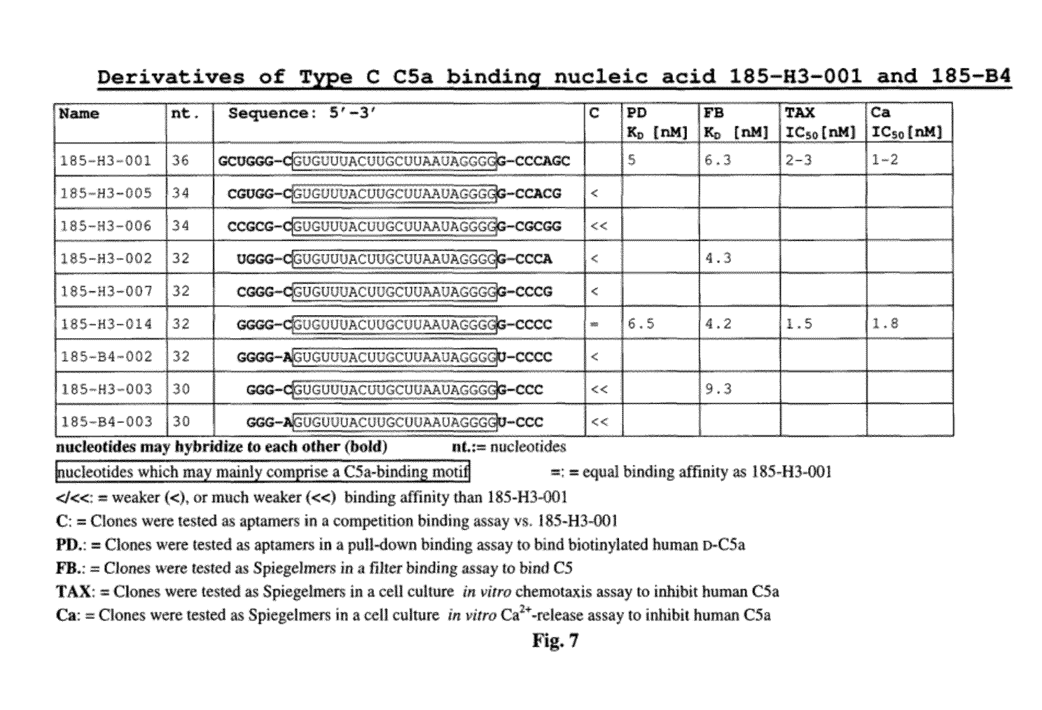

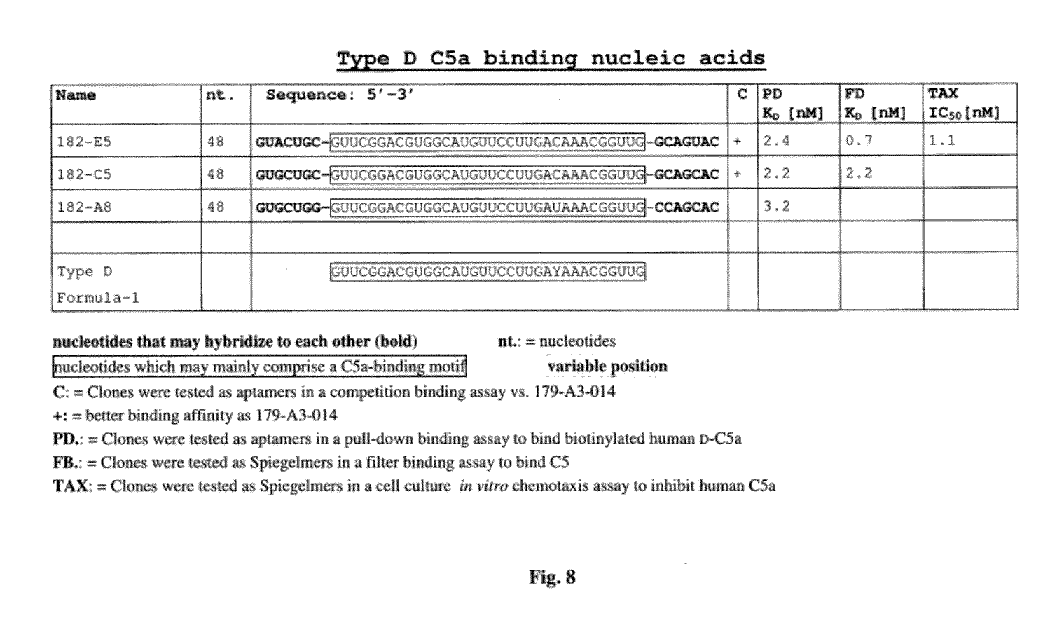

FIG. 7 shows derivatives of RNA ligands 185-H3-001 and 185-B4 (human C5a RNA ligands of sequence motif “Type C”);

FIG. 8 shows an alignment of sequences of related RNA ligands binding to human C5a indicating the sequence motif (“Type D”) that is in a preferred embodiment in its entirety essential for binding to human C5a;

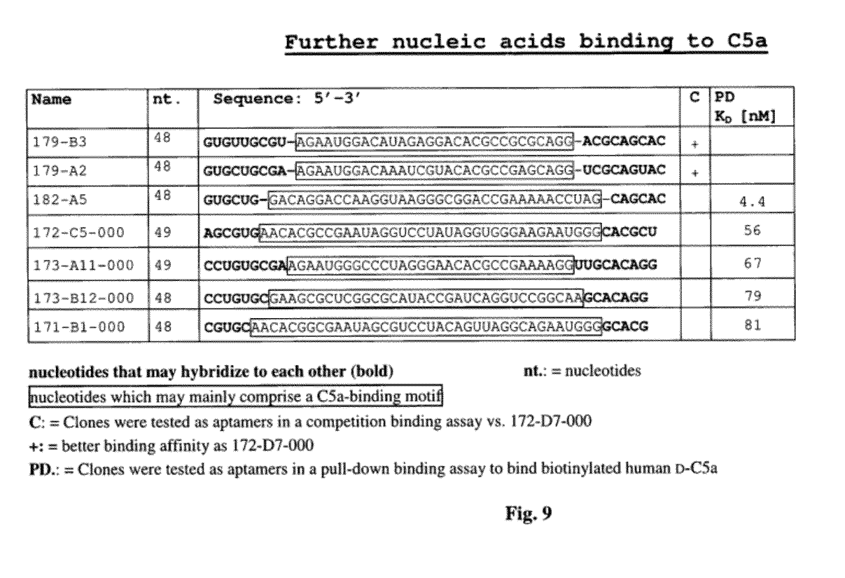

FIG. 9 shows a table of sequences of several different RNA ligands binding to human C5a which can not be related to the C5a binding sequence motifs “Type A”, “Type B”; “Type C” or “Type D”;

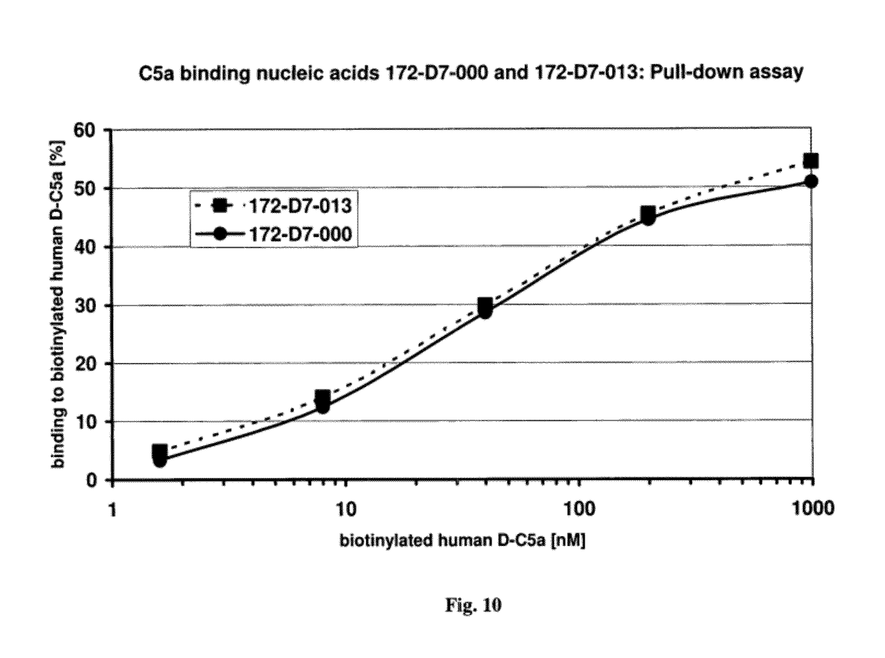

FIG. 10 shows the result of a binding analysis of the aptamers of C5a binding nucleic acids 172-D7-000 and 172-D7-013 to biotinylated human D-05a at 37° C., represented as binding of the aptamers over concentration of biotinylated human D-05a;

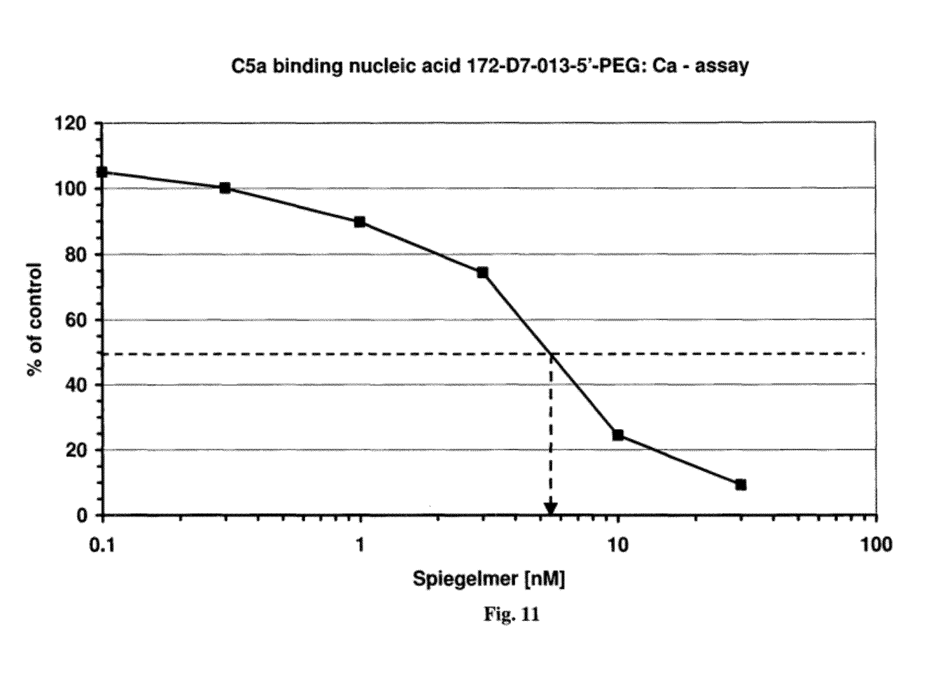

FIG. 11 shows the efficacy of Spiegelmer 172-D7-013-5′-PEG in a calcium release assay; cells were stimulated with 3 nM human C5s preincubated at 37° C. with various amounts of Spiegelmer 172-D7-013-5′-PEG, represented as percentage of control over concentration of 172-D7-013-5′-PEG;

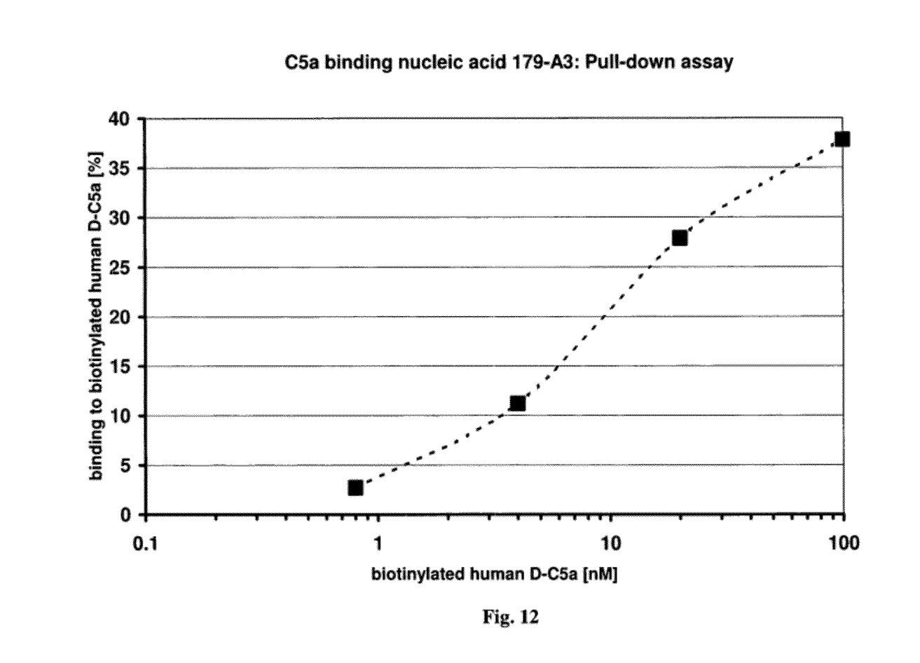

FIG. 12 shows the result of a binding analysis of the aptamer of C5a binding nucleic acid 179-A3 to biotinylated human D-05a at 37° C., represented as binding of the aptamer over concentration of biotinylated human D-05a;

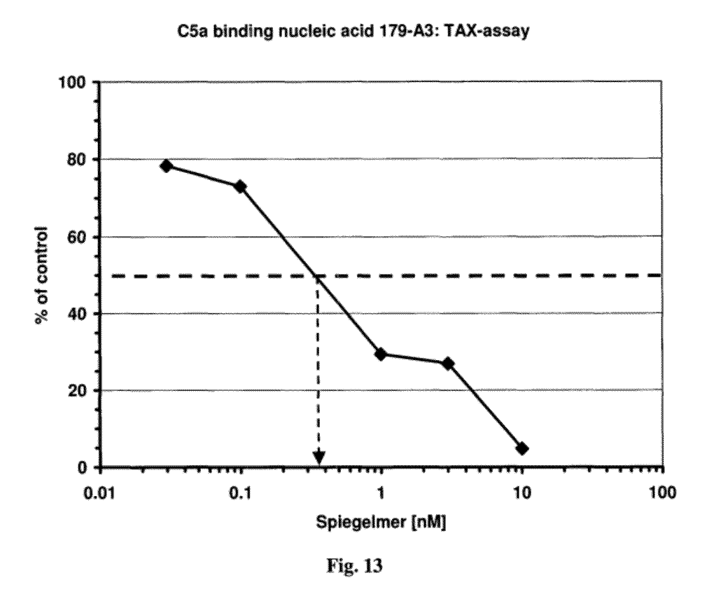

FIG. 13 shows the efficacy of Spiegelmer 179-A3 in a chemotaxis assay; cells were allowed to migrate towards 0.1 nM human C5a preincubated at 37° C. with various amounts of Spiegelmer 179-A3, represented as percentage of control over concentration of Spiegelmer 179-A3;

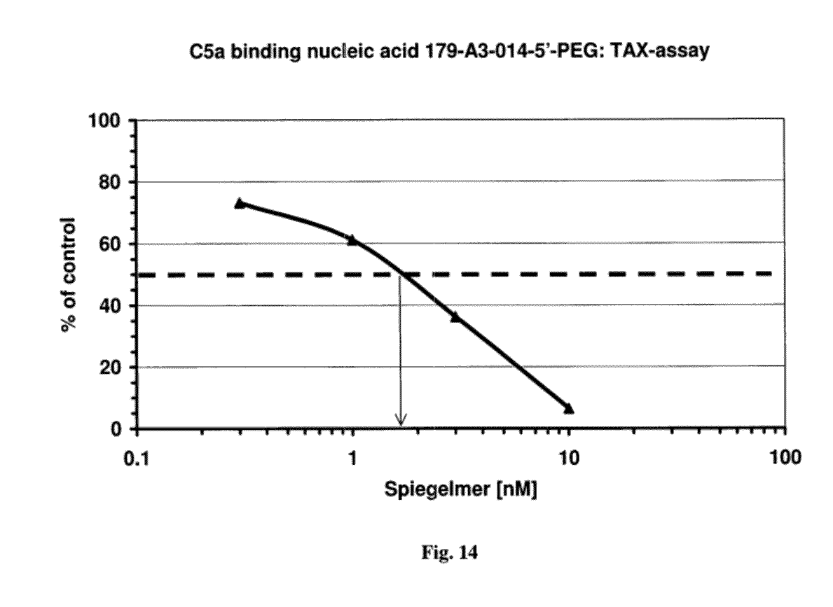

FIG. 14 shows the efficacy of Spiegelmer 179-A3-014-5′-PEG in a chemotaxis assay; cells were allowed to migrate towards 0.1 nM human C5a preincubated at 37° C. with various amounts of Spiegelmer 179-A3-014-5′-PEG, represented as percentage of control over concentration of Spiegelmer 179-A3-014-5′-PEG;

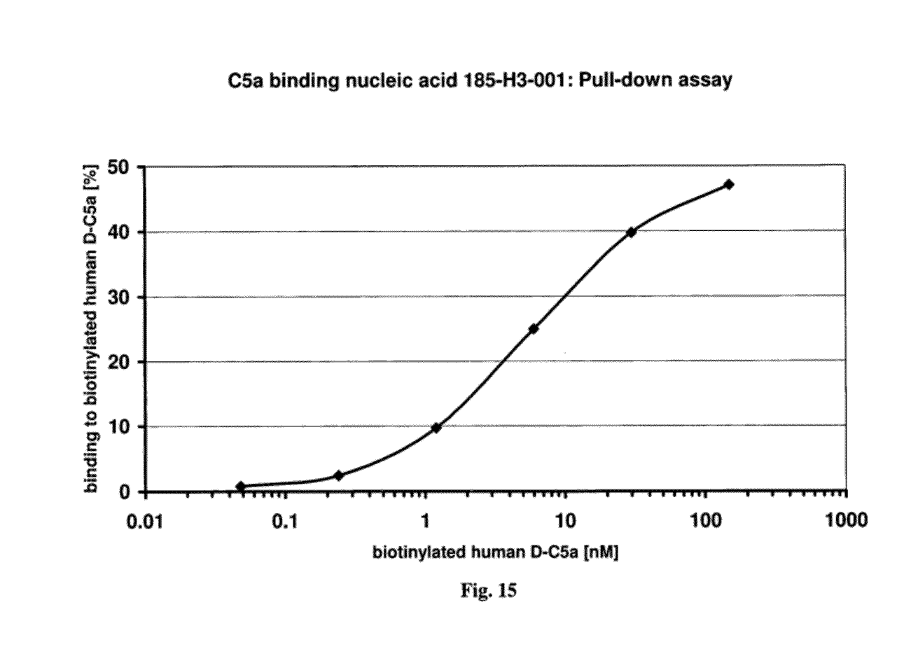

FIG. 15 shows the result of a binding analysis of the aptamer of C5a binding nucleic acid 185-H3-001 to biotinylated human D-05a at 37° C., represented as binding of the aptamer over concentration of biotinylated human D-05a;

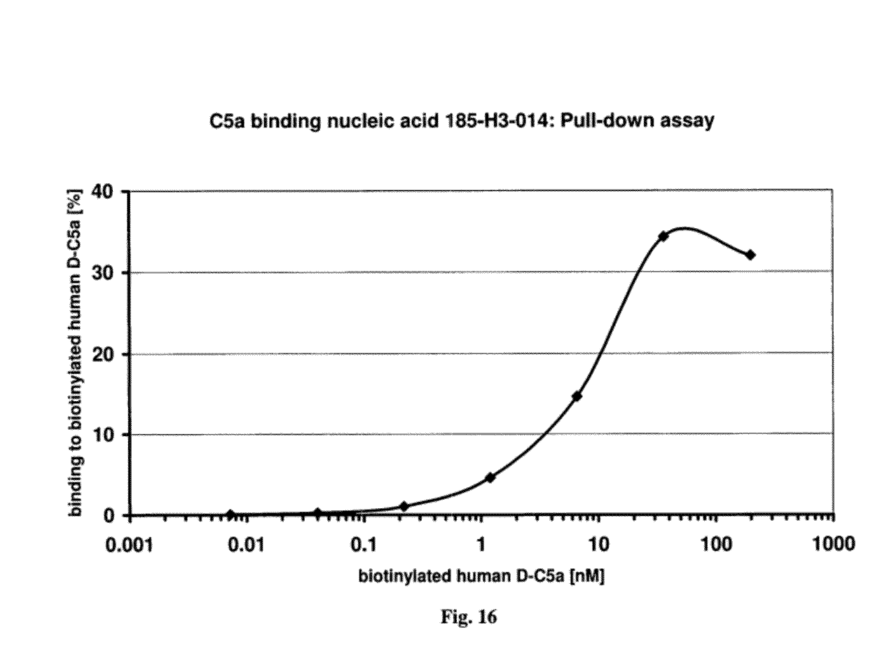

FIG. 16 shows the result of a binding analysis of the aptamer of C5a binding nucleic acid 185-H3-014 to biotinylated human D-05a at 37° C., represented as binding of the aptamer over concentration of biotinylated human D-05a;

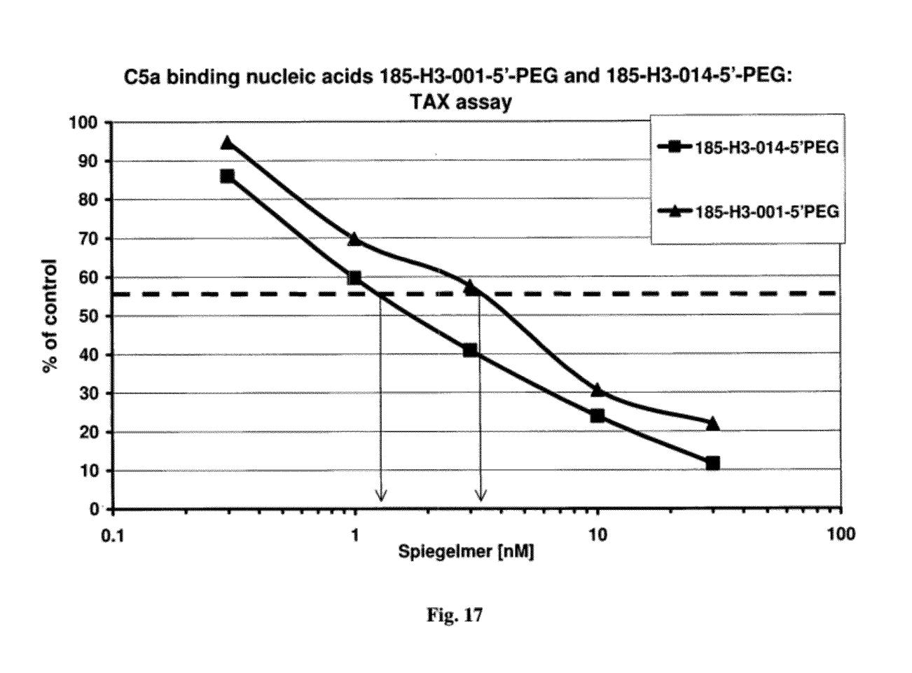

FIG. 17 shows the efficacy of Spiegelmers 185-H3-001-5′-PEG and 185-H3-014-5′-PEG in a chemotaxis assay; cells were allowed to migrate towards 0.1 nM human C5a preincubated at 37° C. with various amounts of Spiegelmers 185-H3-001-5′-PEG and 185-H3-014-5′-PEG, represented as percentage of control over concentration of Spiegelmers 185-H3-001-5′-PEG and 185-H3-014-5′-PEG;

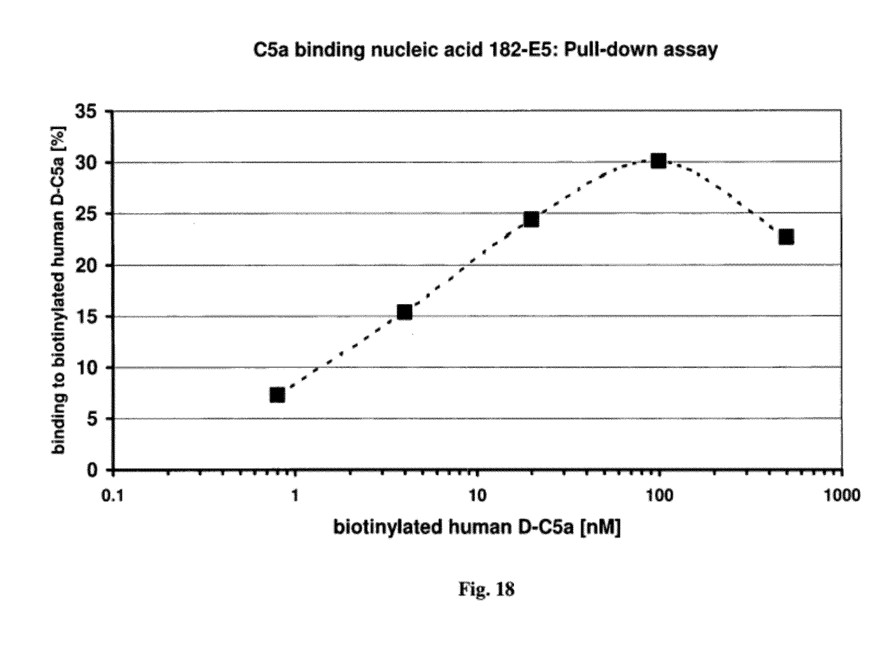

FIG. 18 shows the result of a binding analysis of the aptamer of C5a binding nucleic acid 182-E5 to biotinylated human D-05a at 37° C., represented as binding of the aptamer over concentration of biotinylated human D-05a;

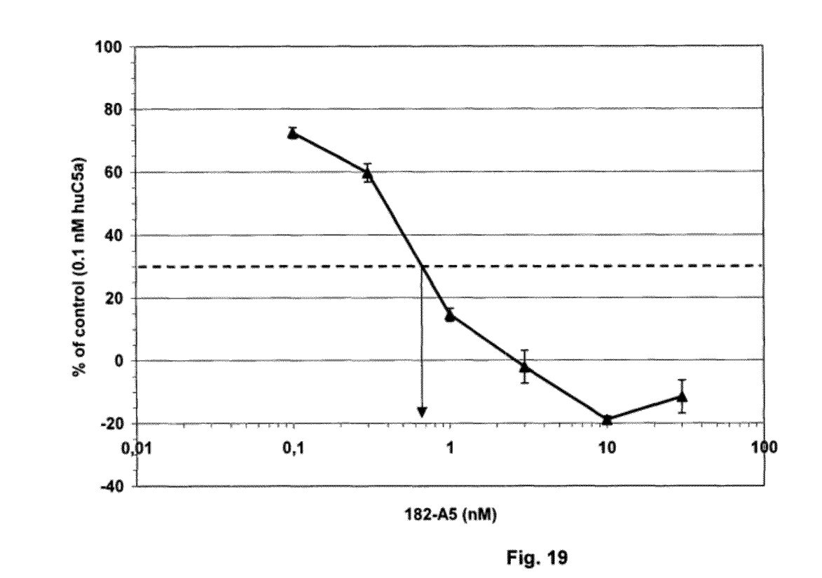

FIG. 19 shows the efficacy of Spiegelmer 182-E5 in a chemotaxis assay; cells were allowed to migrate towards 0.1 nM human C5a preincubated at 37° C. with various amounts of Spiegelmer 182-E5, represented as percentage of control over concentration of Spiegelmer 182-E5;

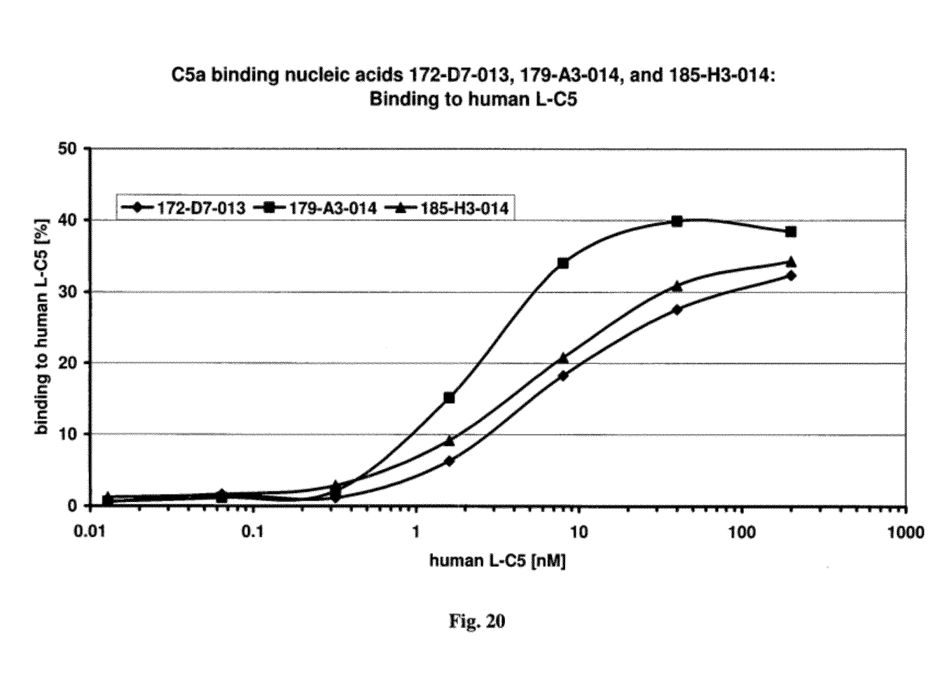

FIG. 20 shows the result of a binding analysis of the Spiegelmers (that are modified with two additional guaonsine in D-configuration at the 5′-end of the Spiegelmers whereby the 5′-end was radioactively labeled using a kinase) of C5a binding nucleic acids 172-D7-013, 179-A3-014 and 185-H3-014 to human L-05 at 37° C., represented as binding of the Spiegelmers over concentration of human L-05; and

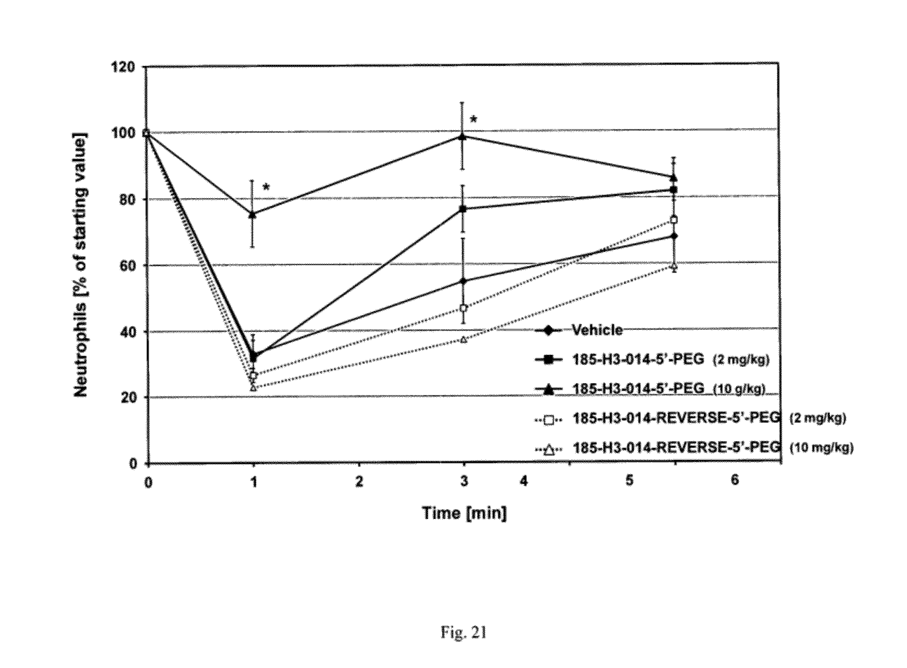

FIG. 21 shows the inhibition of C5a-induced neutropenia in mongolian gerbils, whereby the neutrophil content in gerbils following injection of C5a after application of the test substances (Spiegelmer 185-H3-014-5′-PEG or reverse Spiegelmer 185-H3-014-REVERSE-5′-PEG) and vehicle, respectively is represented over the time; whereby the test substance (Spiegelmer 185-H3-014-5′-PEG or reverse Spiegelmer 185-H3-014-REVERSE-5′-PEG) or vehicle was injected at t=−10 min i.v. in the doses indicated; whereby blood was drawn right before induction of neutropenia using 100 μg/kg rec. human C5a (i.v.); whereby further blood draws were done at 3 and 5 min after C5a injection respectively;

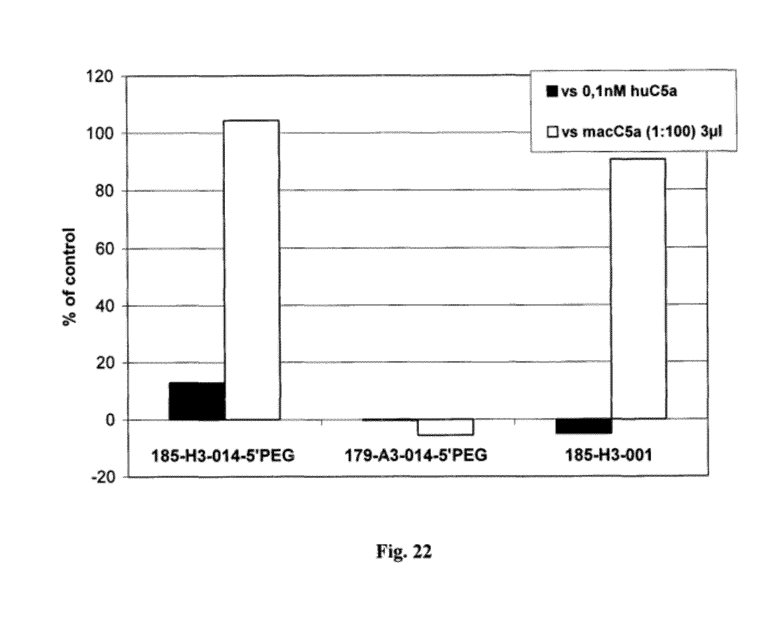

FIG. 22 shows the efficacy of Spiegelmers 185-H3-014-5′-PEG, 179-A3-014-5′-PEG and 185-H3-001 in a chemotaxis assay; cells were allowed to migrate towards 0.1 nM human C5a or 0.8 nM monkey C5a preincubated at 37° C. with 100 nM of the Spiegelmers, represented as percentage of control over concentration of Spiegelmers;

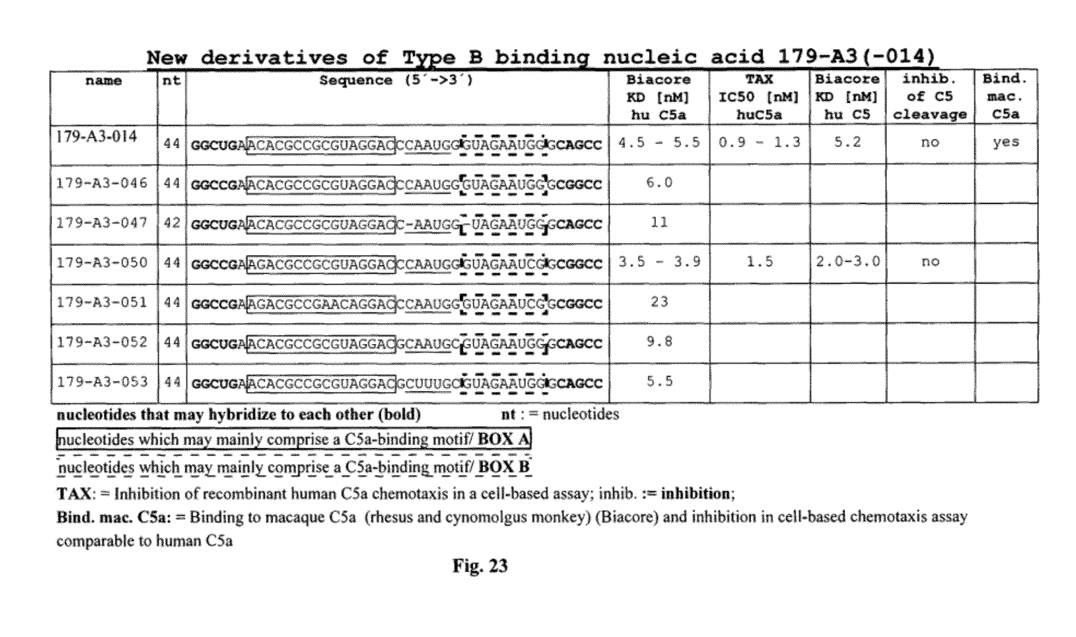

FIG. 23 shows further derivatives of RNA ligand 179-A3(-14) (human C5a RNA ligand of sequence motif “Type B”);

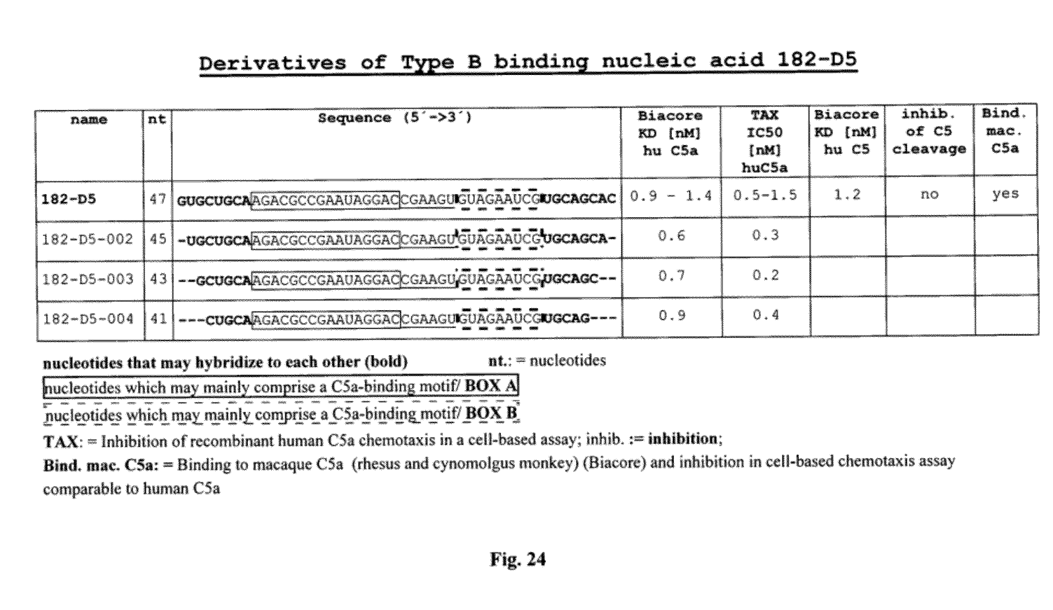

FIG. 24 shows derivatives of RNA ligand 182-D5 (human C5a RNA ligand of sequence motif “Type B”);

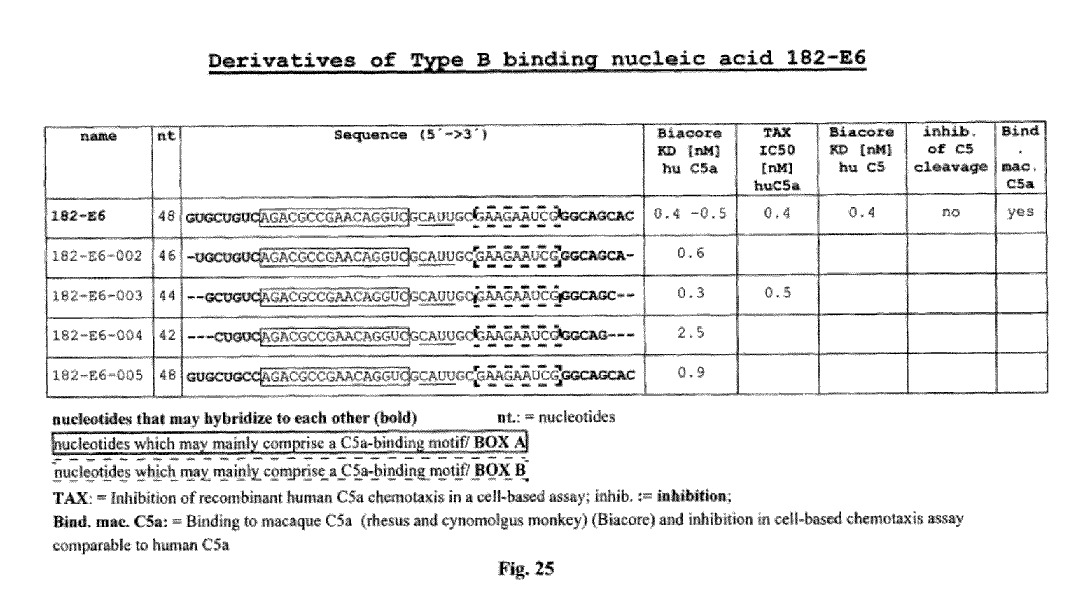

FIG. 25 shows derivatives of RNA ligand 182-E6 (human C5a RNA ligand of sequence motif “Type B”);

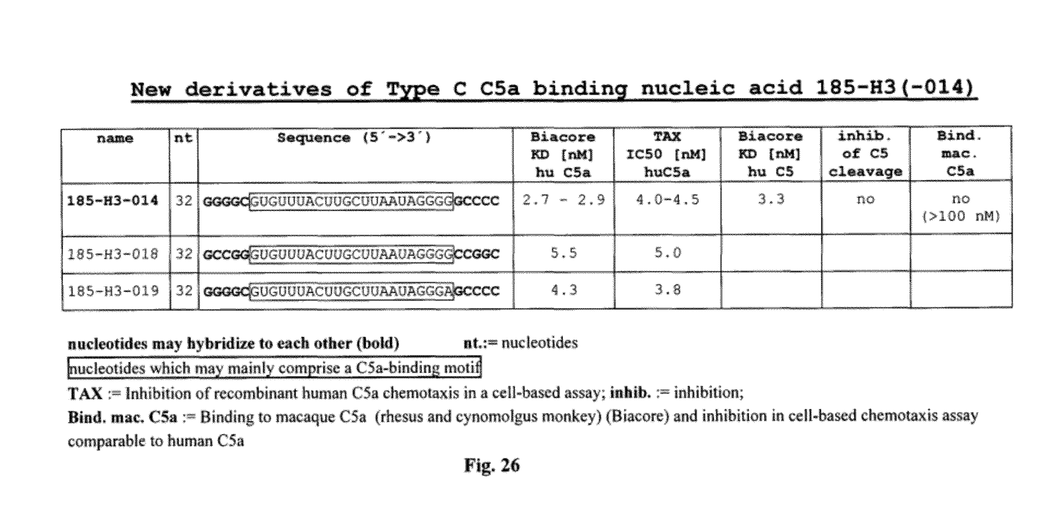

FIG. 26 shows further derivatives of RNA ligand 185-H3(-14) (human C5a RNA ligand of sequence motif “Type C”);

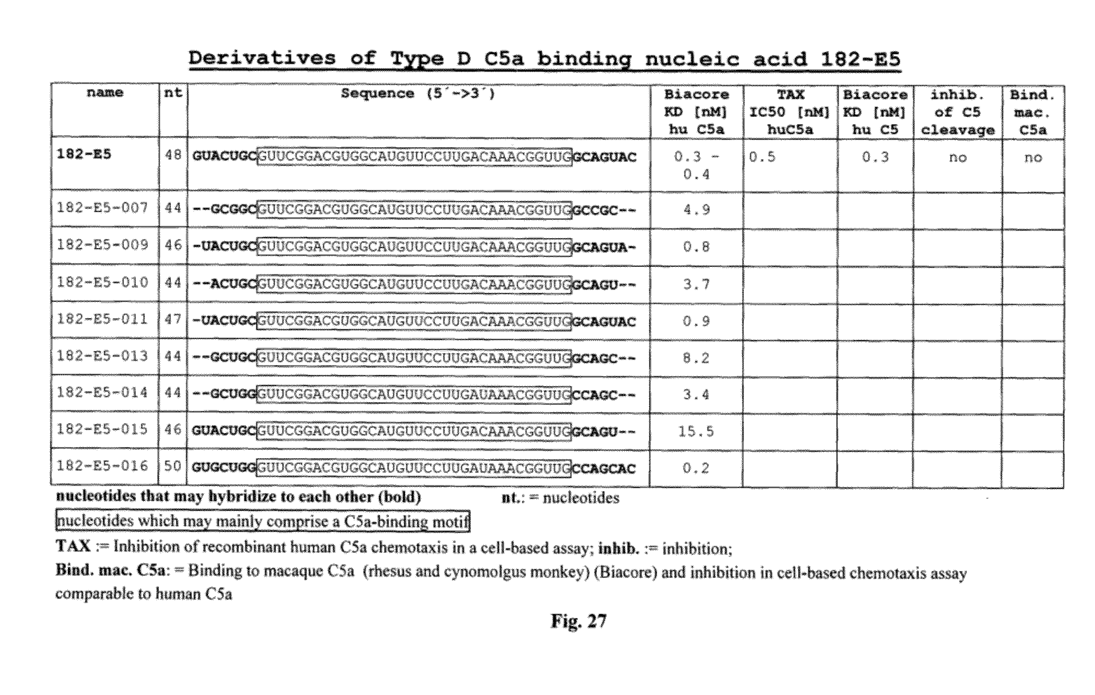

FIG. 27 shows derivatives of RNA ligand 182-E5 (human C5a RNA ligand of sequence motif “Type D”);

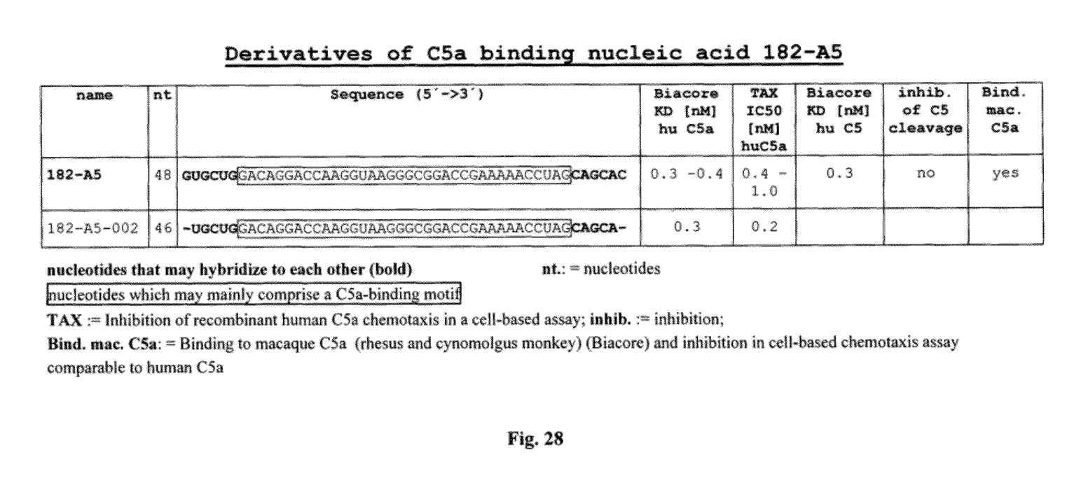

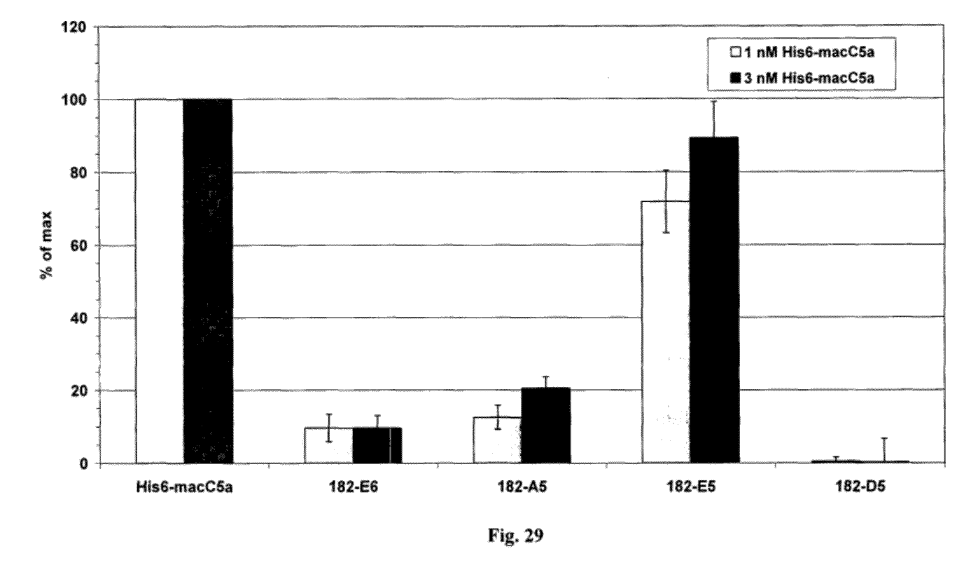

FIG. 28 shows derivatives of RNA ligand 182-A5;

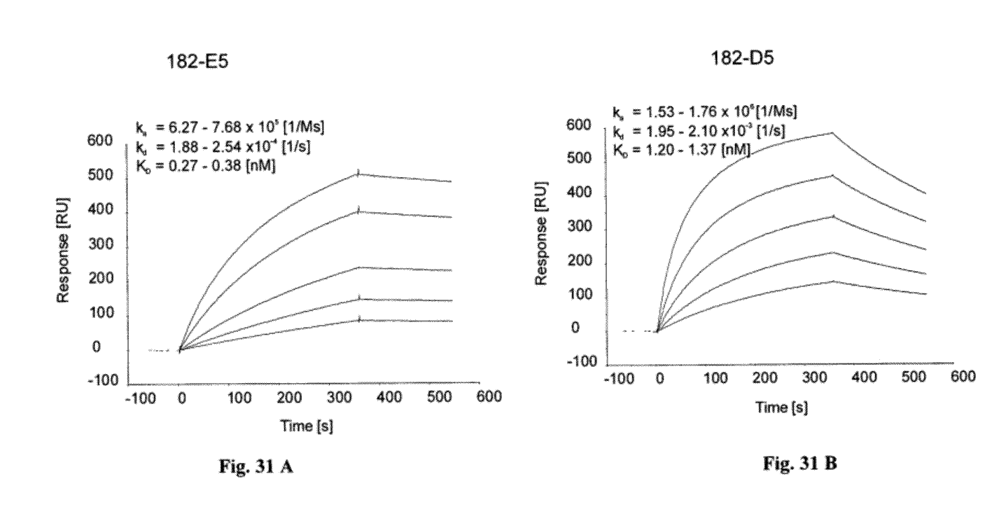

FIG. 31 shows the efficacy of Spiegelmers 182-E6, 182-A5, 182-E5 and 182-D5 in a chemotaxis assay; cells were allowed to migrate towards 1 nM or 3 nM monkey C5a preincubated at 37° C. with 100 nM of the Spiegelmers, represented as percentage of control over concentration of Spiegelmers;

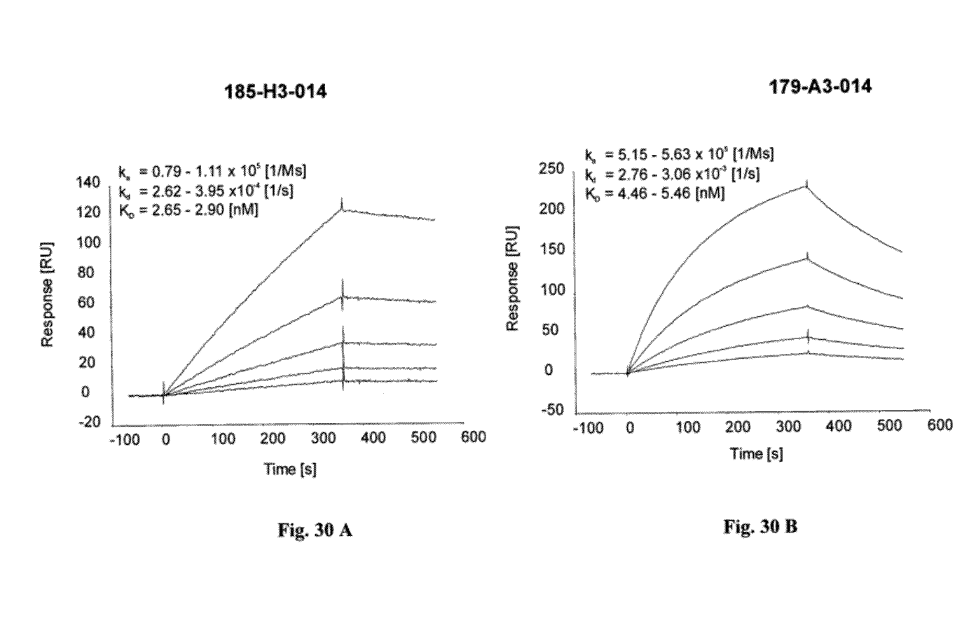

FIG. 30A shows Biacore measurement of Spiegelmer 185413-014 binding to covalently immobilized human C5a, whereby 1000-500-250-125-62.5-0 [nM] of Spiegelmers were injected in physiological buffer at a flow of 30 μl min and a temperature of 37° C.;

FIG. 30B shows Biacore measurement of Spiegelmer 179-A3-14 binding to covalently immobilized human C5a, whereby 1000-500-250-125-62.5-0 [nM] of Spiegelmers were injected in physiological buffer at a flow of 30 μl/min and a temperature of 37° C.;

FIG. 31A shows Biacore measurement of Spiegelmer 182-E5 binding to covalently immobilized human C5a, whereby 1000-500-250-125-62.5-0 [nM] of Spiegelmers were injected in physiological buffer at a flow of 30 μl/min and a temperature of 37° C.;

FIG. 31B shows Biacore measurement of Spiegelmer 182-D5 binding to covalently immobilized human C5a, whereby 1000-500-250-125-62.5-0 [nM] of Spiegelmers were injected in physiological buffer at a flow of 30 μl min and a temperature of 37° C.;

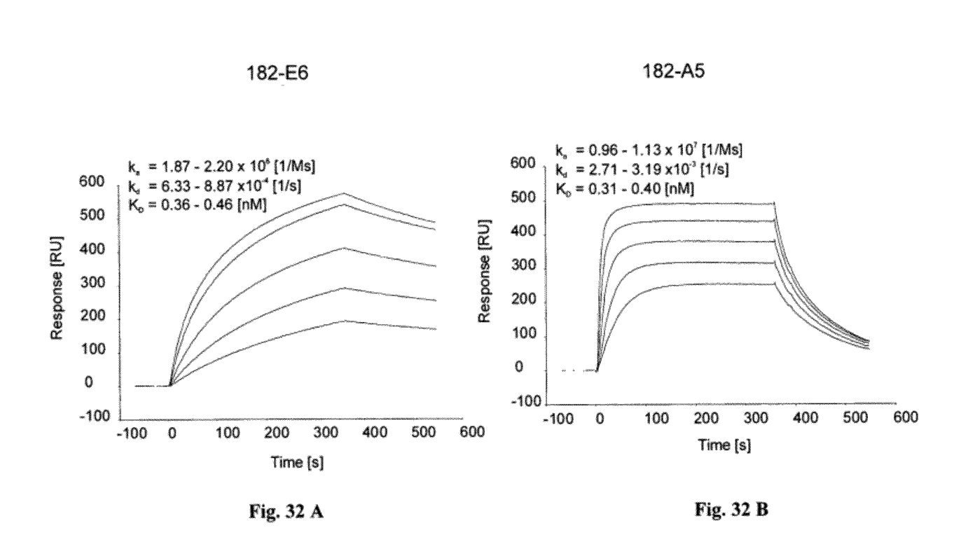

FIG. 32A shows Biacore measurement of Spiegelmer 182-E6 binding to covalently immobilized human C5a, whereby 1000-500-250-125-62.5-0 [nM] of Spiegelmers were injected in physiological buffer at a flow of 30 μl/min and a temperature of 37° C.;

FIG. 32B shows Biacore measurement of Spiegelmer 182-A5 binding to covalently immobilized human C5a, whereby 1000-500-250-125-62.5-0 [nM] of Spiegelmers were injected in physiological buffer at a flow of 30 μl min and a temperature of 37° C.;

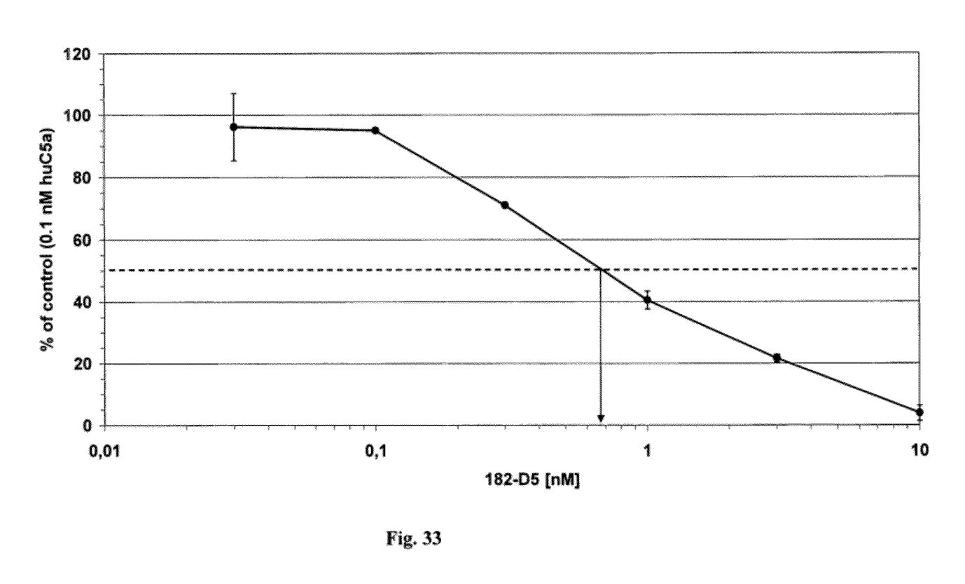

FIG. 33 shows the efficacy of Spiegelmer 182-D5 in a chemotaxis assay; cells were allowed to migrate towards 0.1 nM human C5a preincubated at 37° C. with various amounts of Spiegelmer 182-D5, represented as percentage of control over concentration of Spiegelmer 182-D5;

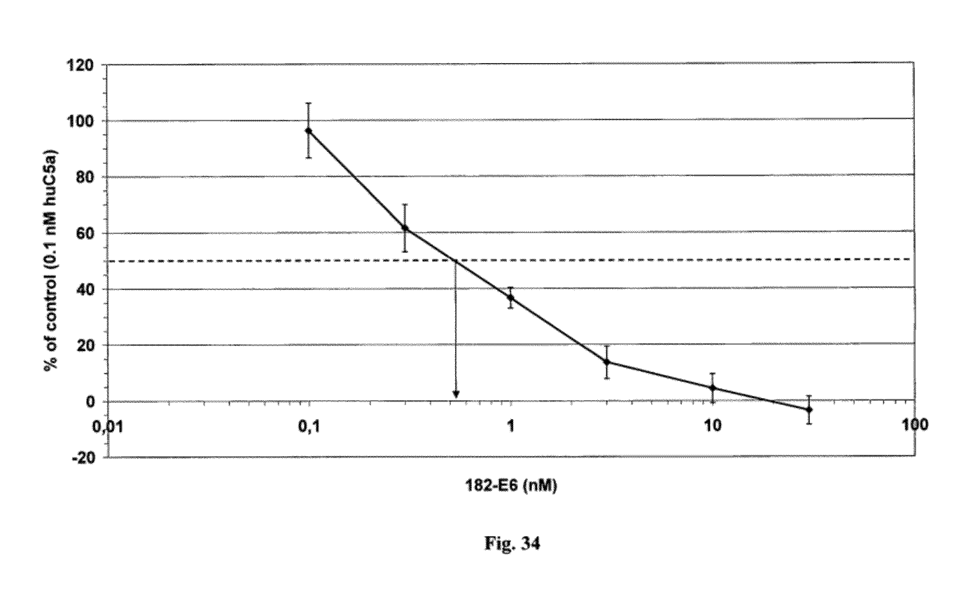

FIG. 34 shows the efficacy of Spiegelmer 182-E6 in a chemotaxis assay; cells were allowed to migrate towards 0.1 nM human C5a preincubated at 37° C. with various amounts of Spiegelmer 182-E6, represented as percentage of control over concentration of Spiegelmer 182-E6;

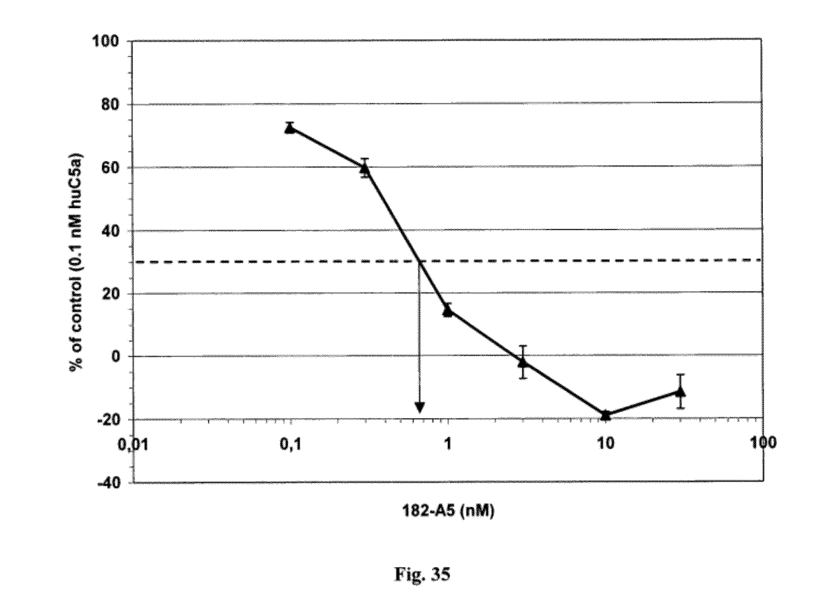

FIG. 35 shows the efficacy of Spiegelmer 182-A5 in a chemotaxis assay; cells were allowed to migrate towards 0.1 nM human C5a preincubated at 37° C. with various amounts of Spiegelmer 182-A5, represented as percentage of control over concentration of Spiegelmer 182-A5;

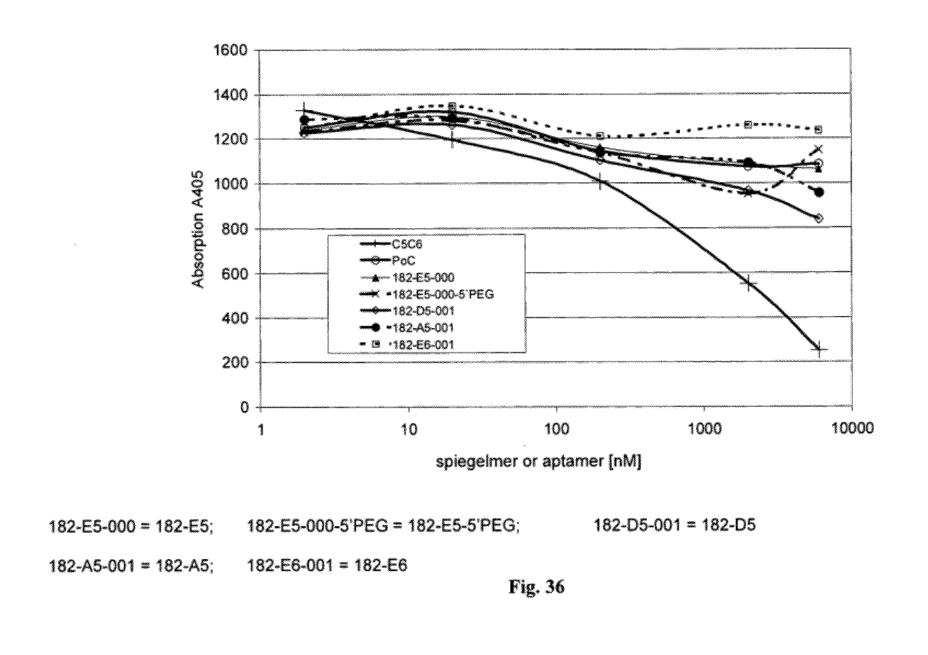

FIG. 36 shows the results of the complement C5 hemolysis test for the C5a and C5 binding spiegelmers 182-E5, 182-E5-5′PEG, 182-D5, 182-A5, 182-E6 and the non C5a and C5 binding Spiegelmer ‘PoC’ and the C5 binding aptamer C5C6

### EXAMPLE 1

### Nucleic Acids that Bind Human C5a

Using biotinylated human D-05a as a target, several nucleic acids that bind to human C5a could be generated: the nucleotide sequences of which are depicted in FIGS. 1 through 9 and 23 through 28. The nucleic acids were characterized on the aptamer, i.e. D-nucleic acid level using competitive or direct pull-down assays with biotinylated human D-05a (Example 3) or on the Spiegelmer level, i.e. L-nucleic acid with the natural configuration of human C5a (human L-05a) by an in vitro cell culture Ca²⁺-release assay (Example 4), an in vitro chemotaxis assay (Example 5) or by plasmon resonance measurement (Example 9). The Spiegelmers and aptamers were synthesized as described in Example 2.

The nucleic acid molecules thus generated exhibit different sequence motifs, four main types were identified and defined as depicted in FIGS. 1 and 2 (Type A), FIG. 3-5 and FIG. 23-25 (Type B), FIGS. 6, 7 and 26 (Type C), and FIGS. 8 and 27(Type D). Additional C5a binding nucleic acids which can not be related to each other and to the different sequence motifs described herein, are listed in FIG. 9, whereby one nucleic acid molecule thereof, the C5a binding nucleic acid molecule 182-A5 was characterized in several assay systems and derivatives thereof were synthesized and characterized (FIG. 28) For definition of nucleotide sequence motifs, the IUPAC abbreviations for ambiguous nucleotides are used:

| | | |
|---|---|---|
| S | strong | G or C; |
| W | weak | A or U; |
| R | purine | G or A; |
| Y | pyrimidine | C or U; |
| K | keto | G or U; |
| M | imino | A or C; |
| B | not A | C or U or G; |
| D | not C | A or G or U; |
| H | not G | A or C or U; |
| V | not U | A or C or G; |
| N | all | A or G or C or U |

If not indicated to the contrary, any nucleic acid sequence or sequence of stretches and boxes, respectively, is indicated in the 5′→3′ direction.

### 1.1 Type A C5a Binding Nucleic Acids

As depicted in FIG. 1 and FIG. 2 all sequences of C5a binding nucleic acids of Type A comprise one central sequence stretch or box defining a potential C5a binding motif which is flanked by 5′- and 3′-terminal stretches that can hybridize to each other. Within the central sequence stretch some nucleotides can hybridize to each other, too. However, such hybridization is not necessarily given in the molecule. Moreover, at single positions of the central sequence stretch one or more of the nucleotides can be replaced by a hydrophilic spacer, e.g. by a C18-PEG spacer.

It is within the present invention that—with regard to Type A C5a binding nucleic acids—the terms ‘5′-terminal stretch’ and ‘first terminal stretch’, ‘central sequence’ and ‘central stretch’ and ‘central box’, and ‘3′-terminal stretch’ and “second terminal stretch’, respectively are used herein in a synonymous manner if not indicated to the contrary.

The nucleic acids were characterized on the aptamer level using direct and competitive pull-down binding assays with biotinylated human D-05a in order to rank them with respect to their binding behaviour (Example 3). Selected sequences were synthesized as Spiegelmers (Example 2) and were tested using the natural configuration of human C5a (human L-05a) in a cell culture in vitro Ca²⁺-assay (Example 4) or a chemotaxis assay (Example 5).

The sequences of the defined boxes or stretches may be different between the C5a binding nucleic acids of Type A which influences the binding affinity to human C5a. Based on binding analysis of the different C5a binding nucleic acids summarized as Type A C5a binding nucleic acids, the central box and its nucleotide sequences as described in the following are individually and more preferably in their entirety essential for binding to human C5a:

The central box of all identified sequences of Type A C5a binding nucleic acids share the central sequence

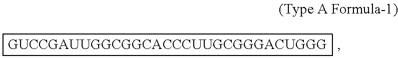

whereby within the central sequence stretch some nucleotides can hybridize to each other (marked as bold and italic letters) and at single positions of the central sequence stretch one or more of the nucleotides can be replaced by a hydrophilic spacer, e.g. by a C18-PEG spacer.

The nucleotides within the central sequence stretch that can hybridize to each other are two substretches of three nucleotides, respectively, whereby the first substretch comprise the nucleotides at position 16 to 18 and the second substretch comprise the nucleotides 23 to 25. The sequence of the three nucleotides of the first and the second substretch is independently CCC or GGG, whereby the sequence of the first and the second substretch is different but in any case the first and the second substretch are complementary to each other.

The origin of all Type A C5a binding nucleic acids is the Type A C5a binding nucleic acid 172-D7-000 that was characterized for its binding affinity to human C5a in several different assays. The equilibrium binding constant K_{D} was determined using the pull-down binding assay (K_{D=30} nM, FIG. 10). The IC₅₀ (inhibitory concentration 50%) of 2-3 nM for Type A C5a binding nucleic acid 172-D7-000 was measured using a cell culture Ca²⁺-release. Derivatives of Type A C5a binding nucleic acid 172-D7-000 were analyzed as aptmers by using the pull-down assay (determination of the binding constant K_{D}) or in comparison to Type A C5a binding nucleic acid 172-D7-000 by using the competition assay.

Nine nucleotides of the 5′-terminal stretch of Type A C5a binding nucleic acid 172-D7-000 may hybridize to the respective nine nucleotides of the 3′-terminal stretch to form a terminal helix of nine base-pairing nucleotides. However, the 3′ terminal nucleotide ‘U’ of 5′-terminal stretch can not be replaced by an ‘C’ without reduction of binding activity (172-D7-003; K_{D}=372 nM). As firstly shown for the derivatives 172-D7-001, 172-D7-010 and 172-D7-011 of Type A C5a binding nucleic acid 172-D7-000, a helix of seven base pairs seemed to be sufficient in order to maintain C5a binding activity. If the central sequence stretch was flanked by only six nucleotides at the 5′- and the 3′-end (5′-end: ‘GUGCUU’; 3′-end: ‘GAGUAC’) forming a helix with six base pairs), the binding affinity was reduced (172-D7-002; K_{D}=108 nM). Surprisingly, later experiments revealed that a helix of six base pairs formed by ‘GCGCUU’ of the 5′-terminal stretch and by ‘GAGCGC’ of the 3′-terminal stretch is sufficient for forming a fully active structure of Type A C5a binding nucleic acids (172-D7-012, 172-D7-013, 172-D7-014). A reduction to five nucleotides for the 5′- and 3′-terminal stretch may have a negative effect on forming the fully active three-dimensional structure of Type A C5a binding nucleic acids (172-D7-017).

However, combining the 5′- and 3′-terminal stretches of all tested Type A C5a binding nucleic acids the generic formula for the 5′-terminal stretch of Type A C5a binding nucleic acids is 5′ X₁X₂X₃GYGCX₄Y 3′ (Type A Formula-2-5′) and the generic formula for the 3′-terminal stretch Type A C5a binding nucleic acids is 5′ GX₅GYRCX₆X₇X₈ 3′ (Type A Formula-2-3′), whereas X₁ is A or absent, X₂ is G or absent, X₃ is C or absent, X₄ is U, X₅ is A, X₆ is G or absent, X₇ is C or absent, and X₈ is U or absent, or

X₁ is A or absent, X₂ is G or absent, X₃ is C or absent, X₄ is absent, X₅ is absent, X₆ is G or absent, X₇ is C or absent, and X₈ is U or absent.

As mentioned above, a helix of six or seven base pairs seemed to be sufficient in order to maintain C5a binding activity. Therefore, the preferred 5′- and 3′-terminal stretches are specified by the generic formula for the 5′-terminal stretch of Type A C5a binding nucleic acids 5′ X₁X₂X₃GYGCX₄Y 3′ (Type A Formula-2-5′) and the generic formula for the 3′-terminal stretch Type A C5a binding nucleic acids is 5′ GX₅GYRCX₆X₇X₈ 3′ (Type A Formula-2-3′), whereby X₁ is absent, X₂ is absent, X₃ is C or absent, X₄ is U, X₅ is A, X₆ is G or absent, X₇ is absent, and X₈ is absent.

The best binding affinities can be achieved in the case of 5′- and 3′-terminal stretches that are specified by the generic formula for the 5′-terminal stretch of Type A C5a binding nucleic acids Type A Formula-3-5′ (5′ X₃GYGCX₄U 3′) and the generic formula for the 3′-terminal stretch Type A C5a binding nucleic acids Type A Formula-3-3′ (5′ GX₅GYGCX₆ 3′), whereby X₃ is C or absent, X₄ is U, X₅ is A, and X₆ is G or absent.

Another strategy to reduce the number of nucleotides was to replace some nucleotides within the central sequence stretch of Type A C5a binding nucleic acids by a C18-PEG spacer. Within the central sequence stretch respectively three nucleotides can hybridize to each other, potentially forming a helix. As shown for derivatives 172-D7-005, 172-D7-008, 172-D7-009, 172-D7-013 and 172-D7-014 the four nucleotides that are flanked by the helix in the central sequence stretch of Type A C5a binding nucleic acids can be replaced by a C18-PEG spacer without significant reduction of the molecule's binding affinity to C5a. Deletion of one out of the three nucleotides forming a helix within the central sequence stretch led to a reduction of binding affinity (172-D7-018). Other sequence segments of the central stretch of Type A C5a binding nucleic acids are much more sensitive concerning replacement strategies as described above. Hence, the derivatives that were designed to determine this option showed reduced binding affinity to C5a (172-D7-004, 172-D7-015, 172-D7-016).

For the PEGylated derivative of C5a binding nucleic acid 172-D7-013, 172-D7-013-5′-PEG, an IC₅₀ of approx. 6.5 nM was determined in the Ca⁺⁺-release assay (FIG. 11).

### 1.2 Type B C5a Binding Nucleic Acids

As depicted in FIGS. 3.-5 and 23-25 all sequences of C5a binding nucleic acids of Type B comprise two highly conserved sequence stretches or boxes—Box A and Box B—which are linked to each other by a stretch of up to eleven nucleotides—called Box L—and flanked by 5′- and 3′-terminal stretches that can hybridize to each other. Within the Box L some nucleotides can hybridize to each other, too. However, such hybridization is not necessarily given in the molecule. Moreover, at single positions of the Box L one or more of the nucleotides can be replaced by a hydrophilic spacer, e.g. by a C18-PEG spacer.

It is within the present invention that—with regard to Type B C5a binding nucleic acids—the terms ‘5′-terminal stretch’ and ‘first terminal stretch’, ‘Box A’ and ‘first central stretch’, ‘Box L’ and ‘second central stretch, ‘Box B’ and ‘third central stretch’, and ‘3′-terminal stretch’ and ‘second terminal stretch’, respectively are used herein in a synonymous manner if not indicated to the contrary.

The nucleic acids were characterized on the aptamer level using direct and competitive pull-down binding assays with biotinylated human D-05a in order to rank them with respect to their binding behaviour (Example 3). Selected sequences were synthesized as Spiegelmers (Example 2) and were tested using the natural configuration of human C5a (human L-05a) in a chemotaxis assay (Example 5) or by plasmon resonance measurement (Example 9).

The sequences of the defined boxes or stretches may be different between the C5a binding nucleic acids of Type B which influences the binding affinity to human C5a. Based on binding analysis of the different C5a binding nucleic acids summarized as Type B C5a binding nucleic acids, the sequence stretches or boxes and its nucleotide sequences as described in the following are individually and more preferably in their entirety essential for binding to human C5a:

Type B C5a binding nucleic acids comprise two highly conserved sequence stretches—Box A and Box B—defining a potential C5a binding motif. Box A and Box B are linked to each other by up to eleven nucleotides, called ‘Box L’. The such manner linked sequence stretches Box A and Box B are flanked by 5′- and 3′-terminal stretches that can hybridize to each other. Between the 5′-terminal stretch and Box A and between the 3′-terminal stretch and Box B none up to four additional nucleotides can be located. These nucleotides seem not hybridize to each other or to other nucleotides within the Type B C5a binding nucleic acid molecules.

The Box A of all identified sequences of Type B C5a binding nucleic acids share the consensus sequence

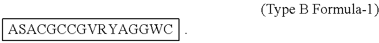

The consensus sequence of Box B for Type B C5a binding nucleic acids is

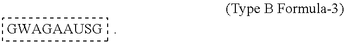

However, C5a binding nucleic 179-A3-047 (FIG. 23) has a Box B with the sequence of UAGGAUGG leading to a consensus sequence of for Box B of WAGAAUSG. In order to determine the binding affinities of the different Type B C5a binding nucleic acids 179-A3, 179-C1, 179-D3, 179-E1, 179-A4, 182-E6, 179-G1, 182-D5, 179-F2 to human C5a they were tested on the aptamer level using direct and competitive pull-down binding assays with biotinylated human D-05a (Example 3). As reference the Type A C5a binding nucleic acid 172-D7-000 was used. (K_{D}=30 nM, IC₅₀=2-3 nM). Type B C5a binding nucleic acids 179-A3, 179-C1, 179-D3, 179-E1, 182-E6 and 182-D5 showed almost similar binding affinity to human C5a, whereby the binding affinity is better than the binding affinity of Type A C5a binding nucleic acid 172-D7-000. Type B C5a binding nucleic acids 179-A4, 179-G1 and 179-F2 showed similar binding to human C5a as Type A C5a binding nucleic acid 172-D7-000. Because the Box A sequences of Type B C5a binding nucleic acids 179-F2 (Box A:

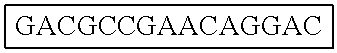

### ) and 179-G1 (Box A:

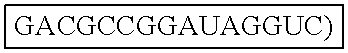

are different from the Type B C5a binding nucleic acids with the best affinity to C5a, viz. Type B C5a binding nucleic acids 179-A3, 179-C1 and 179-D3, the preferred consensus sequence of Box A for Type B C5a binding nucleic acids is

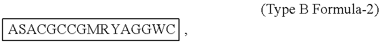

whereby the preferred consensus sequence of Box A for Type B C5a binding nucleic acids results from the Box A sequences of Type B C5a binding nucleic acids 179-A3, 179-C1 and 179-D3.

The nucleotides of Boxes A and B of Type B C5a binding nucleic acids interacts in a sequence-specific manner. If the second nucleotide at the 5′-end of Box A is ‘C’ then the corresponding nucleotide in Box B is ‘G’ (the nucleotide next to the last at the 3′-end of Box B; see 179-A3 and 179-C1). Alternatively, the second nucleotide at the 5′-end of Box A is ‘G’ and the corresponding nucleotide in Box B is ‘C’ (the nucleotide next to last at the 3′-end of Box B; see 179-D3, 179-E1, 179-A4, 182-E6, 179-G1, 182-D5, 179-F2). In addition, if the nucleotide next to last at the 3′-end of Box A is ‘A’ then the corresponding nucleotide in Box B is ‘U’ (the second nucleotide at the 5′-end of box B; see 179-A3, 182-D5 and 179-F2). Alternatively, the nucleotide next to last at the 3′-end of box A is ‘U’ and the corresponding nucleotide in Box B is ‘A’ (the second nucleotide at the 5′-end of Box B; see 179-C1, 179-D3, 179-E1, 179-A4, 182-E6 and 179-G1).

The 3′-end of Box A is linked to the 5′-end of Box B by up to eleven nucleotides—called ‘Box L’—whereby the central nucleotides of the Box L are not hybridized to each other and thereby form a so called ‘loop’-structure. Three up to seven nucleotides can form such a ‘loop’-structure. The additional nucleotides that do not form the ‘loop’-structure hybridize to each other and/or to the 3′-end of Box A and the 5′-end of Box B, respectively. The respective sequences of the linking boxes (Box L) of the Type B C5a binding nucleic acids are very different to each other whereby the sequence and number of nucleotides are highly variable (see FIG. 3). On basis of the Type B C5a binding nucleic acid 179-A3 different derivatives were designed and tested (FIGS. 4 and 5). As shown for Type B C5a binding nucleic acid 179-A3-014, two nucleotides could be deleted without any reduction of binding affinity to human C5a. Moreover, if further three nucleotides that are part of the loop were replaced by a C18-PEG-spacer the molecule 179-A3-042 was as active as the original molecule 179-A3-014. As shown for Type B C5a binding nucleic acid 179-A3-042 the Box L comprises a first and a second substretch, whereby the first and the second substretch hybridize to each other. In the case of hybridization a double-stranded structure is formed. The minimal sequence of the first and the second substretch is independently CC or GG, whereby the sequence of the first and the second substretch is different for the first and the second substretch. However, as consequence of these results, presumably the nucleotides of Box L are not responsible for binding to human C5a, but important in order to arrange Box A and Box B to each other.

Type B C5a binding nucleic acids comprise at the 5′-end and at the 3′-end four to eight nucleotides, respectively, that can hybridize to each other forming a helix. In order to truncate the molecule Type B C5a binding nucleic acid 179-A3 (K_{D}=7.2 nM, FIG. 12; IC₅₀=0.9 nM, FIG. 13) several derivatives with a different number of nucleotides and different nucleotide sequences (179-A3-014, 179-A3-003, 179-A3-007, 179-A3-008) were tested in competition experiments vs. Type B C5a binding nucleic acid 179-A3. On basis of the sequences present as 5′- and 3′-terminal stretch of Type B C5a binding nucleic acid 179-A3 the truncation down to three nucleotides at the 5′-end and the 3′-end, respectively, of the molecule led to a reduction of binding affinity (see 179-A3-008). On basis of derivative 179-A3-014 that shows identical binding affinity as the original molecule Type B C5a binding nucleic acid 179-A3 further helix arrangements at the 5′-end and the 3′-end of the molecule were tested (179-A3-015, 179-A3-020, 179-A3-021, 179-A3-024, 179-A3-026, 179-A3-029, 179-A3-030, 179-A3-034, 179-A3-037). In competition experiments versus Type B C5a binding nucleic acid 179-A3-014 it could be shown that minimal four nucleotides at both ends that hybridize to each other are essential for a fully active structure of a Type B C5a binding nucleic acid (179-A3-030, 5′-end: CGCC, 3′-end: GGCG; 179-A3-034, 5′-end: CCGG, 3′-end: CCGG). Furthermore Type B C5a binding nucleic acid 179-A3-007 (5′-end: GCUG, 3′-end: CAGC) is a fully active derivative of Type B C5a binding nucleic acid 179-A3.

However, combining the 5′- and 3′-terminal stretches of all tested Type B C5a binding nucleic acids (as depicted in FIGS. 3, 4 and 5) the generic formula for the 5′-terminal stretch of Type B C5a binding nucleic acids is 5′ X₁X₂SBBX₃X₄X₅ 3′ (Type B Formula-4-5′) and the generic formula for the 3′-terminal stretch Type B C5a binding nucleic acids is 5′ X₆X₇X₈VVSX₉X₁₀ 3′ (Type B Formula-4-3′),

whereby
X₁ is G or absent, X₂ is U or absent, X₃ is B, X₄ is Y, X₅ is M, X₆ is K, X₇ is G, X₈ is N, X₉ is A or absent, and X₁₀ is C or absent,
or
X₁ is G or absent, X₂ is U or absent, X₃ is B, X₄ is Y, X₅ is absent, X₆ is absent, X₇ is G, X₈ is N, X₉ is A or absent, and X₁₀ is C or absent,
or
X₁ is G or absent, X₂ 1S U or absent, X₃ is absent, X₄ is Y, X₅ is M, X₆ is K, X₇ is G, X₈ is absent, X₉ is A or absent, and X₁₀ is C or absent,
or
X₁ is G or absent, X₂ is U or absent, X₃ is B, X₄ is absent, X₅ is M, X₆ is K, X₇ is absent, X₈ is N, X₉ is A or absent, and X₁₀ is C or absent,
or
X₁ is G or absent, X₂ is U or absent, X₃ is B, X₄ is absent, X₅ is absent, X₆ is absent, X₇ is absent, X₈ is N, X₉ is A or absent, and X₁₀ is C or absent,
or
X₁ is G or absent, X₂ is U or absent, X₃ is absent, X₄ is absent, X₅ is M, X₆ is K, X₇ is absent, X₈ is absent, X₉ is A or absent, and X₁₀ is C or absent,
or
X₁ is G or absent, X₂ is U or absent, X₃ is absent, X₄ is Y, X₅ is absent, X₆ is absent, X₇ is G, X₈ is absent, X₉ is A or absent, and X₁₀ is C or absent,
or
X₁ is G or absent, X₂ is U or absent, X₃ is absent, X₄ is absent, X₅ is absent, X₆ is absent, X₇ is absent, X₈ is absent, X₉ is A or absent, and X₁₀ is C or absent.

As mentioned above, a helix of four to six base pairs seemed to be sufficient in order to maintain C5a binding activity as shown for Type B C5a binding nucleic acid 179-A3 and its derivatives. Therefore, the preferred 5′- and 3′-terminal stretches can be specified by the generic formula for the 5′-terminal stretch of Type B C5a binding nucleic acids 5′ X₁X₂SSBX₃X₄X₅ 3′ (Type B Formula-7-5′) and the generic formula for the 3′-terminal stretch Type B C5a binding nucleic acids 5′ X₆X₇X₈VSSX₉X₁₀ 3′ (Type B Formula-7-3′), whereby X₁ is G or absent, X₂ is U or absent, X₃ is S, X₄ is absent, X₅ is absent, X₆ is absent, X₇ is absent, X₈ is S, X₉ is A or absent, and X₁₀ is C or absent, whereby preferably X₁ is absent, X₂ is absent, X₃ is S, X₄ is absent, X₅ is absent, X₆ is absent, X₇ is absent, X₈ is S, X₉ is absent, and X₁₀ is absent.

The best binding affinities of Type B C5a binding nucleic acids comprising 5′- and 3′-terminal stretches with four nucleotides, are shown for Type B C5a binding nucleic acids 179-A3-030 (5′-end: CGCC, 3′-end: GGCG), 179-A3-034 (5′-end: CCGG, 3′-end: CCGG) and 179-A3-007 (5′-end: GCUG, 3′-end: CAGC).

However, Type B C5a binding nucleic acid 179-C1 and its potential derivatives can be specified by the generic formula for the 5′-terminal stretch of Type B C5a binding nucleic acids 5′ X₁X₂GCYX₃X₄X₅ 3′ (Type B Formula-5-5′) and the generic formula for the 3′-terminal stretch Type B C5a binding nucleic acids is 5′ X₆X₇X₈AGCX₉X₁₀ 3′. (Type B Formula-5-3′), whereby X₁ is G or absent, X₂ is U or absent, X₃ is G, X₄ is C, X₅ is absent, X₆ is absent, X₇ is G, X₈ is C, X₉ is A or absent, and X₁₀ is C or absent.

Moreover, Type B C5a binding nucleic acid 179-D3 and its potential derivatives can be specified by the identical generic formula for the 5′-terminal stretch of Type B C5a binding nucleic acids 5′ X₁X₂GCCX₃X₄X₅ 3′ (Type B Formula-6-5′) and the generic formula for the 3′-terminal stretch Type B C5a binding nucleic acids is 5′ X₆X₇X₈AGCX₉X₁₀ 3′. (Type B Formula-5-3′), whereby X₁ is G or absent, X₂ is U or absent, X₃ is G, X₄ is C, X₅ is C, X₆ is G, X₇ is G, X₈ is C, X₉ is A or absent, and X₁₀ is C or absent.

The 3′-end of 5′-terminal helix forming sequence stretch is linked to the 5′-end of Box A by zero to four nucleotides, whereby these one to five nucleotides do not hybridize to other nucleotides within the Type B C5a binding nucleic acid molecules. Additionally, the 3′-end of Box B is linked to 5′-end of 3′-terminal helix forming sequence stretch by zero or one nucleotides, whereby these one or two nucleotides do not hybridize to other nucleotides within the Type B C5a binding nucleic acid molecules. These not hybridized nucleotides 5′ of the 5′-end of Box A and 3′ of the 3′-end of Box B preferably are either not existent or ‘A’ and ‘G’. (true for all Type B C5a binding nucleic acids as listed in FIG. 3-5, except Type B C5a binding nucleic acid 179-G1).

For the PEGylated derivative of C5a binding nucleic acid 179-A3-014, 179-A3-014-5′-PEG, an IC₅₀ of approx. 1.8 nM was determined in the TAX assay (FIG. 14).

In order to get more substantiated data about the binding affinity of Type B C5a binding nucleic acids to human C5a the best Type B C5a binding nucleic acids 179-A3-014, 182-E5 and 182-E6 were analysed as spiegelmers by plasmon resonance measurement. As K_{D}'s the following values were determined:

### 179-A3-014: K_{D} of 4.5-5.5 nM

### 182-D5: K_{D} of 0.9-1.4 nM

### 182-E6: K_{D} of 0.4 to 0.5 nM

In order to achieve more potent and shorter derivatives of Type B C5a binding nucleic acid 179-A3-014 new sequences of the central and/or 5′/3′ terminal stretches of the Type B C5a binding nucleic acids 179-A3-014 were designed, the new derivatives synthesized as spiegelmers and tested for their binding affinity to human C5a by plasmon resonance measurement. For Type B C5a binding nucleic acid 179-A3-050 (K_{D} of 3.5-3.9 nM) a slightly improved binding affinity over Type B C5a binding nucleic acid 179-A3-014 (K_{D} of 4.5-5.5 nM) could be determined. This result was supported by improved binding to human C5. The improvement of binding affinity of Type B C5a binding nucleic acid 179-A3-050 over Type B C5a binding nucleic acid 179-A3-014 is caused by a replacement of a ‘UA’-base pair by an ‘CG’-base pair in the terminal stretches, the ‘C’ to ‘G’ replacement in Box A and the ‘G’ to ‘C’ replacement in Box B (FIG. 23, 30B).

In order to achieve shorter derivatives of Type B C5a binding nucleic acid 182-D5 the number of nucleotides of the terminal stretches of the Type B C5a binding nucleic acid 182-D5 were reduced, the respective derivatives were synthesized as spiegelmers and tested for their binding affinity to human C5a by plasmon resonance measurement and their inhibitory potency in a TAX-assay. All derivatives of C5a binding nucleic acid 182-D5 share almost the same affinity (K_{D} of 0.6 to 0.9 nM) and inhibitory potency (IC₅₀ between 0.2 and 0.5 nM). Thus, the reduction of the 5′/3′-terminal stretches from 8 nucleotides down to five nucleotides was successful (FIG. 24, 31B, 33)

In order to achieve shorter derivatives of Type B C5a binding nucleic acid 182-E6 the number of nucleotides of the terminal stretches of the Type B C5a binding nucleic acid 182-D5 were reduced, the respective derivatives were synthesized as spiegelmers and tested for their binding affinity to human C5a by plasmon resonance measurement. The derivatives of C5a binding nucleic acid 182-D5 sC5a binding nucleic acids 182-E6-002 and 182-E6-003 share almost the same affinity (K_{D} of 0.3 to 0.6 nM) and inhibitory potency (IC₅₀ between 0.4 and 0.5 nM). Thus, the reduction of the 5′/3′-terminal stretches from 8 nucleotides down to six nucleotides was successful (FIG. 25, 32A, 34)

### 1.3 Type C C5a Binding Nucleic Acids

As depicted in FIG. 6 and FIG. 7 all sequences of C5a binding nucleic acids of Type C comprise one central sequence stretch or box defining a potential C5a binding motif which is flanked by 5′- and 3′-terminal stretches that can hybridize to each other. However, such hybridization is not necessarily given in the molecule.

It is within the present invention that—with regard to Type C C5a binding nucleic acids—the terms ‘5′-terminal stretch’ and ‘first stretch’, ‘central sequence’ and ‘second stretch’, and ‘3′-terminal stretch’ and ‘third stretch’, respectively are used herein in a synonymous manner if not indicated to the contrary.

The nucleic acids were characterized on the aptamer level using direct and competitive pull-down binding assays with biotinylated human D-05a in order to rank them with respect to their binding behaviour (Example 3). Selected sequences were synthesized as Spiegelmers (Example 2) and were tested using the natural configuration of human C5a (human L-05a) in a cell culture in vitro Ca²⁺-assay (Example 4) or a chemotaxis assay (Example 5).

The sequences of the defined boxes or stretches may be different between the C5a binding nucleic acids of Type C which influences the binding affinity to human C5a. Based on binding analysis of the different C5a binding nucleic acids summarized as Type C C5a binding nucleic acids, the central box and its nucleotide sequences as described in the following are individually and more preferably in their entirety essential for binding to human C5a:

The central box of all identified sequences of Type C C5a binding nucleic acids share the central sequence

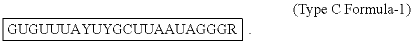

In order to determine the binding affinities of the different Type C C5a binding nucleic acids 185-H3-001, 185-D3, 185-B3, 185-B1, 184-F4, 185-A3, 185-B4, 185-G4, 185-H4 and 185-C3 to human C5a they were tested on the aptamer level using direct and competitive pull-down binding assays with biotinylated human D-05a (Example 3). As reference the Type B C5a binding nucleic acid 179-A3-015 (K_{D}>7.2 nM) or Type C C5a binding nucleic acid 185-H3-001 (K_{D}=5 nM, IC₅₀=1-3 nM, FIG. 15) was used. Type C C5a binding nucleic acid 185-H3-001 has much better binding affinity to human C5a than Type B C5a binding nucleic acid 179-A3-015. Type C C5a binding nucleic acids 185-D3, 185-B3 184-B4 and 185-G4 showed almost similar binding affinity to human C5a, whereby the binding affinity is similar to the binding affinity of Type B C5a binding nucleic acid 179-A3-015. Because Type C C5a binding nucleic acids 185-H3-001 showed the best binding affinity of Type C C5a binding nucleic acids, the preferred sequence of the central sequence for Type C C5a binding nucleic acids is

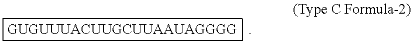

This consensus sequence Type C Formula-2 for the central sequence stretch is additionally characteristic for 185-D3, 185-B3, 185-B4 and 185-G4. Because Type C C5a binding nucleic acids 185-D3, 185-B3, 185-B4 and 185-G4 have weaker binding affinity to human C5a than Type C C5a binding nucleic acid 185-H3-001, their different binding behaviour in comparison to Type C C5a binding nucleic acid 185-H3-001 has to be founded in the different sequences of the 5′- and 3′-terminal stretches (see below).

Seven or eight nucleotides of the 5′-terminal stretch of Type C C5a binding nucleic acids can hybridize to the respective seven or eight nucleotides of the 3′-terminal stretch to potentially form a terminal helix of seven or eight base-pairing nucleotides. Although the nucleotides are variable at several positions (see FIG. 6), the different nucleotides allow for hybridization of seven or eight nucleotides of the 5′- and 3′-terminal stretches each, whereby as shown for Type C C5a binding nucleic acids 185—H3-001, 185-D3, 185-B3, 185-B4 and 185-G4, that have the identical Box A, the sequence of the 5′- and 3′-terminal stretch has an influence of the binding behaviour to C5a (FIG. 6) Additionally, truncated derivatives of Type C C5a binding nucleic acids 185-H3-001 and 185-B4 (both sequences comprise the same central sequence) were analyzed in a competitive pull-down binding assay vs. the original molecule 185-H3-001 (FIG. 7). These experiments showed that a reduction of the seven terminal nucleotides (5′-end: GCUGGGC; 3′-end: GCCCAGC) of Type C C5a binding nucleic acid 185-H3-001 to five nucleotides could be only successfully done without reduction of binding affinity in the case of one pair of five terminal nucleotides (5′-end: GGGGC, 3′-end: GCCCC; 185-H3-014; pull-down assay see FIG. 16). However, the truncation to four terminal nucleotides (5′ end: GGGC; 3′ end: GCCC; 185-H3-003) or (5′ end: GGGA; 3′ end: UCCC; 185-B4-003) led to reduced binding affinity to C5a (FIG. 7).

However, combining the 5′- and 3′-terminal stretches of all tested Type C C5a binding nucleic acids the generic formula for the 5′-terminal stretch of Type C C5a binding nucleic acids is 5′ X₁X₂X₃BVGX₄M 3′ (Type C Formula-3-5′) and the generic formula for the 3′-terminal stretch Type C C5a binding nucleic acids is 5′ NX₅YBNX₆X₇X₈ 3′ (Type C Formula-3-3′), whereby X₁ is G or absent, X₂ is C or absent, X₃ is B or absent, X₄ is G, X₅ is C, X₆ is V or absent, X₇ is G or absent, X₈ is C or absent,

or
X₁ is G or absent, X₂ is C or absent, X₃ is B or absent, X₄ is absent, X₅ is absent, X₆ is V or absent, X₇ is G or absent, X₈ is C or absent,
whereby preferably X₁ is G, X₂ is C, X₃ is B, X₄ is absent, X₅ is absent, X₆ is V, X₇ is G, X₈ is C.

The best binding affinities of Type C C5a binding nucleic acids comprising 5′- and 3′-terminal stretches with four nucleotides, are shown for Type B C5a binding nucleic acid 185-H3-014 (5′-end: GGGGC, 3′-end: GCCCC).

For the PEGylated derivatives of C5a binding nucleic acids 185-H3-001 and 185-H3-014, 185-H3-001-5′-PEG and 185-H3-014-5′-PEG, IC₅₀'s of approx. 3.2 nM and 1.5 nM were determined in the TAX assay (FIG. 17).

In order to achieve more potent derivatives of Type C C5a binding nucleic acid 185-H3-14 new sequences of the 5′/3′-terminal stretches and the central stretch of the Type C C5a binding nucleic acid 185-H3-14 were designed, the new derivatives synthesized as spiegelmers and tested for their binding affinity to human C5a by plasmon resonance measurement. As shown for Type C C5a binding nucleic acid 185-H3-018 and 185-H3-019 the rearrangement of the sequence of the terminal stretches (185-H3-018) as well as the 3′-end of the central stretch (185-H3-019) led to a slightly declined binding affinity in comparison to Type C C5a binding nucleic acid 185-H3-14 (FIG. 26, 30A).

### 1.4 Type D C5a Binding Nucleic Acids

As depicted in FIG. 8 all sequences of C5a binding nucleic acids of Type D comprise one central sequence stretch or box defining a potential C5a binding motif which is flanked by 5′- and 3′-terminal stretches that can hybridize to each other. However, such hybridization is not necessarily given in the molecule.

It is within the present invention that—with regard to Type D C5a binding nucleic acids—the terms ‘5′-terminal stretch’ and ‘first stretch’, ‘central sequence’ and ‘second stretch’, and ‘3′-terminal stretch’ and “third stretch’, respectively are used herein in a synonymous manner if not indicated to the contrary.

The nucleic acids were characterized on the aptamer level using direct and competitive pull-down binding assays with biotinylated human D-05a in order to rank them with respect to their binding behaviour (Example 3). Selected sequences were synthesized as Spiegelmers (Example 2) and were tested using the natural configuration of human C5a (human L-05a) in a chemotaxis assay (Example 5).

The sequences of the defined boxes or stretches may be different between the C5a binding nucleic acids of Type D which influences the binding affinity to human C5a. Based on binding analysis of the different C5a binding nucleic acids summarized as Type D C5a binding nucleic acids, the central box and its nucleotide sequences as described in the following are individually and more preferably in their entirety essential for binding to human C5a:

The central box of all identified sequences of Type D C5a binding nucleic acids share the central sequence

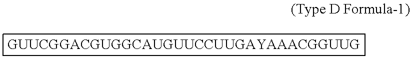

(FIG. 8). In order to determine the binding affinities of the different Type D C5a binding nucleic acids 182-E5, 182-05 and 182-A8 to human C5a they were tested on the aptamer level using direct and competitive pull-down binding assays with biotinylated human D-05a (Example 3). As reference the Type B C5a binding nucleic acid 179-A3-014 (IC₅₀=0.9 nM) was used. Type D C5a binding nucleic acids 182-E5 and 182-05 have better binding affinity to human C5a than Type B C5a binding nucleic acid 179-A3-014. Type D C5a binding nucleic acid 182-A8 (K_{D}=3.2 nM) showed in direct binding assay almost the same binding affinity as Type D C5a binding nucleic acids 182-E5 (K_{D}=2.4 nM, FIG. 18; IC50=1.2 nM, FIG. 19) and 182-05 (K_{D}=2.2 nM).

Seven nucleotides of the 5′-terminal stretch of Type D C5a binding nucleic acids can hybridize to the respective seven nucleotides of the 3′-terminal stretch to potentially form a terminal helix of seven base-pairing nucleotides. Although the seven base-pairing nucleotides are variable at several positions (see FIG. 8), the different nucleotides allow for hybridization of seven nucleotides of the 5′- and 3′-terminal stretches each.

In order to achieve more potent and shorter derivatives of Type D C5a binding nucleic acid 182-E5 new sequences of the terminal stretches and the central stretch of the Type D C5a binding nucleic acid 182-E5 were designed, the new derivatives synthesized as spiegelmers and tested for their binding affinity to human C5a by plasmon resonance measurement. As shown for the derivatives Type D C5a binding nucleic acid 182-E5 the rearrangement of the sequence of the 5′/3-terminal stretches and of the central stretch as well the reduction of the 5′/3′ terminal stretches led to a slightly declined binding affinity in comparison to Type D C5a binding nucleic acid 182-E5 (FIG. 27, 31A).

### 1.5 Further Nucleic Acids Binding to C5a

Additionally, 7 other C5a binding nucleic acids were identified which cannot be described by a combination of nucleotide sequence elements as has been shown for Types A, B, C, and D of C5a binding nucleic acids. These sequences are listed in FIG. 9.

The binding affinity of C5a binding nucleic acid 182-A5 to human C5a was determined by plasmon resonance measurement with a K_{D} of 0.3 to 0.4 nM. A truncated derivative of C5a binding nucleic acid 182-A5, C5a binding nucleic acid 182-A5-002, was measured with K_{D} of 0.3 nM. So that, the reduction of the terminal stretches of C5a binding nucleic acid 182-A5 are a promising tool to achieve shorter derivatives of C5a binding nucleic acid 182-A5 (FIG. 28, FIG. 31 B, FIG. 35). The C5a binding nucleic acid 182-A5 and 182-A2-002 comprise for the central stretch of nucleotides a nucleotide sequence of ‘GACAGGACCAAGGUAAGGGCGGACCGAAAAACCUAG’ and a first terminal stretch of nucleotides comprises four to five nucleotides and a second terminal stretch of nucleotides comprises four to five nucleotides, whereby the first terminal stretch of nucleotides comprises a nucleotide sequence of 5′ UGCUG 3′ and the second terminal stretch of nucleotides comprises a nucleotide sequence of 5′ CAGCA 3′.

It is to be understood that any of the sequences shown in FIGS. 1 through 9 are nucleic acids according to the present invention, including those truncated forms thereof but also including those extended forms thereof under the proviso, however, that the thus truncated and extended, respectively, nucleic acid molecules are still capable of binding to the target.

### EXAMPLE 2

### Synthesis and Derivatization of Aptamers and Spiegelmers

### Small Scale Synthesis

Aptamers (D-RNA nucleic acids) and Spiegelmers (L-RNA nucleic acids) were produced by solid-phase synthesis with an ABI 394 synthesizer (Applied Biosystems, Foster City, Calif., USA) using 2′TBDMS RNA phosphoramidite chemistry (Damha and Ogilvie, 1993). rA(N-Bz)-, rC(Ac)-, rG(N-ibu)-, and rU phosphoramidites in the D- and L-configuration were purchased from ChemGenes, Wilmington, Mass. Aptamers and Spiegelmers were purified by gel electrophoresis.

### Large Scale Synthesis Plus Modification

Spiegelmers were produced by solid-phase synthesis with an ÄktaPilot100 synthesizer (Amersham Biosciences; General Electric Healthcare, Freiburg) using 2′TBDMS RNA phosphoramidite chemistry (Damha and Ogilvie, 1993). L-rA(N-Bz)-, L-rC(Ac)-, L-rG(N-ibu)-, and L-rU phosphoramidites were purchased from ChemGenes, Wilmington, Mass. The 5′-amino-modifier was purchased from American International Chemicals Inc. (Framingham, Mass., USA). Synthesis of the unmodified or 5′-Amino-modified Spiegelmer was started on L-riboG, L-riboC, L-riboA or L-riboU modified CPG pore size 1000 Å (Link Technology, Glasgow, UK. For coupling (15 min per cycle), 0.3 M benzylthiotetrazole (CMS-Chemicals, Abingdon, UK) in acetonitrile, and 3.5 equivalents of the respective 0.1 M phosphoramidite solution in acetonitrile was used. An oxidation-capping cycle was used. Further standard solvents and reagents for oligonucleotide synthesis were purchased from Biosolve (Valkenswaard, NL). The Spiegelmer was synthesized DMT-ON; after deprotection, it was purified via preparative RP-HPLC (Wincott et al., 1995) using Source 15RPC medium (Amersham). The 5′DMT-group was removed with 80% acetic acid (30 min at RT). Subsequently, aqueous 2 M NaOAc solution was added and the Spiegelmer was desalted by tangential-flow filtration using a 5 K regenerated cellulose membrane (Millipore, Bedford, Mass.).

### PEGylation of Spiegelmers

In order to prolong the Spiegelmer's plasma residence time in vivo, Spiegelmers was covalently coupled to a 40 kDa polyethylene glycol (PEG) moiety at 5′-end.

### 5′-PEGylation of Spiegelmers

For PEGylation (for technical details of the method for PEGylation see European patent application EP 1 306 382), the purified 5′-amino modified Spiegelmer was dissolved in a mixture of H₂O (2.5 ml), DMF (5 ml), and buffer A (5 ml; prepared by mixing citric acid.H₂O [7 g], boric acid [3.54 g], phosphoric acid [2.26 ml], and 1 M NaOH [343 ml] and adding water to a final volume of 1 l; pH=8.4 was adjusted with 1 M HCl).

The pH of the Spiegelmer solution was brought to 8.4 with 1 M NaOH. Then, 40 kDa PEG-NHS ester (Jenkem Technology, Allen, Tex., USA) was added at 37° C. every 30 min in six portions of 0.25 equivalents until a maximal yield of 75 to 85% was reached. The pH of the reaction mixture was kept at 8-8.5 with 1 M NaOH during addition of the PEG-NHS ester.

The reaction mixture was blended with 4 ml urea solution (8 M), and 4 ml buffer B (0.1 M triethylammonium acetate in H₂O) and heated to 95° C. for 15 min. The PEGylated Spiegelmer was then purified by RP-HPLC with Source 15RPC medium (Amersham), using an acetonitrile gradient (buffer B; buffer C, 0.1 M triethylammonium acetate in acetonitrile). Excess PEG eluted at 5% buffer C, PEGylated Spiegelmer at 10-15% buffer C. Product fractions with a purity of >95% (as assessed by HPLC) were combined and mixed with 40 ml 3 M NaOAC. The PEGylated Spiegelmer was desalted by tangential-flow filtration (5 K regenerated cellulose membrane, Millipore, Bedford Mass.).

### EXAMPLE 3

### Determination of Binding Constants to C5a (Pull-Down Assay)

### Direct Pull-Down Assay

The affinity of C5a binding nucleic acids were measured as aptamers (D-RNA nucleic acids) to biotinylated human D-05a (SEQ.ID. 2) in a pull down assay format at 37° C. Aptamers were 5′-phosphate labeled by T4 polynucleotide kinase (Invitrogen, Karlsruhe, Germany) using [γ-³²P]-labeled ATP (Hartmann Analytic, Braunschweig, Germany). The specific radioactivity of labeled aptamers was 200,000-800,000 cpm/pmol. Aptamers were incubated after de- and renaturation at 20 pM concentration at 37° C. in selection buffer (20 mM Tris-HCl pH 7.4; 137 mM NaCl; 5 mM KCl; 1 mM MgCl₂; 1 mM CaCl₂; 0.1% [w/vol] Tween-20) together with varying amounts of biotinylated human D-05a for 4-12 hours in order to reach equilibrium at low concentrations. Selection buffer was supplemented with 10 μg/ml human serum albumin (Sigma-Aldrich, Steinheim, Germany), and 10 μg/ml yeast RNA (Ambion, Austin, USA) in order to prevent adsorption of binding partners with surfaces of used plasticware or the immobilization matrix. The concentration range of biotinylated human D-05a was set from 7 pM to 200 nM; total reaction volume was 1 ml. Biotinylated human D-05a and complexes of aptamer and biotinylated human D-05a were immobilized on 4 μl Streptavidin Ultralink Plus particles (Pierce Biotechnology, Rockford, USA) which had been preequilibrated with selection buffer and resuspended in a total volume of 12 μl. Particles were kept in suspension for 30 min at the respective temperature in a thermomixer. Immobilized radioactivity was quantitated in a scintillation counter after detaching the supernatant and appropriate washing. The percentage of binding was plotted against the concentration of biotinylated human D-05a and dissociation constants were obtained by using software algorithms (GRAFIT; Erithacus Software; Surrey U.K.) assuming a 1:1 stoichiometry.

### Competitive Pull-Down Assay

In order to compare different biotinylated human D-05a binding aptamers, a competitive ranking assay was performed. For this purpose the most affine aptamer available was radioactively labeled (see above) and served as reference. After de- and renaturation it was incubated at 37° C. with biotinylated human D-05a in 1 ml selection buffer at conditions that resulted in around 5-10% binding to the biotinylated human D-05a after immobilization and washing on NeutrAvidin agarose or Streptavidin Ultralink Plus (both from Pierce) without competition. An excess of de- and renatured non-labeled D-RNA aptamer variants was added to different concentrations (e.g. 2, 10, and 50 nM) with the labeled reference aptamer to parallel binding reactions. The aptamers to be tested competed with the reference aptamer for target binding, thus decreasing the binding signal in dependence of their binding characteristics. The aptamer that was found most active in this assay could then serve as a new reference for comparative analysis of further aptamer variants.

### EXAMPLE 4

### Determination of Inhibitory Concentration in a Ca⁺⁺-Release Assay

U937 cells (DSMZ, Braunschweig, Germany) were cultivated at 37° C. and 5% CO₂ in RPMI 1640 medium with GlutaMAX (Invitrogen, Karlsruhe, Germany) which contained in addition 10% fetal calf serum, 50 units/ml penicillin and 50 μg/ml streptomycin. Two days before an experiment, cells are seeded in a new flask with a density of 0.2×10⁶/ml (6×10⁶/30 ml) in standard medium to which dibutyryl-cAMP is added to result in a final concentration of 1 mM.

The Spiegelmers were incubated together with recombinant human C5a (SEQ.ID. 1) in Hanks balanced salt solution (HBSS), containing 1 mg/ml bovine serum albumin, 5 mM probenecid and 20 mM HEPES (HBSS⁺) for 15 to 60 min at 37° C. in a 0.2 ml low profile 96-tube plate (“stimulation solution”).

For loading with the calcium indicator dye, cells were centrifuged at 300×g for 5 min, resuspended in 4 ml indicator dye solution (10 μM fluo-4 [Molecular Probes], 0.08% pluronic 127 [Molecular Probes] in HBSS+) and incubated for 60 min at 37° C. Thereafter, 11 ml HBSS+ were added and the cells were centrifuged as above, washed once with 15 ml HBSS+ and then resuspended in HBSS+ to give a cell density of 1.1×10⁶/ml. 90 μl of this cell suspension were added to each well of a black 96-well plate.

Measurement of fluorescence signals was done at an excitation wavelength of 485 nm and an emission wavelength of 520 nm in a Fluostar Optima multidetection plate reader (BMG, Offenburg, Germany). For parallel measurement of several samples, wells of one (perpendicular) row of a 96-well plate were recorded together. First three readings with a time lag of 4 sec were done for determination of the base line. Then the recording was interrupted and the plate was moved from the instrument. Using a multi-channel pipette, 10 μl of the stimulation solution was added to the wells, then the plate was moved into the instrument again and the measurement was continued. In total, 20 recordings with time intervals of 4 seconds were performed.

For each well the difference between maximal fluorescence and base line value was determined and plotted against C5a concentration or, in the experiments on the inhibition of calcium release by Spiegelmers, against concentration of Spiegelmer.

### Determination of Half-Maximal Effective Concentration (EC₅₀) for Human C5a

After stimulation of U937 cells with various C5a concentrations and plotting the difference between the maximal and the baseline signals, a dose-response curve for human C5a was obtained, indicating a half effective concentration (EC₅₀) of about 1 nM. This concentration was used for the further experiments on inhibition of Ca⁺⁺-release by Spiegelmers.

### EXAMPLE 5

### Determination of Inhibitory Concentration in a Chemotaxis Assay

U937 cells grown and differentiated as described above were centrifuged, washed once in HBH (HBSS, containing 1 mg/ml bovine serum albumin and 20 mM HEPES) and resuspended at 3×10⁶ cells/ml. 100 μl of this suspension were added to Transwell inserts with 5 μm pores (Costar Corning, #3421; NY, USA). In the lower compartments recombinant human C5a (SEQ.ID. 1) was preincubated together with Spiegelmers in various concentrations in 600 μl HBH at 37° C. for 20 to 30 min prior to addition of cells. Cells were allowed to migrate at 37° C. for 3 hours. Thereafter the inserts were removed and 60 μl of 440 μM resazurin (Sigma, Deisenhofen, Germany) in phosphate buffered saline was added to the lower compartments. After incubation at 37° C. for 2.5 hours, fluorescence was measured at an excitation wavelength of 544 nm and an emission wavelength of 590 nm in a Fluostar Optima multidetection plate reader (BMG, Offenburg, Germany).

Alternatively U937 cells prepared as described above were resuspended at 1.33×10⁶ cells/ml. 75 μl of this suspension were added to the upper compartments of a 96 well Corning Transwell plate with 5 μm pores (Costar Corning, #3388; NY, USA). In the lower compartments recombinant human C5a (SEQ.ID. 1) was preincubated together with Spiegelmers in various concentrations in 235 μl HBH at 37° C. for 20 to 30 min prior to addition of cells. Cells were allowed to migrate at 37° C. for 3 hours. Thereafter the insert plate (upper compartments) was removed and 30 μl of 440 μM resazurin (Sigma, Deisenhofen, Germany) in phosphate buffered saline was added to the lower compartments. After incubation at 37° C. for 2.5 hours, fluorescence was measured as described above.

Fluorescence values are corrected for background fluorescence (no cells in well). Then the difference between experimental conditions with and without C5a is calculated. These results can be depicted in a histogram. Alternatively or in addition to this, the value for the sample without Spiegelmer (C5a only) is set 100% and the values for the samples with Spiegelmer are calculated as percent of this. For a dose-response curve the percent values are plotted against Spiegelmer concentration and the IC₅₀-value (concentration of Spiegelmer at which 50% of the activity without Spiegelmer is present) is determined graphically from the resulting curve.

### Determination of Half-Maximal Effective Concentration (EC₅₀) for Human C5a

After 3 hours migration of U937 cells towards various human C5a concentrations, a dose-response curve for human C5a was obtained, indicating a maximal effective concentration of about 1 nM and reduced activation at higher concentrations. For the further experiments on inhibition of chemotaxis by Spiegelmers a C5a concentration of 0.1 nM was used.

### EXAMPLE 6

### Determination of Binding Constants to C5 (Filter Binding Assay)

The affinity of Spiegelmers to complement component 5 from human blood (human L-05; Sigma Aldrich, Taufkirchen, Germany (Cat No. C3160); consisting of the human C5 alpha chain see SEQ.ID. 171, human C5 beta chain see SEQ.ID. 172) was measured in a filter binding assay format at 37° C. Spiegelmers were synthesized with two additional D-guanosine moieties at the 5′ end allowing for labeling by T4 polynucleotide kinase with [γ-³²P]-ATP. The specific radioactivity of labeled Spiegelmers was 300,000-500,000 cpm/pmol. Spiegelmers were incubated after heat de- and renaturation at 30 pM concentration at 37° C. in binding buffer (20 mM Tris-HCl, pH 7.4; 150 mM NaCl; 5 mM KCl; 1 mM MgCl₂; 1 mM CaCl₂; 0.001% [w/vol] Tween-20) together with varying amounts of C5 for 4-6 hours. Binding buffer was supplemented with 10 μg/ml human serum albumin in order to prevent adsorption of binding partners with surfaces of the plasticware used. The concentration range of C5 was set from 7 μM to 100 nM; the total reaction volume was 0.4 ml. Nitrocellulose (NC) filters with 0.22 μm pore size and 10 mm diameter (Millipore, Schwalbach, Germany) were soaked for 5 min in H₂O and placed on a vacuum manifold (Mallinckrodt Baker, Germany). Before transfer of the binding reactions to the NC filters a vacuum corresponding to −5 inches of Hg was applied on the filter via the vacuum manifold. The binding reactions passed through the filters and C5 was retained on the filter—together with labeled Spiegelmer, if the latter was in complex with C5. The percentage of bound Spiegelmer was measured in a scintillation counter after appropriate washing with buffer without BSA. The percentage of filter-bound Spiegelmer was plotted against the concentration of C5 and dissociation constants were obtained by using the software (GRAFIT; Erithacus Software; Surrey U.K.) assuming a 1:1 stoichiometry.

The Type A C5a binding nucleic acids 172-D7-000 (SEQ. ID. 3) and 172-D7-013 (SEQ. ID. 14), the Type B C5a binding nucleic acids 179-A3-014 (SEQ.ID. 36) and 179-A3-015 (SEQ.ID. 38), the Type C C5a binding nucleic acids 185-H3-001 (SEQ.ID. 49), 185-H3-002 (SEQ.ID. 63), 185-H3-014 (SEQ.ID. 65) and 185-H3-003 (SEQ.ID. 67) and Type D C5a binding nucleic acids 182-E5 (SEQ. ID. 69) and 182-05 (SEQ. ID. 70) were synthesized as spiegelmers with two D-guanosine moieties at the 5′ end allowing for labelling by T4 polynucleotide kinase with [γ-³²P]-ATP. All such modified spiegelmers (SEQ.ID's. 157-167) showed binding affinity to human C5 comparable to their respective binding behaviour to human C5a (Individual binding affinities of the corresponding aptamer sequences to synthetic human D-05a see FIGS. 1-8). The data for C5a binding nucleic acids 172-D7-013, 179-A3-014 and 185-H3-014 are shown in FIG. 20.

Besides the fact that the entire C5 molecule is bound by these molecules, this experiment shows that biological C5 from human serum and therefore with its natural glycosylation is also bound by the Spiegelmers described here.

### EXAMPLE 7

### Proof of Concept: Activity of a Selected C5a Spiegelmer In Vivo

To test the ability of Spiegelmer 185-H3-014-5′-PEG to block C5a action in vivo, the known property of human C5a to induce neutropenia in gerbils (Sumichika et al., 2002) was utilized as a model for septic shock.

### Method

Anesthetized female Mongolian gerbils (Charles River, Germany, 7-8 weeks old, n=7 per group) received a single i.v. injection of anti-05a Spiegelmer 185-H3-014-5′-PEG (2 mg/kg or 10 mg/kg oligonucleotide in 5% glucose) or vehicle (5% glucose). A PEGylated Spiegelmer of the same base composition but the reverse sequence, that does not bind to C5a was used to differentiate C5a-binding related effects from unspecific interference with the model by Spiegelmers in general. The reverse Spiegelmer 185-H3-014-REVERSE-5′-PEG was also dosed at 2 mg/kg or 10 mg/kg oligonucleotide in 5% glucose in additional control groups. After 8 to 9 min, blood was collected via intracardiac puncture from the animals. This was followed by an i.v. bolus injection of 100 μg/kg human recombinant C5a (Sigma, Deisenhofen, Germany Cat No. #C5788). Blood was subsequently collected 1, 3 and 5 min after the C5a injection. The samples were immediately transferred into tubes containing EDTA as anticoagulant.

Blood smears were prepared from the blood samples and stained with May Grünwald-Giemsa staining. 100 white blood cells on each blood smear were counted and differential cell numbers determined for neutrophils, eosinophils, basophils, lymphocytes and monocytes. For each animal the percentage of neutrophils was determined for the time points 1 and 5 min and expressed as percentage of the neutrophil count for time point 0.

### Results

Injection of C5a leads to a rapid reduction of neutrophils in the blood: one min after injection, the neutrophil count was reduced to ca. 30% of the value before injection. Three minutes later, the value is already higher again (ca. 55%) and rises to ca. 70% 5 min post injection of C5a, which indicates that the process is reversible. These in vivo findings are quite in line with the data published by Sumichika et al., who reported a reduction to ca. 20% in a very similar experiment. This decrease in neutrophil number (neutropenia) is significantly attenuated by application of Spiegelmer 185-H3-014-5′-PEG (10 mg/kg oligonucleotide) prior to injection of C5a as depicted in FIG. 21 at 1 min and 3 min post C5a application. The dose group of 2 mg/kg did not lead to an inhibition of neutropenia. This may be due to the fast kinetics of the C5a-mediated effect. The reverse Spiegelmer 185-H3-014-REVERSE-5′-PEG did not lead to a reduction of the human recombinant C5a-induced neutropenia in both tested concentrations.

### EXAMPLE 8

### Binding of C5a Binding Spiegelmers to Rhesus Monkey C5a Method

The sequence of rhesus monkey (Macaca mulatta) C5a was deduced from the predicted sequence for complement component 5 (accession XM_{—}001095750). The sequence presumably coding for C5a was amplified from rhesus monkey total liver RNA (BioCat) by RT-PCR using the primers 5′-ATGCTACAAGAGAAGATAGAAG (C5a-Primer-I) and 5′-CTAGCATGCTTACCTTCCCAATTGC(C5a-Primer-II) and cloned into the pQE30Xa vector (Qiagen, Hilden, Germany).

The resulting protein (Pubmed accession No. XP_{—}001095750, SEQ.ID. 186) is 85% (63 of 74 amino acids) identical to human C5a (SEQ.ID. 1).

To compare the C5a-sequence from rhesus monkey with that from cynomolgus monkey (Macaca fascicularis) a RT-PCR with cynomolgus monkey total liver RNA using the primers 5′-TGGGTGGCATTAACAGCAGTG (CynoC5a_up) and 5′-TCCCAGGTAGTTACAGAATCAGGT (CynoC5a_low) was performed followed by sequencing of the PCR product. Comparison of the sequences showed that the DNA sequences coding for C5a were identical for M. mulatta and M. fascicularis.

The His6-tagged monkey C5a was expressed in E. coli BL21 and purified with nickel affinity chromatography (HIS-Select, Sigma, Deisenhofen, Germany or HisTrap FF crude, GE Healthcare, Freiburg, Germany) in 100 mM phosphate buffers containing 8 M urea. The protein was eluted with 250 mM imidazole and stored at −20° C. Prior to use in chemotaxis assays (see example 5) the protein was diluted (1:10) in renaturation buffer (50 mM Tris/HCl, pH 8.0, 0.005% Tween 20, 2 mM reduced glutathione, 0.2 mM oxidized glutathione) and incubated for at least 10 min at room temperature before further dilution in HBH.

Alternatively, the protein was renatured on the column by applying a gradient (30 column volumes) from the 8M urea buffer to a reducing buffer (100 mM Na-phosphate, pH 8.0, 50 mM NaCl, 10 mM reduced glutathione, 10 mM imidazole) and eluted with 250 mM imidazole in this buffer. The buffer was then changed to 20 mM MES pH 6.5, 50 mM NaCl by chromatography on a NAP™-25 column (GE Healthcare, Freiburg, Germany). After determination of protein concentration with the BCA method, the protein was stored at −20° C.

### Result

Chemotaxis assays were performed as described in example 5 using the purified monkey C5a (His6-macC5a) or recombinant human C5a. The final concentration of His6-macC5a was approximately 0.8 nM according to protein determination and gave a chemotactic response of U937 cells similar to 0.1 nM human C5a. The tested Spiegelmers of Type C C5a binding nucleic acids 185-H3-014-5′PEG and 185-H3-001 as well as the Spiegelmer of Type B C5a binding nucleic acid 179-A3-014-5′PEG were applied at 100 nM.

Whereas Spiegelmers of Type C C5a binding nucleic acids 185-H3-014-5′PEG and 185-H3-001 could not inhibit the action of 0.8 nM His6macC5a, 100 nM Spiegelmer Type B C5a binding nucleic acid 179-A3-014-5′PEG completely blocked the chemotaxis of U937 cells induced by 0.8 nM His6macC5a (FIG. 22).

In another experiment it could be shown that the Spiegelmers of Type B C5a binding nucleic acids 182-D5 and 182-E6 as well as C5a binding nucleic acid 182-A5 applied at 10 nM efficiently blocked the chemotaxis of U937 cells induced by 1 and 3 nM His6macC5a. The Spiegelmer of Type D C5a binding nucleic acid 182-E5 showed only weak inhibitory effect on macaque C5a.

### EXAMPLE 9

### Binding Analysis by Surface Plasmon Resonance Measurement

The Biacore 2000 instrument (Biacore AB, Uppsala, Sweden) was used to analyze binding of spiegelmers to human C5a, macaque C5a and to human C5 (purified from human plasma).

The instrument was set to a enduring temperature of 37° C. Before the start of each experiment the Biacore was cleaned using the DESORB method according to the manufacturer's instructions. After docking a maintenance chip, the instrument was consecutively primed with DESORB solution 1 (0.5% sodium dodecyl sulphate, SDS), DESORB solution 2 (50 mM glycine, pH 9.5) and finally degassed MilliQ water. Subsequently the SANATIZE method was run with 0.1 M NaOCl and the system was primed afterwards with MilliQ water.

Human C5a (Sigma, Cat. No, C5788), human C5 (Sigma, Cat. No. C3160) and macaque C5a (His6x-05a from macaque monkey was cloned, recombinant expressed and purified as described, see Example 8) were dissolved in water at a concentration of 100 μg ml in prelubricated vials and stored at −70° C. until use.

After docking a sensor chip with a carboxymethylated dextran matrix (Sensor Chip CM5, GE, BR-1000-14), the Biacore instrument was primed with MilliQ water followed by HBS-EP buffer (0.01 M HEPES buffer [pH 7.4], 0.15 M NaCl, with 0.005% Surfactant P20; GE, BR-1001-88) and equilibrated until a stable baseline was observed. The flow cells (FCs) were immobilized beginning from flow cell 4 to flow cell 1 to avoid carry-over of peptides to other flow cells.

C5a peptides were either immobilized by amine or thiol surface coupling procedure. For amine coupling of C5a peptide or C5 protein 100 μl of a 1:1 mixture of 0.4M EDC (1-ethyl-3-(3-dimethylaminopropyl)carbodiimide in H₂O; GE, BR-1000-50) and 0.1M NHS(N-hydroxysuccinimide in H₂O; GE, BR-1000-50) were injected using the QUICKINJECT command at a flow of 10 μl/min. Activation of the flow cell was monitored by an increase in RU after NHS EDC injection (typically 500-600 RU for CM5 chips). C5a was dissolved in 10 mM sodiumactetat, pH 5.5 to a concentration of 1-10 μg/ml and subsequently injected using the MANUALINJECT command at a flow of 10 μl min. The maximal observed amount of covalently immobilized C5a was about 4000 RU; the immobilization level for kinetic evaluation was 1500-2000 RU. The flow cells were blocked with an injection 70 μl of 1 M ethanolamine hydrochloride (GE, BR-1000-50) at a flow of 10 μl min.

For surface thiol coupling of C5a or C5 the ligand was conjugated to a PDEA linker and covalently coupled to cystamine on the sensor chip surface using a thiol coupling kit (Biacore, Ca. No. Br-1005-57). 0.5 mg ml C5a or C5 were dissolved in 0.5 ml 0.1M MES buffer pH 5.0. 0.25 ml of 15 mg ml PDEA-linker and 25 μl EDC were added and incubated for 10 minutes at 25° C. The excess of reagents was removed by adding 500 μl of the sample volume to a pre-equilibrated NAP-5 column (Amersham Biosciences) and the C5a or C5, coupled to PDEA, was eluted from the column with 1 ml of MilliQ water. For covalent coupling of PDEA-05a C5 to a CM5 sensor chip 100 μl of a 1:1 mixture of 0.4M EDC (1-ethyl-3-(3-dimethylaminopropyl)carbodiimide in H₂O; GE, BR-1000-50) and 0.1M NHS (N-hydroxysuccinimide in H₂O; GE, BR-1000-50), followed by 100 μl of 40 mM cystamine dihydrochlorid and subsequently 100 μl of 0.1 M1,4-Dithioerythriol (DTE) were injected using the QUICKINJECT command at a flow of 10 μl min. PDEA-05a or PDEA-05 was injected using the MANUALINJECT command until 1500-3000 RU were immobilized on the sensor chip surface. The flow cell was blocked by a 40 μl injection of PDEA-NaCl. Subsequently the flow cell was washed with 1 M NaCl (Ambion, Cat. No. AM9759) to avoid carry over of PDEA-05a due to unspecific interaction with the Biacore tubing and other surfaces. FC1 served as blocked control flow cell.

The sensor chip was primed twice with degassed running buffer (20 mM Tris pH 7.4; 150 mM NaCl; 5 mM KCl, 1 mM MgCl₂, 1 mM CaCl₂ and 0.1% Tween20) and equilibrated at 50 μl min until the baseline appeared stable.

Typically for analytical purpose, C5a binding spiegelmers were diluted in water to a stock concentration of 100 μM (quantification by UV measurement), heated up to 95° C. for 30 seconds in a water bath or thermo mixer and snap cooled on ice to assure a homogenous dissolved solution.

Kinetic parameters and dissociation constants were evaluated by a series of spiegelmer injections at concentrations of 1000, 500, 250, 125, 62.5, 31.25, 15.63, 7.8, 3.9 and 0 nM diluted in running buffer. In all experiments, the analysis was performed at 37° C. using the Kinject command at a flow of 30 μl min. The assay was double referenced, whereas FC1 served as (blocked) surface control (bulk contribution of each spiegelmer concentration) and a series of buffer injections without analyte determined the bulk contribution of the buffer itself. Data analysis and calculation of dissociation constants (K_{D}) was performed with the BIAevaluation 3.0 software (BIACORE AB, Uppsala, Sweden) using the Langmuir 1:1 stoichiometric fitting algorithm.

### EXAMPLE 10

### C5a Binding Nucleic Acids do not Interfere with Complement-Dependent Hemolysis

The ultimate product of the complement cascade is the membrane attack complex (MAC), a pore consisting of C5b-9. MAC is believed to insert into the cytoplasmic membranes of pathogens and kill them by induction of cytoplasmic leakage.

The C5a binding nucleic acids (spiegelmers) presented here have been shown to recognize C5a in the context of C5 (see Example 6, FIG. 20 and FIG. 23-28)). Therefore it was investigated whether C5 cleavage to the anaphylatoxin C5a and C5b, which is part of the MAC is inhibited by these the spiegelmers. This was achieved by using a complement-dependent sheep erythrocyte hemolysis test.

### Methods

Reconstituted human lyophilized serum (‘normal control’ reagent from “CHSO Eq Enzyme Immunoassay Kit” (QUIDEL/USA) was pre-incubated with spiegelmers in the range of 2 to 6000 nM spiegelmer in 96-well plates (Nunc-Immuno™ Plate, MaxiSorp Surface™). As a control the C5-binding aptamer C5C6 (Biesecker et al. 1999) (synthesized in house) which inhibits C5 cleavage was used in the same concentration range. After 1 hour incubation at 37° C. the hemolytic system was added to the pre-incubated serum complement inhibitor mixture. The hemolytic system contains sheep erythrocytes, which are opsonized by goat anti-sheep erythrocyte antibodies, known as hemolytic system (Institut Virion/Serion GmbH). Hemolysis was determined 30 min later by a colorimetric measurement after spinning down intact cells. The higher the degree of hemolysis the higher the absorption at 405 nm (measured in a Fluo Star).

### Results

The aptamer C5C6 inhibited complement-dependent lysis of the hemolytic system with an IC₅₀ of approximately 1 μM. The spiegelmers tested, namely C5a binding nucleic acids 182-E5, 182-E5-5′PEG, 182-D5-001, 182-A5-001, 182-E6-001 and the non-05-binding Spiegelmer ‘PoC’ only weakly inhibited hemolysis cleavage. Since PoC shows the same trend as the C5a-binding spiegelmers, the weak effect seen is probably due to unspecific interactions of the spiegelmers with some part of the complement cascade.

### Discussion

The C5a binding spiegelmers tested were shown not to inhibit MAC formation and are therefore selective antagonists of C5a only. If used as a medicine, this may be advantageous in many diseases, since the formation of the MAC that is beneficial in pathogen defense is not compromised in their presence.

### REFERENCES

The complete bibliographic data of the documents recited herein the disclosure of which is incorporated by reference is, if not indicated to the contrary, as follows.
Altschul S F, Gish W, Miller W, Myers E W, Lipman D J (1990), Basic local alignment search tool. J Mol. Biol. 215(3):403-10.
Altschul S F, Madden T L, Schaffer A A, Zhang J, Zhang Z, Miller W, Lipman D J (1997). Gapped BLAST and PSI-BLAST: a new generation of protein database search programs. Nucleic Acids Res. 25(17):3389-402.
Arumugam T V, Shiels I A, Strachan A J, Abbenante G, Fairlie D P, Taylor S M (2003) A small molecule C5a receptor antagonist protects kidneys from ischemia/reperfusion injury in rats. Kidney Int 63(1): 134-142
Arumugam T V, Woodruff T M, Stocks S Z, Proctor L M, Pollitt S, Shiels I A, Reid R C, Fairlie D P, Taylor S M (2004) Protective effect of a human C5a receptor antagonist against hepatic ischaemia-reperfusion injury in rats. J Hepatol 40(6): 934-941
Aurup H et al. (1994). Nucleic Acids Res 22:20
Bergh K, Iversen O J, Lysvand H. 1993. Surprisingly high levels of anaphylatoxin C5a des Arg are extractable from psoriatic scales. Arch Dermatol Res 285(3):131-134.
Biesecker G, Dihel L, Enney K, Bendele R A (1999) Derivation of RNA aptamer inhibitors of human complement C5. Immunopharmacology 42(1-3): 219-230.
Bonifati D M, Kishore U. 2007 Role of complement in neurodegeneration and neuroinflammation. Mol Immunol 44(5): 999-1010.
Chen M, Daha M R, Kallenberg C G (2010) The complement system in systemic autoimmune disease. J Autoimmun 34(3): J276-286
Copland D A, Hussain K, Baalasubramanian S. Hughes T R, Morgan B P, Xu H, Dick A D, Nicholson L B (2010) Systemic and local anti-05 therapy reduces the disease severity in experimental autoimmune uveoretinitis. Clin Exp Immunol 159(3): 303-314
Cummins L L et al. (1995). Nucleic Acids Res 23:2019
Damha M J and Ogilvie K K, Methods in Molecular Biology, Vol. 20 Protocols for oligonucleotides and analogs, ed. S. Agrawal, p. 81-114, Humana Press Inc. 1993
Durand M, Chevrie K, Chassignol M, Thuong N T, and Maurizot J C (1990), Circular dichroism studies of an oligodeoxyribonucleotide containing a hairpin loop made of a hexaethylene glycol chain: conformation and stability. Nucleic. Acids Res 18: 6353-6359.
Eaton B E et al. (1995). Chem Biol 2:633
Eaton B E, Gold L, Hicke B J, Janjic N, Jucker F M, Sebosta D P, Tarasow T M, Willis M C, Zichi D A (1997). Bioorg Med Chem 5:1087
Farkas I, Baranyi L, Liposits Z S, Yamamoto T, Okada H (1998) Complement C5a anaphylatoxin fragment causes apoptosis in TGW neuroblastoma cells. Neuroscience 86(3): 903-911
Fernandez H N, Hugli T E (1978) Primary structural analysis of the polypeptide portion of human C5a anaphylatoxin. Polypeptide sequence determination and assignment of the oligosaccharide attachment site in C5a. J Biol Chem 253(19): 6955-6964
Fernandez H N, Hugli T E. 1978. Primary structural analysis of the polypeptide portion of human C5a anaphylatoxin. Polypeptide sequence determination and assignment of the oligosaccharide attachment site in C5a. J Biol Chem 253(19):6955-6964.
Fonseca M I, Ager R R, Chu S H, Yazan O, Sanderson S D, LaFerla F M, Taylor S M, Woodruff T M, Tenner A J (2009) Treatment with a C5aR antagonist decreases pathology and enhances behavioral performance in murine models of Alzheimer's disease. J Immunol 183(2): 1375-1383
Girardi G (2008) Guilty as charged: all available evidence implicates complement's role in fetal demise. Am J Reprod Immunol 59(3): 183-192
Green L S et al. (1995). Chem Biol 2:683
Heller T, Hennecke M, Baumann U, Gessner J E, zu Vilsendorf A M, Baensch M, Boulay F, Kola A, Klos A, Bautsch W, Kohl J. 1999. Selection of a C5a receptor antagonist from phage libraries attenuating the inflammatory response in immune complex disease and ischemia/reperfusion injury. J Immunol 163(2):985-994.
Hillmen P, Muus P, Duhrsen U, Risitano A M, Schubert J, Luzzatto L, Schrezenmeier H, Szer J, Brodsky R A, Hill A, Socie G, Bessler M, Rollins S A, Bell L, Rother R P, Young N S (2007). Effect of the complement inhibitor eculizumab on thromboembolism in patients with paroxysmal nocturnal hemoglobinuria. Blood, August 2007, PMID: 17702897
Huber-Lang M S, Sarma J V, McGuire S R, Lu K T, Guo R F, Padgaonkar V A, Younkin E M, Laudes I J, Riedemann N C, Younger J G, Ward P A. 2001. Protective effects of anti-05a peptide antibodies in experimental sepsis. Faseb J 15(3):568-570.
Jacob A, Hack B, Bai T, Brorson J R, Quigg R J, Alexander J J (2010a) Inhibition of C5a receptor alleviates experimental CNS lupus. J Neuroimmunol
Jacob A, Hack B, Chiang E, Garcia J G, Quigg R J, Alexander J J (2010b) C5a alters blood-brain barrier integrity in experimental lupus. FASEB J
Jang J H, Clark J D, Li X, Yorek M S, Usachev Y M, Brennan T J Nociceptive sensitization by complement C5a and C3a in mouse. Pain 148(2): 343-352
Kawasaki A M et al. (1993). J Med Chem 36:831
Kirschfink M. 1997. Controlling the complement system in inflammation. Immunopharmacology 38(1-2):51-62.
Klos A, Tenner A J, Johswich K O, Ager R B, Reis E S, Kohl J (2009) The role of the anaphylatoxins in health and disease. Mol Immunol 46(14): 2753-2766
Kohl J. 2001. Anaphylatoxins and infectious and non-infectious inflammatory diseases. Mol Immunol 38(2-3):175-187.
Kusser W (2000). J Biotechnol 74:27-38
Lesnik E A et al. (1993). Biochemistry 32:7832
Lewis A G, Kohl G, Ma Q, Devarajan P, Kohl J. 2008 Pharmacological targeting of C5a receptors during organ preservation improves kidney graft survival. Clin Exp Immunol 153(1): 117-126.
Lewis F D, Liu X, Wu Y, Miller S E, Wasielewski M R, Letsinger R L, Sanishvili R, Joachimiak A, Tereshko V and Egli M (1999). Structure and photoinduced eletron transfer in exceptionally stable synthetic DNA hairpins with stilbenediether linkers. JACS 121:9905-9906.
Ma M Y, Reid L S, Climie S C, Lin W C, Kuperman R, Sumner-Smith M, Barnett R W (1993a). Design and synthesis of RNA miniduplexes via a synthetic linker approach. Biochemistry 32(7):1751-1758.
Makrides S C. 1998. Therapeutic inhibition of the complement system. Pharmacol Rev 50(1):59-87.
Manderson A P, Botto M, Walport M J. 2004. The role of complement in the development of systemic lupus erythematosus. Arum Rev Immunol 22: 431-456
Marc M M, Korosec P, Kosnik M, Kern I, Flezar M, Suskovic S, Sorli J (2004). Complement factors c3a, c4a, and c5a in chronic obstructive pulmonary disease and asthma. Am J Respir Cell Mol. Biol. 31(2):216-9.
Markiewski M M, DeAngelis R A, Benencia F, Ricklin-Lichtsteiner S K, Koutoulaki A, Gerard C, Coukos G, Lambris J D (2008). Modulation of the antitumor immune response by complement. Nat. Immunol. (11):1225-35
McGinnis S, Madden T L (2004). BLAST: at the core of a powerful and diverse set of sequence analysis tools. Nucleic Acids Res. 32(Web Server issue):W20-5.
Miller L E et al. (1993). J Physiol 469:213
Muller-Ladner U, Jones J L, Wetsel R A, Gay S, Raine C S, Barnum S R. 1996. Enhanced expression of chemotactic receptors in multiple sclerosis lesions. J Neurol Sci 144(1-2):135-141.
Needleman & Wunsch (1970), A general method applicable to the search for similarities in the amino acid sequence of two proteins. J Mol. Biol. 48(3):443-53.
Nozaki M, Raisler B J, Sakurai E, Sarma J V, Barnum S R, Lambris J D, Chen Y, Zhang K, Ambati B K, Baffi J Z, Ambati J. 2006. Drusen complement components C3a and C5a promote choroidal neovascularization. Proc Natl Acad Sci USA 103(7): 2328-2333
Ostrand-Rosenberg S (2008). Cancer and complement. Nat. Biotechnol. 26(12):1348-9.
Patel S N, Berghout J, Lovegrove F E, Ayi K, Conroy A, Serghides L, Min-oo G, Gowda D C, Sarma J V, Rittirsch D, Ward P A, Liles W C, Gros P, Kain K C (2008) C5 deficiency and C5a or C5aR blockade protects against cerebral malaria. J Exp Med 205(5): 1133-1143
Pearson & Lipman (1988), Improved tools for biological sequence comparison. Proc. Nat'l.

Acad. Sci. USA 85: 2444
Piccolo M T, Wang Y, Sannomiya P, Piccolo N S, Piccolo M S, Hugli T E, Ward P A, Till G O. 1999. Chemotactic mediator requirements in lung injury following skin burns in rats. Exp Mol Pathol 66(3):220-226.
Pils W and Micura R (2000), Flexible non-nucleotide linkers as loop replacements in short double helical RNAs. Nucleic Acids Res. 28(9):1859-63.
Ricklin D, Lambris J D (2007) Complement-targeted therapeutics. Nat Biotechnol 25(11): 1265-1275
Ricklin D, Lambris J D. 2007 Complement-targeted therapeutics. Nat Biotechnol 25(11): 1265-1275
Riley R D, Sato H, Zhao Z Q, Thourani V H, Jordan J E, Fernandez A X, Ma X L, Hite D R, Rigel D F, Pellas T C, Peppard J, Bill K A, Lappe R W, Vinten-Johansen J. 2000. Recombinant human complement C5a receptor antagonist reduces infarct size after surgical revascularization. J Thorac Cardiovasc Surg 120(2):350-358.
Sim R B, Laich A. 2000. Serine proteases of the complement system. Biochem Soc Trans 28(5):545-550.
Smith & Waterman (1981), Adv. Appl. Math. 2: 482
Sumichika et al (2002) J. Biol. Chem. 277: 49403-49407Thomson J B, Tuschl T, and Eckstein F (1993), Activity of hammerhead ribozymes containing normucleotidic linkers. Nucleic. Acids Res 21: 5600-5603.
Venkatesan N et al. (2003). Curr Med Chem 10:1973
Wagner E, Frank M M (2010) Therapeutic potential of complement modulation. Nat Rev Drug Discov 9(1): 43-56
Walport M J. 2001a. Complement. First of two parts. N Engl J Med 344(14):1058-1066.
Walport M J. 2001b. Complement. Second of two parts. N Engl J Med 344(15):1140-1144.
Wang Y. 2006. Complementary therapies for inflammation. Nat Biotechnol 24(10): 1224-1226
Wincott F, DiRenzo A, Shaffer C, Grimm S, Tracz D, Workman C, Sweedler D, Gonzalez C, Scaringe S, and Usman N (1995). Synthesis, deprotection, analysis and purification of RNA and ribosomes. Nucleic Acids Res. 23: 2677-2684.
Woodruff T M, Arumugam T V, Shiels I A, Reid R C, Fairlie D P, Taylor S M. 2003. A potent human C5a receptor antagonist protects against disease pathology in a rat model of inflammatory bowel disease. J Immunol 171(10):5514-5520.
Woodruff T M, Strachan A J, Dryburgh N, Shiels I A, Reid R C, Fairlie D P, Taylor S M. 2002. Antiarthritic activity of an orally active C5a receptor antagonist against antigen-induced monarticular arthritis in the rat. Arthritis Rheum 46(9):2476-2485.
Yao Y M, Redl H, Bahrami S, Schlag G. 1998. The inflammatory basis of trauma/shock-associated multiple organ failure. Inflamm Res 47(5): 201-210.
Zuiderweg E R, Nettesheim D G, Mollison K W, Carter G W (1989) Tertiary structure of human complement component C5a in solution from nuclear magnetic resonance data. Biochemistry 28(1): 172-185
Zuiderweg E R, Nettesheim D G, Mollison K W, Carter G W. 1989. Tertiary structure of human complement component C5a in solution from nuclear magnetic resonance data. Biochemistry 28(1):172-185.

The features of the present invention disclosed in the specification, the claims, the sequence listing and/or the drawings may both separately and in any combination thereof be material for realizing the invention in various forms thereof.

1. A nucleic acid molecule, capable of binding which binds to C5 or C5a, wherein said nucleic acid comprises an L nucleotide, and said C5 or C5a optionally is glycosylated.

2.-5. (canceled)

6. The nucleic acid molecule according to claim 1, whereby the nucleic acid molecule is selected from the group consisting of type A nucleic acids, type B nucleic acids, type C nucleic acids, type D nucleic acids and type E nucleic acids.

7. The nucleic acid molecule according to claim 6, whereby the type A nucleic acid molecule comprises in 5′→3′ direction a first terminal stretch of nucleotides, a central stretch of nucleotides and a second terminal stretch of nucleotides, whereby
the first terminal stretch of nucleotides and the second terminal stretch of nucleotides optionally hybridize with each other, whereby upon hybridization a double-stranded structure is formed,
the first terminal stretch of nucleotides comprises five to nine nucleotides,
the central stretch of nucleotides comprises a nucleotide sequence of GUCCGAUUGGCGGCACCCUUGCGGGACUGGG
the second terminal stretch of nucleotides comprises five to nine nucleotides.

8.-53. (canceled)

54. The nucleic acid molecule according to claim 6, whereby the type C nucleic acid molecule comprises in 5′→3′ direction a first terminal stretch of nucleotides, a central stretch of nucleotides and a second terminal stretch of nucleotides, whereby
the first terminal stretch of nucleotides and the second terminal stretch of nucleotides optionally hybridize with each other, whereby upon hybridization a double-stranded structure is formed,
the first terminal stretch of nucleotides comprises five to eight nucleotides,
the central stretch of nucleotides comprises a nucleotide sequence of GUGUUUAYUYGCUUAAUAGGGR,
the second terminal stretch of nucleotides comprises five to eight nucleotides.

55.-63. (canceled)

64. The nucleic acid molecule according to claim 6, whereby the type D nucleic acid molecule comprises in 5′→3′ direction a first terminal stretch of nucleotides, a central stretch of nucleotides and a second terminal stretch of nucleotides, whereby
the first terminal stretch of nucleotides and the second terminal stretch of nucleotides optionally hybridize with each other, whereby upon hybridization a double-stranded structure is formed,
the first terminal stretch of nucleotides comprises five to seven nucleotides,
the central stretch of nucleotides comprises a nucleotide sequence of GUUCGGACGUGGCAUGUUCCUUGAYAAACGGUUG,
the second terminal stretch of nucleotides comprises five to seven nucleotides.

65.-70. (canceled)

71. The nucleic acid molecule according to claim 1, whereby the nucleic acid molecule does not inhibit cleavage of C5 to C5a and C5b.

72. (canceled)

73. The nucleic acid molecule according to claim 1, whereby the C5a has an amino acid sequence according to SEQ ID NO:1.

74. The nucleic acid molecule according to preferably claim 1, whereby the C5 comprises an alpha and a beta chain.

75. The nucleic acid molecule according to claim 1, wherein the nucleic acid molecule comprises a modification group.

76. (canceled)

77. The nucleic acid molecule according to claim 75, wherein the modification group is selected from the group consisting of linear poly(ethylene)glycol, branched poly(ethylene)glycol, hydroxyethyl starch, a peptide, a protein, a polysaccharide, a sterol, polyoxypropylene, polyoxyamidate, poly(2-hydroxyethyl)-L-glutamine and polyethylene glycol.

78. The nucleic acid molecule according to claim 75, wherein the modification group is a straight or branched polyethylene glycol.

79. The nucleic acid molecule according to claim 75, wherein the modification group comprises hydroxyethyl starch of molecular weight from about 10,000 to 200,000 Da.

80. The nucleic acid molecule according to claim 75, whereby the modification group is coupled to the nucleic acid molecule via a linker.

81. The nucleic acid molecule according to claim 75, wherein the modification group is coupled to the 5′-terminal nucleotide and/or the 3′-terminal nucleotide of the nucleic acid molecule.

82. (canceled)

83. The nucleic acid molecule according to claim 1, whereby the nucleic acid molecule is an L-nucleic acid molecule.

84.-85. (canceled)

86. A pharmaceutical composition comprising the nucleic acid molecule according to claim 1 and a pharmaceutically acceptable excipient, a pharmaceutically acceptable carrier or a pharmaceutically active agent.

87.-93. (canceled)

94. The nucleic acid molecule according to claim 6, whereby the type E nucleic acid molecule comprises in 5′→3′ direction a first terminal stretch of nucleotides, a central stretch of nucleotides and a second terminal stretch of nucleotides, whereby
the first terminal stretch of nucleotides and the second terminal stretch of nucleotides optionally hybridize with each other, whereby upon hybridization a double-stranded structure is formed,
the first terminal stretch of nucleotides comprises four to five nucleotides,
the central stretch of nucleotides comprises a nucleotide sequence of GACAGGACCAAGGUAAGGGCGGACCGAAAAACCUAG,
the second terminal stretch of nucleotides comprises four to five nucleotides.

95. (canceled)

96. The nucleic acid molecule according to claim 94, whereby the nucleic acid molecule comprises a nucleic acid sequence according to SEQ ID NO:207.

97.-127. (canceled)

128. The nucleic acid of claim 1, comprising one of SEQ ID NOs:73-79.

129. The nucleic acid of claim 74, which binds to said alpha chain having SEQ ID NO:171.


SEQUENCE LISTING (189 sequences)

SEQ ID NO: 1 - protein, 74 aa, Homo sapiens
TLQKKIEEIAAKYKHSVVKKCCYDGACVNNDETCEQRAARISLGPRCIKAFTECCVVASQLRANISHKDMQLGR

SEQ ID NO: 2 - protein, 74 aa, Homo sapiens
  MISC_FEATURE: biotinylated form of C5a
TLQKKIEEIAAKYKHSVVKKCCYDGAAVNNDETCEQRAARISLGPRCIKAFTECCVVASQLRAKISHKDMQLGR

SEQ ID NO: 3 - RNA, 49 nt, Artificial
  synthetic
agcgugcuuguccgauuggcggcacccuugcgggacuggggaguacgcu

SEQ ID NO: 4 - RNA, 45 nt, Artificial
  synthetic
cgugcuuguccgauuggcggcacccuugcgggacuggggaguacg

SEQ ID NO: 5 - RNA, 43 nt, Artificial
  synthetic
gugcuuguccgauuggcggcacccuugcgggacuggggaguac

SEQ ID NO: 6 - RNA, 49 nt, Artificial
  synthetic
agcgugcucguccgauuggcggcacccuugcgggacuggggaguacgcu

SEQ ID NO: 7 - RNA, 46 nt, Artificial
  synthetic
agcgugcuuguccgagcggcacccuugcgggacuggggaguacgcu

SEQ ID NO: 8 - RNA, 47 nt, Artificial
  synthetic
agcgugcuuguccgauuggcggcacccucgggacuggggaguacgcu

SEQ ID NO: 9 - RNA, 43 nt, Artificial
  synthetic
cgugcuuguccgauuggcggcacccucgggacuggggaguacg

SEQ ID NO: 10 - RNA, 41 nt, Artificial
  synthetic
cgugcuuguccgauuggcggcacccgggacuggggaguacg

SEQ ID NO: 11 - RNA, 45 nt, Artificial
  synthetic
cgcgcuuguccgauuggcggcacccuugcgggacuggggagugcg

SEQ ID NO: 12 - RNA, 45 nt, Artificial
  synthetic
cgcgcuuguccgauuggcggcacccuugcgggacuggggagcgcg

SEQ ID NO: 13 - RNA, 43 nt, Artificial
  synthetic
gcgcuuguccgauuggcggcacccuugcgggacuggggagcgc

SEQ ID NO: 14 - RNA, 41 nt, Artificial
  synthetic
gcgcuuguccgauuggcggcacccucgggacuggggagcgc

SEQ ID NO: 15 - RNA, 39 nt, Artificial
  synthetic
gcgcuuguccgauuggcggcacccgggacuggggagcgc

SEQ ID NO: 16 - RNA, 37 nt, Artificial
  synthetic
gcgcuuguccguggcggcacccgggacuggggagcgc

SEQ ID NO: 17 - RNA, 37 nt, Artificial
  synthetic
gcgcuuguccgauucggcacccgggacuggggagcgc

SEQ ID NO: 18 - RNA, 37 nt, Artificial
  synthetic
gcgcuguccgauuggcggcacccgggacugggggcgc

SEQ ID NO: 19 - RNA, 37 nt, Artificial
  synthetic
gcgcuuguccgauuggcggcaccggacuggggagcgc

SEQ ID NO: 20 - RNA, 31 nt, Artificial
  synthetic
guccgauuggcggcacccuugcgggacuggg

SEQ ID NO: 21 - RNA, 48 nt, Artificial
  synthetic
gugcugaacacgccgcguaggacuucaauggaguagaaugggcagcac

SEQ ID NO: 22 - RNA, 48 nt, Artificial
  synthetic
gugcugcaacacgccgaauaggucccgcgcggaagaauggggcagcac

SEQ ID NO: 23 - RNA, 48 nt, Artificial
  synthetic
gugccgccagacgccgaacaggucgcaucgcgaagaaucgggcagcac

SEQ ID NO: 24 - RNA, 48 nt, Artificial
  synthetic
gugcugccagacgccgaacaggucgcaucgcgaagaaucggguagcac

SEQ ID NO: 25 - RNA, 49 nt, Artificial
  synthetic
gugcugcaagacgccgaacagguccaggaagggaagaaucgggcagcac

SEQ ID NO: 26 - RNA, 48 nt, Artificial
  synthetic
gugcugucagacgccgaacaggucgcauugcgaagaaucgggcagcac

SEQ ID NO: 27 - RNA, 47 nt, Artificial
  synthetic
gugcugcuaagacgccggauagguccuuuuaggaagaaucggagcac

SEQ ID NO: 28 - RNA, 47 nt, Artificial
  synthetic
gugcugcaagacgccgaauaggaccgaaguguagaaucgugcagcac

SEQ ID NO: 29 - RNA, 48 nt, Artificial
  synthetic
gugcugagacgccgaacaggaccagcgaaaaugguagaaucgcagcac

SEQ ID NO: 30 - RNA, 16 nt, Artificial
  synthetic
asacgccgvryaggwc

SEQ ID NO: 31 - RNA, 16 nt, Artificial
  synthetic
asacgccgmryaggwc

SEQ ID NO: 32 - RNA, 9 nt, Artificial
  synthetic
gwagaausg

SEQ ID NO: 33 - RNA, 46 nt, Artificial
  synthetic
ggcugaacacgccgcguaggacuucaauggaguagaaugggcagcc

SEQ ID NO: 34 - RNA, 44 nt, Artificial
  synthetic
gcugaacacgccgcguaggacuucaauggaguagaaugggcagc

SEQ ID NO: 35 - RNA, 42 nt, Artificial
  synthetic
cugaacacgccgcguaggacuucaauggaguagaaugggcag

SEQ ID NO: 36 - RNA, 44 nt, Artificial
  synthetic
ggcugaacacgccgcguaggacccaauggguagaaugggcagcc

SEQ ID NO: 37 - RNA, 41 nt, Artificial
  synthetic
ggcugaacacgccgcguaggacccggguagaaugggcagcc

SEQ ID NO: 38 - RNA, 42 nt, Artificial
  synthetic
gcugaacacgccgcguaggacccaauggguagaaugggcagc

SEQ ID NO: 39 - RNA, 42 nt, Artificial
  synthetic
gcggaacacgccgcguaggacccaauggguagaaugggccgc

SEQ ID NO: 40 - RNA, 42 nt, Artificial
  synthetic
gcugcacacgccgcguaggacccaauggguagaaugggcagc

SEQ ID NO: 41 - protein, 74 aa, Bos sp.
MLKKKIEEEAAKYRNAWVKKCCYDGAHRNDDETCEERAARIAIGPECIKAFKSCCAIASQFRADEHHKNMQLGR

SEQ ID NO: 42 - protein, 74 aa, Sus sp.
MLQKKIEEEAAKYKYAMLKKCCYDGAYRNDDETCEERAARIKIGPKCVKAFKDCCYIANQVRAEQSHKNIQLGR

SEQ ID NO: 43 - RNA, 42 nt, Artificial
  synthetic
ggcuaacacgccgcguaggacccaauggguagaaugggagcc

SEQ ID NO: 44 - RNA, 42 nt, Artificial
  synthetic
ggccaacacgccgcguaggacccaauggguagaaugggggcc

SEQ ID NO: 45 - RNA, 42 nt, Artificial
  synthetic
gcccaacacgccgcguaggacccaauggguagaauggggggc

SEQ ID NO: 46 - RNA, 42 nt, Artificial
  synthetic
cgccaacacgccgcguaggacccaauggguagaaugggggcg

SEQ ID NO: 47 - RNA, 42 nt, Artificial
  synthetic
ccggaacacgccgcguaggacccaauggguagaaugggccgg

SEQ ID NO: 48 - RNA, 42 nt, Artificial
  synthetic
cgggaacacgccgcguaggacccaauggguagaaugggcccg

SEQ ID NO: 49 - RNA, 36 nt, Artificial
  synthetic
gcugggcguguuuacuugcuuaauagggggcccagc

SEQ ID NO: 50 - RNA, 36 nt, Artificial
  synthetic
gcugggcguguuuacuugcuuaauaggggucccagc

SEQ ID NO: 51 - RNA, 36 nt, Artificial
  synthetic
gcugggcguguuuacuugcuuaauagggggccuagc

SEQ ID NO: 52 - RNA, 36 nt, Artificial
  synthetic
gcugggcguguuuauuugcuuaauaggggguccagc

SEQ ID NO: 53 - RNA, 36 nt, Artificial
  synthetic
gcugggcguguuuacuugcuuaauagggagcccagc

SEQ ID NO: 54 - RNA, 36 nt, Artificial
  synthetic
gcugggcguguuuacucgcuuaauaggggacccagc

SEQ ID NO: 55 - RNA, 38 nt, Artificial
  synthetic
gcuggggaguguuuacuugcuuaauagggguccccagc

SEQ ID NO: 56 - RNA, 38 nt, Artificial
  synthetic
gcuggggaguguuuacuugcuuaauagggguccucagc

SEQ ID NO: 57 - RNA, 38 nt, Artificial
  synthetic
gcuggggaguguuuacuugcuuaauagggauccuuagc

SEQ ID NO: 58 - RNA, 38 nt, Artificial
  synthetic
gcugaggaguguuuacuugcuuaauagggguccccagc

SEQ ID NO: 59 - RNA, 22 nt, Artificial
  synthetic
guguuuayuygcuuaauagggr

SEQ ID NO: 60 - RNA, 22 nt, Artificial
  synthetic
guguuuacuugcuuaauagggg

SEQ ID NO: 61 - RNA, 34 nt, Artificial
  synthetic
cguggcguguuuacuugcuuaauagggggccacg

SEQ ID NO: 62 - RNA, 34 nt, Artificial
  synthetic
ccgcgcguguuuacuugcuuaauagggggcgcgg

SEQ ID NO: 63 - RNA, 32 nt, Artificial
  synthetic
ugggcguguuuacuugcuuaauagggggccca

SEQ ID NO: 64 - RNA, 32 nt, Artificial
  synthetic
cgggcguguuuacuugcuuaauagggggcccg

SEQ ID NO: 65 - RNA, 32 nt, Artificial
  synthetic
ggggcguguuuacuugcuuaauagggggcccc

SEQ ID NO: 66 - RNA, 32 nt, Artificial
  synthetic
ggggaguguuuacuugcuuaauaggggucccc

SEQ ID NO: 67 - RNA, 30 nt, Artificial
  synthetic
gggcguguuuacuugcuuaauagggggccc

SEQ ID NO: 68 - RNA, 30 nt, Artificial
  synthetic
gggaguguuuacuugcuuaauagggguccc

SEQ ID NO: 69 - RNA, 48 nt, Artificial
  synthetic
guacugcguucggacguggcauguuccuugacaaacgguuggcaguac

SEQ ID NO: 70 - RNA, 48 nt, Artificial
  synthetic
gugcugcguucggacguggcauguuccuugacaaacgguuggcagcac

SEQ ID NO: 71 - RNA, 48 nt, Artificial
  synthetic
gugcuggguucggacguggcauguuccuugauaaacgguugccagcac

SEQ ID NO: 72 - RNA, 34 nt, Artificial
  synthetic
guucggacguggcauguuccuugayaaacgguug

SEQ ID NO: 73 - RNA, 48 nt, Artificial
  synthetic
guguugcguagaauggacauagaggacacgccgcgcaggacgcagcac

SEQ ID NO: 74 - RNA, 48 nt, Artificial
  synthetic
gugcugcgaagaauggacaaaucguacacgccgagcaggucgcaguac

SEQ ID NO: 75 - RNA, 48 nt, Artificial
  synthetic
gugcuggacaggaccaagguaagggcggaccgaaaaaccuagcagcac

SEQ ID NO: 76 - RNA, 49 nt, Artificial
  synthetic
agcgugaacacgccgaauagguccuauaggugggaagaaugggcacgcu

SEQ ID NO: 77 - RNA, 49 nt, Artificial
  synthetic
ccugugcgaagaaugggcccuagggaacacgccgaaaagguugcacagg

SEQ ID NO: 78 - RNA, 48 nt, Artificial
  synthetic
ccugugcgaagcgcucggcgcauaccgaucagguccggcaagcacagg

SEQ ID NO: 79 - RNA, 48 nt, Artificial
  synthetic
cgugcaacacggcgaauagcguccuacaguuaggcagaauggggcacg

SEQ ID NO: 80 - RNA, 49 nt, Artificial; identical to SEQ ID NO: 3
  synthetic

SEQ ID NO: 81 - RNA, 45 nt, Artificial; identical to SEQ ID NO: 4
  synthetic

SEQ ID NO: 82 - RNA, 43 nt, Artificial; identical to SEQ ID NO: 5
  synthetic

SEQ ID NO: 83 - RNA, 49 nt, Artificial; identical to SEQ ID NO: 6
  synthetic

SEQ ID NO: 84 - RNA, 46 nt, Artificial; identical to SEQ ID NO: 7
  synthetic

SEQ ID NO: 85 - RNA, 47 nt, Artificial; identical to SEQ ID NO: 8
  synthetic

SEQ ID NO: 86 - RNA, 43 nt, Artificial; identical to SEQ ID NO: 9
  synthetic

SEQ ID NO: 87 - RNA, 41 nt, Artificial; identical to SEQ ID NO: 10
  synthetic

SEQ ID NO: 88 - RNA, 45 nt, Artificial; identical to SEQ ID NO: 11
  synthetic

SEQ ID NO: 89 - RNA, 45 nt, Artificial; identical to SEQ ID NO: 12
  synthetic

SEQ ID NO: 90 - RNA, 43 nt, Artificial; identical to SEQ ID NO: 13
  synthetic

SEQ ID NO: 91 - RNA, 41 nt, Artificial; identical to SEQ ID NO: 14
  synthetic

SEQ ID NO: 92 - RNA, 39 nt, Artificial; identical to SEQ ID NO: 15
  synthetic

SEQ ID NO: 93 - RNA, 37 nt, Artificial; identical to SEQ ID NO: 16
  synthetic

SEQ ID NO: 94 - RNA, 37 nt, Artificial; identical to SEQ ID NO: 17
  synthetic

SEQ ID NO: 95 - RNA, 37 nt, Artificial; identical to SEQ ID NO: 18
  synthetic

SEQ ID NO: 96 - RNA, 37 nt, Artificial; identical to SEQ ID NO: 19
  synthetic

SEQ ID NO: 97 - RNA, 31 nt, Artificial; identical to SEQ ID NO: 20
  synthetic

SEQ ID NO: 98 - RNA, 48 nt, Artificial; identical to SEQ ID NO: 21
  synthetic

SEQ ID NO: 99 - RNA, 48 nt, Artificial; identical to SEQ ID NO: 22
  synthetic

SEQ ID NO: 100 - RNA, 48 nt, Artificial; identical to SEQ ID NO: 23
  synthetic

SEQ ID NO: 101 - RNA, 48 nt, Artificial; identical to SEQ ID NO: 24
  synthetic

SEQ ID NO: 102 - RNA, 49 nt, Artificial; identical to SEQ ID NO: 25
  synthetic

SEQ ID NO: 103 - RNA, 48 nt, Artificial; identical to SEQ ID NO: 26
  synthetic

SEQ ID NO: 104 - RNA, 47 nt, Artificial; identical to SEQ ID NO: 27
  synthetic

SEQ ID NO: 105 - RNA, 47 nt, Artificial; identical to SEQ ID NO: 28
  synthetic

SEQ ID NO: 106 - RNA, 48 nt, Artificial; identical to SEQ ID NO: 29
  synthetic

SEQ ID NO: 107 - RNA, 16 nt, Artificial
  synthetic
asacgccgvryaggwc

SEQ ID NO: 108 - RNA, 16 nt, Artificial
  synthetic
asacgccgmryaggwc

SEQ ID NO: 109 - RNA, 9 nt, Artificial
  synthetic
gwagaausg

SEQ ID NO: 110 - RNA, 46 nt, Artificial; identical to SEQ ID NO: 33
  synthetic

SEQ ID NO: 111 - RNA, 44 nt, Artificial; identical to SEQ ID NO: 34
  synthetic

SEQ ID NO: 112 - RNA, 42 nt, Artificial; identical to SEQ ID NO: 35
  synthetic

SEQ ID NO: 113 - RNA, 44 nt, Artificial; identical to SEQ ID NO: 36
  synthetic

SEQ ID NO: 114 - RNA, 41 nt, Artificial; identical to SEQ ID NO: 37
  synthetic

SEQ ID NO: 115 - RNA, 42 nt, Artificial; identical to SEQ ID NO: 38
  synthetic

SEQ ID NO: 116 - RNA, 42 nt, Artificial; identical to SEQ ID NO: 39
  synthetic

SEQ ID NO: 117 - RNA, 42 nt, Artificial; identical to SEQ ID NO: 40
  synthetic

SEQ ID NO: 118 - protein, 74 aa, Rattus sp.
LLHQKVEEQAAKYKHRVPKKCCYDGARENKYETCEQRVARVTIGPHCIRAFNECCTIADKIRKESHHKGMLLGR

SEQ ID NO: 119 - protein, 74 aa, Mus sp.
LLRQKIEEQAAKYKHSVPKKCCYDGARVNFYETCEERVARVTIGPLCIRAFNECCTIANKIRKESPHKPVQLGR

SEQ ID NO: 120 - RNA, 42 nt, Artificial; identical to SEQ ID NO: 43
  synthetic

SEQ ID NO: 121 - RNA, 42 nt, Artificial; identical to SEQ ID NO: 44
  synthetic

SEQ ID NO: 122 - RNA, 42 nt, Artificial; identical to SEQ ID NO: 45
  synthetic

SEQ ID NO: 123 - RNA, 42 nt, Artificial; identical to SEQ ID NO: 46
  synthetic

SEQ ID NO: 124 - RNA, 42 nt, Artificial; identical to SEQ ID NO: 47
  synthetic

SEQ ID NO: 125 - RNA, 42 nt, Artificial; identical to SEQ ID NO: 48
  synthetic

SEQ ID NO: 126 - RNA, 36 nt, Artificial; identical to SEQ ID NO: 49
  synthetic

SEQ ID NO: 127 - RNA, 36 nt, Artificial; identical to SEQ ID NO: 50
  synthetic

SEQ ID NO: 128 - RNA, 36 nt, Artificial; identical to SEQ ID NO: 51
  synthetic

SEQ ID NO: 129 - RNA, 36 nt, Artificial; identical to SEQ ID NO: 52
  synthetic

SEQ ID NO: 130 - RNA, 36 nt, Artificial; identical to SEQ ID NO: 53
  synthetic

SEQ ID NO: 131 - RNA, 36 nt, Artificial; identical to SEQ ID NO: 54
  synthetic

SEQ ID NO: 132 - RNA, 38 nt, Artificial; identical to SEQ ID NO: 55
  synthetic

SEQ ID NO: 133 - RNA, 38 nt, Artificial; identical to SEQ ID NO: 56
  synthetic

SEQ ID NO: 134 - RNA, 38 nt, Artificial; identical to SEQ ID NO: 57
  synthetic

SEQ ID NO: 135 - RNA, 38 nt, Artificial; identical to SEQ ID NO: 58
  synthetic

SEQ ID NO: 136 - RNA, 22 nt, Artificial
  synthetic
guguuuayuygcuuaauagggr

SEQ ID NO: 137 - RNA, 22 nt, Artificial
  synthetic
guguuuacuugcuuaauagggg

SEQ ID NO: 138 - RNA, 34 nt, Artificial; identical to SEQ ID NO: 61
  synthetic

SEQ ID NO: 139 - RNA, 34 nt, Artificial; identical to SEQ ID NO: 62
  synthetic

SEQ ID NO: 140 - RNA, 32 nt, Artificial; identical to SEQ ID NO: 63
  synthetic

SEQ ID NO: 141 - RNA, 32 nt, Artificial; identical to SEQ ID NO: 64
  synthetic

SEQ ID NO: 142 - RNA, 32 nt, Artificial; identical to SEQ ID NO: 65
  synthetic

SEQ ID NO: 143 - RNA, 32 nt, Artificial; identical to SEQ ID NO: 66
  synthetic

SEQ ID NO: 144 - RNA, 30 nt, Artificial; identical to SEQ ID NO: 67
  synthetic

SEQ ID NO: 145 - RNA, 30 nt, Artificial; identical to SEQ ID NO: 68
  synthetic

SEQ ID NO: 146 - RNA, 48 nt, Artificial; identical to SEQ ID NO: 69
  synthetic

SEQ ID NO: 147 - RNA, 48 nt, Artificial; identical to SEQ ID NO: 70
  synthetic

SEQ ID NO: 148 - RNA, 48 nt, Artificial; identical to SEQ ID NO: 71
  synthetic

SEQ ID NO: 149 - RNA, 34 nt, Artificial; identical to SEQ ID NO: 72
  synthetic

SEQ ID NO: 150 - RNA, 48 nt, Artificial; identical to SEQ ID NO: 73
  synthetic

SEQ ID NO: 151 - RNA, 48 nt, Artificial; identical to SEQ ID NO: 74
  synthetic

SEQ ID NO: 152 - RNA, 48 nt, Artificial; identical to SEQ ID NO: 75
  synthetic

SEQ ID NO: 153 - RNA, 49 nt, Artificial; identical to SEQ ID NO: 76
  synthetic

SEQ ID NO: 154 - RNA, 49 nt, Artificial; identical to SEQ ID NO: 77
  synthetic

SEQ ID NO: 155 - RNA, 48 nt, Artificial; identical to SEQ ID NO: 78
  synthetic

SEQ ID NO: 156 - RNA, 48 nt, Artificial; identical to SEQ ID NO: 79
  synthetic

SEQ ID NO: 157 - RNA, 51 nt, Artificial
  synthetic
ggagcgugcuuguccgauuggcggcacccuugcgggacuggggaguacgcu

SEQ ID NO: 158 - RNA, 43 nt, Artificial
  synthetic
gggcgcuuguccgauuggcggcacccucgggacuggggagcgc

SEQ ID NO: 159 - RNA, 46 nt, Artificial
  synthetic
ggggcugaacacgccgcguaggacccaauggguagaaugggcagcc

SEQ ID NO: 160 - RNA, 44 nt, Artificial
  synthetic
gggcugaacacgccgcguaggacccaauggguagaaugggcagc

SEQ ID NO: 161 - RNA, 38 nt, Artificial
  synthetic
gggcugggcguguuuacuugcuuaauagggggcccagc

SEQ ID NO: 162 - RNA, 34 nt, Artificial
  synthetic
ggugggcguguuuacuugcuuaauagggggccca

SEQ ID NO: 163 - RNA, 34 nt, Artificial
  synthetic
ggggggcguguuuacuugcuuaauagggggcccc

SEQ ID NO: 164 - RNA, 32 nt, Artificial
  synthetic
gggggcguguuuacuugcuuaauagggggccc

SEQ ID NO: 165 - RNA, 50 nt, Artificial
  synthetic
ggguacugcguucggacguggcauguuccuugacaaacgguuggcaguac

SEQ ID NO: 166 - RNA, 50 nt, Artificial
  synthetic
gggugcugcguucggacguggcauguuccuugacaaacgguuggcagcac

SEQ ID NO: 167 - RNA, 41 nt, Artificial; identical to SEQ ID NO: 14
  synthetic

SEQ ID NO: 168 - RNA, 44 nt, Artificial; identical to SEQ ID NO: 36
  synthetic

SEQ ID NO: 169 - RNA, 36 nt, Artificial; identical to SEQ ID NO: 49
  synthetic

SEQ ID NO: 170 - RNA, 32 nt, Artificial; identical to SEQ ID NO: 65
  synthetic

SEQ ID NO: 171 - protein, 999 aa, Homo sapiens
TLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGIGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 172 - protein, 655 aa, Homo sapiens
QEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVILNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEIL

SEQ ID NO: 173 - RNA, 9 nt, Artificial
  synthetic
nnngygcny

SEQ ID NO: 174 - RNA, 9 nt, Artificial
  synthetic
gngyrcnnn

SEQ ID NO: 175 - RNA, 7 nt, Artificial
  synthetic
ngygcnu

SEQ ID NO: 176 - RNA, 7 nt, Artificial
  synthetic
gngygcn

SEQ ID NO: 177 - RNA, 8 nt, Artificial
  synthetic
nnsbbnnn

SEQ ID NO: 178 - RNA, 8 nt, Artificial
  synthetic
nnnvvsnn

SEQ ID NO: 179 - RNA, 8 nt, Artificial
  synthetic
nngcynnn

SEQ ID NO: 180 - RNA, 8 nt, Artificial
  synthetic
nnnagcnn

SEQ ID NO: 181 - RNA, 8 nt, Artificial
  synthetic
nngccnnn

SEQ ID NO: 182 - RNA, 8 nt, Artificial
  synthetic
nnnkvgnm

SEQ ID NO: 183 - RNA, 8 nt, Artificial
  synthetic
dnybhnnn

SEQ ID NO: 184 - DNA, 22 bp, Artificial
  synthetic
atgctacaagagaagatagaag

SEQ ID NO: 185 - DNA, 25 bp, Artificial
  synthetic
ctagcatgcttaccttcccaattgc

SEQ ID NO: 186 - protein, 74 aa, Macaca mulatta
MLQEKIEEIAAKYKHLVVKKCCYDGVRINHDETCEQRAARISVGPRCVKAFTECCVVASQLRANNSHKDLQLGR

SEQ ID NO: 187 - RNA, 32 nt, Artificial
  synthetic
ccccgggggauaauucguucauuugugcgggg

SEQ ID NO: 188 - RNA, 8 nt, Artificial
  synthetic
nnssbnnn

SEQ ID NO: 189 - RNA, 8 nt, Artificial
  synthetic
nnnvssnn